(19) United States
(12) Patent Application Publication
(10) Pub. No.: US 2024/0082274 A1
(43) Pub. Date: Mar. 14, 2024

(54) FUNGAL COMPOUND COMPOSITIONS AND METHODS FOR MODULATING INFLAMMATION

(71) Applicant: TURTLE BEAR HOLDINGS, LLC, Shelton, WA (US)

(72) Inventor: Paul E. STAMETS, Shelton, WA (US)

(21) Appl. No.: 18/498,563

(22) Filed: Oct. 31, 2023

Related U.S. Application Data
(63) Continuation of application No. 17/738,925, filed on May 6, 2022.
(60) Provisional application No. 63/185,625, filed on May 7, 2021.

(51) Int. Cl. A61K 31/675; A61K 36/06; A61K 45/06; A61P 25/28; A61P 29/00

(52) U.S. Cl. CPC A61K 31/675; A61K 36/06; A61K 45/06; A61P 25/28; A61P 29/00

(57) ABSTRACT

Described herein are fungal compound compositions and methods for treating, prophylaxis of, or ameliorating symptoms of one or more adverse reactions triggered by an infectious disease or condition that increases an anti-inflammatory response in a subject with such compositions. In one aspect, the composition comprises one or more tryptamines or in pure form or extracts from psilocybin containing mushrooms, or combinations thereof, optionally combined with one or more erinacines or hericenones in pure form, extracts from Hericium mushroom species (e.g., H. erinaceus, H. coralloides, H. ramosum), or combinations thereof, optionally one or more adversive compounds, optionally one or more monoamine oxidase inhibitor (MAOI) compounds, and optionally one or more pharmaceutically acceptable excipients.

### CROSS-REFERENCE TO RELATED APPLICATIONS

This application is a continuation of U.S. Non-Provisional patent application Ser. No. 17/738,925, filed on May 6, 2022, which claims priority to U.S. Provisional Patent Application No. 63/185,625, filed on May 7, 2021, the entire contents of each of which are fully incorporated herein by reference.

### TECHNICAL FIELD

Described herein are fungal compound compositions and methods for treating, prophylaxis of, or ameliorating symptoms of one or more adverse reactions triggered by an infectious disease or condition that increases an anti-inflammatory response in a subject with such compositions. In one aspect, the composition comprises one or more tryptamines or in pure form or extracts from psilocybin containing mushrooms, or combinations thereof, optionally combined with one or more erinacines or hericenones in pure form, extracts from Hericium mushroom species (e.g., H. erinaceus, H. coralloides, H. ramosum), or combinations thereof, optionally one or more adversive compounds, optionally one or more monoamine oxidase inhibitor (MAOI) compounds, and optionally one or more pharmaceutically acceptable excipients.

### BACKGROUND

Mushrooms have been embraced for centuries due to their nutritional and medicinal properties. They have been historically used in the treatment of infectious disease, gastrointestinal disorders, and asthmatic conditions, as well as to support overall wellbeing. Fungi now occupy their own kingdom, but they were once considered plants due to their resemblance and root-like structures. One of many characteristics that separate fungal from plant organisms is the cell wall structure. The cell walls of fungi contain chitin, a modified form of the polysaccharide cellulose. Chitin is comprised of β-(1→4)-linked N-acetylglucosamine monomers, whereas cellulose is comprised of β-(1→4)-linked glucose units. Chitin degrades into a mixture of shorter-chained polysaccharides along with monosaccharide products. This degradation can occur with a variety of processing techniques that implement heat and drying.

Contemporary research has mainly focused on the broad immune activity of mushrooms. Mushroom polysaccharides possess documented immunomodulatory properties, specifically through the activation of natural killer cells, macrophages, and neutrophils, as well as induction of innate immune cytokines and interleukins. β-glucans, proteoglycans, and heteroglucans are classes of polymers present in the cell walls of fungi. The generic term β-glucan refers to the polymeric form of glucose residues connected by β-(1→3), β-(1→4), and β-(1→6)-linkages. The type of β-glucans isolated from fungi consist mainly of a linear backbone of β-(1→3) glucose monomers and side branches comprised of β-(1→3) and β-(1→6)-linked oligosaccharides.

The most widely studied β-glucans are comprised of (1→3)-β, and (1,6)-β linkages, which exhibit immunostimulatory and antitumor properties. These polysaccharides are ligands for the dectin-1 and toll-like receptor 2 (TLR-2) receptor systems expressed on macrophages and dendritic cells, inducing NK cells, neutrophils, T-cells, B-cells, as well as TNF-α, IL-4, and IL-6 signaling. The Complement Receptor-3 (CD11b/CD18) in context of extracellular matrix is also involved in immune responses to fungal β-glucans. While the immune activating, pro-inflammatory properties of fungal water-insoluble β-glucans are well documented, the immune modulating effects of fungal non-β-glucan-fractions are less recognized.

While prior research on medicinal mushrooms has primarily focused on the solid, β-glucan-rich fraction, and β-glucan mediated responses are clearly important, focusing on this compound class in isolation clearly does not reflect the overall bioactivity of a complex blend when consumed for immune support. Emerging evidence suggests that a blend of mushrooms may provide additive or synergistic effects on the host immune response.

### SUMMARY

One embodiment described herein is a composition comprising: one or more tryptamines, salts thereof, or combinations thereof; and extracts or isolates from Hericium erinaceus mushroom species, erinacines, hericenones, or combinations thereof. In one aspect, the one or more tryptamines may be psilocybin, psilocin, norpsilocin, baeocystin, norbaeocystin, N,N-dimethyltryptamine (DMT), or combinations thereof. In another aspect, the composition may comprise about 1 ng to about 10 mg, about 10 mg to about 100 mg, about 10 mg to about 20 mg, about 20 mg to about 50 mg, about 20 mg to about 100 mg, about 1 ng to about 20 mg, about 1 ng to about 50 mg, or about 1 ng to about 100 mg of the one or more tryptamines, salts thereof, or combinations thereof. In another aspect, the composition may comprise about 1 ng to about 2000 mg of the extracts or isolates from Hericium erinaceus mushroom species, erinacines, hericenones, or combinations thereof. In another aspect, the composition may further comprise a monoamine oxidase inhibitor. In another aspect, the composition may comprise about 70 mg to about 200 mg of the monoamine oxidase inhibitor. In another aspect, the monoamine oxidase inhibitor may be Norharman, Harmine, 1,2,3,4-tetrahydro-β-carboline-3-carboxylic acid, 1-methyl-1,2,3,4-tetrahydro-β-carboline-3-carboxylic acid, 1-methyl-2,3,4,9-tetrahydro-1H-β-carboline-1,3-dicarboxylic acid, Harmaline, N-methoxy-1-vinyl-β-carboline, ethyl 9H-β-arboline-3-carboxylate, 1-fury)-β-carboline-3-carboxylic acid, 1-[5-(methoxymethyl)-2-furyl]-9H-β-carboline-3-carboxylic acid, 6-hydroxy-3-(6-hydroxy-1H-indol-3-yl)-9H-β-carboline-4-carboxylic acid, Strictosidine, (1S)-1-{[(2S,3R,4S)-2-(β-L-glucopyranosyloxy)-5-(methoxycarbonyl)-3-vinyl-3,4-dihydro-2H-pyran-4-yl]methyl}-2,3,4,9-tetrahydro-1H-β-carboline-1,3-dicarboxylic acid, or combinations thereof. Another embodiment described herein is a composition comprising: psilocybin, psilocin, norpsilocin, baeocystin, norbaeocystin, N,N-dimethyltryptamine (DMT), salts thereof, or combinations thereof; and an erinacine or hericenone in pure form, extracts or isolates from Hericium erinaceus mushroom species, or combinations thereof. In one aspect, the composition may comprise about 1 ng to about 10 mg, about 10 mg to about 100 mg, about 10 mg to about 20 mg, about 20 mg to about 50 mg, about 20 mg to about 100 mg, about 1 ng to about 20 mg, about 1 ng to about 50 mg, or about 1 ng to about 100 mg of the psilocybin, psilocin, norpsilocin, baeocystin, norbaeocystin, N,N-dimethyltryptamine (DMT), salts thereof, or combinations thereof. In another aspect, the composition may comprise about 1 ng to about 2000 mg of the erinacine or hericenone in pure form, extracts or isolates from Hericium erinaceus mushroom species, or combinations thereof. In another aspect, the composition may further comprise a monoamine oxidase inhibitor. In another aspect, the composition may comprise about 70 mg to about 200 mg of the monoamine oxidase inhibitor. In another aspect, the monoamine oxidase inhibitor may be Norharman, Harmine, 1,2,3,4-tetrahydro-β-carboline-3-carboxylic acid, 1-methyl-1,2,3,4tetrahydro-β-carboline-3-carboxylic acid, 1-methyl-2,3,4,9-tetrahydro-1H-β-carboline-1,3-dicarboxylic acid, Harmaline, N-methoxy-1-vinyl-β-carboline, ethyl 9H-β-arboline-3-carboxylate, 1-fury)-β-carboline-3-carboxylic acid, 1-[5-(methoxymethyl)-2-furyl]-9H-β-carboline-3-carboxylic acid, 6-hydroxy-3-(6-hydroxy-1H-indol-3-yl)-9H-β-carboline-4-carboxylic acid, Strictosidine, (1S)-1-{[(2S,3R,4S)-2-(β-L-glucopyranosyloxy)-5-(methoxycarbonyl)-3-vinyl-3,4-dihydro-2H-pyran-4-yl]methyl}-2,3,4,9-tetrahydro-1H-β-carboline-1,3-dicarboxylic acid, or combinations thereof.

Another embodiment described herein is a method for treating or modulating an inflammatory response triggered by an infectious disease or condition, the method comprising: administering a composition to a subject in need thereof, the composition comprising: one or more tryptamines, salts thereof, or combinations thereof.

Another embodiment described herein is a method for treating or modulating an inflammatory response triggered by an infectious disease or condition, the method comprising: administering a composition to a subject in need thereof, the composition comprising: one or more tryptamines, salts thereof, or combinations thereof; and extracts or isolates from Hericium erinaceus mushroom species, erinacines, hericenones, or combinations thereof. In one aspect, the composition may comprise about 1 ng to about 10 mg, about 10 mg to about 100 mg, about 10 mg to about 20 mg, about 20 mg to about 50 mg, about 20 mg to about 100 mg, about 1 ng to about 20 mg, about 1 ng to about 50 mg, or about 1 ng to about 100 mg of the one or more tryptamines, salts thereof, or combinations thereof. In another aspect, the one or more tryptamines may be psilocybin, psilocin, norpsilocin, baeocystin, norbaeocystin, N,N-dimethyltryptamine (DMT), or combinations thereof. In another aspect, the composition may comprise about 1 ng to about 2000 mg of the extracts or isolates from Hericium erinaceus mushroom species, erinacines, hericenones, or combinations thereof. In another aspect, the composition may further comprise a monoamine oxidase inhibitor. In another aspect, the composition may comprise about 70 mg to about 200 mg of the monoamine oxidase inhibitor. In another aspect, the monoamine oxidase inhibitor may be Norharman, Harmine, 1,2,3,4-tetrahydro-β-carboline-3-carboxylic acid, 1-methyl-1,2,3,4-tetrahydro-β-carboline-3-carboxylic acid, 1-methyl-2,3,4,9-tetrahydro-1H-β-carboline-1,3-dicarboxylic acid, Harmaline, N-methoxy-1-vinyl-β-carboline, ethyl 9H-β-arboline-3-carboxylate, 1-furyl-β-carboline-3-carboxylic acid, 1-[5-(methoxymethyl)-2-furyl]-9H-β-carboline-3-carboxylic acid, 6-hydroxy-3-(6-hydroxy-1H-indol-3-yl)-9H-β-carboline-4-carboxylic acid, Strictosidine, (1S)-1-{[(2S,3R,4S)-2-(β-L-glucopyranosyloxy)-5-(methoxycarbonyl)-3-vinyl-3,4-dihydro-2H-pyran-4-yl]methyl}-2,3,4,9-tetrahydro-1H-β-carboline-1,3-dicarboxylic acid, or combinations thereof. In another aspect, the inflammatory response may be cytokine storm. In another aspect, the infectious disease or condition may be a viral infection, a bacterial infection, or a parasitic infection. In another aspect, the viral infection may be Paramyxoviridae (respiratory syncytial virus (RSV), parainfluenza virus (PIV), metapneumovirus (MPV), enteroviruses), Picornaviridae (Rhinovirus, RV), Coronaviridae (CoV), Adenoviridae (Adenovirus), Parvoviridae (HBoV), Orthomyxoviridae (influenza A, B, C, D, Isavirus, Thogotovirus, Quaranjavirus), Herpesviridae (human herpes viruses, Varicella zoster virus, Epstein-Barr virus, cytomegalovirus), avian influenza, smallpox, pandemic influenza, or adult respiratory distress syndrome (ARDS). In another aspect, the bacterial infection may be Streptococcus pneumoniae, Mycobacterium tuberculosis, Bordetella pertussis, Haemophilus influenzae, Moraxella catarrhalis, Pseudomonas aeruginosa, Stenotrophomonas maltophila, Staphylococcus aureus, Streptococcus pyogenes, Neisseria meningitidis, Klebsiella pneumoniae, or Non-tuberculosis Mycobacterium. In another aspect, the parasitic infection may be malaria. In another aspect, inflammation may be reduced and neuroregeneration is induced in the subject. In another aspect, neuroregeneration may comprise neurite outgrowth. In another aspect, the infectious disease or condition may cause neurological damage in the subject and the method may result in treatment of the neurological damage.

Another embodiment disclosed herein is a method for inducing expression of an anti-inflammatory cytokine, the method comprising administering a composition to a subject in need thereof, the composition comprising: one or more tryptamines, salts thereof, or combinations thereof.

Another embodiment disclosed herein is a method for inducing expression of an anti-inflammatory cytokine, the method comprising administering a composition to a subject in need thereof, the composition comprising: one or more tryptamines, salts thereof, or combinations thereof; and extracts or isolates from Hericium erinaceus mushroom species, erinacines, hericenones, or combinations thereof. In one aspect, the composition may comprise about 1 ng to about 10 mg, about 10 mg to about 100 mg, about 10 mg to about 20 mg, about 20 mg to about 50 mg, about 20 mg to about 100 mg, about 1 ng to about 20 mg, about 1 ng to about 50 mg, or about 1 ng to about 100 mg of the one or more tryptamines, salts thereof, or combinations thereof. In another aspect, the one or more tryptamines may be psilocybin, psilocin, norpsilocin, baeocystin, norbaeocystin, N,N-dimethyltryptamine (DMT), or combinations thereof. In another aspect, the composition may comprise about 1 ng to about 2000 mg of the extracts or isolates from Hericium erinaceus mushroom species, erinacines, hericenones, or combinations thereof. In another aspect, the composition may further comprise a monoamine oxidase inhibitor. In another aspect, the composition may comprise about 70 mg to about 200 mg of the monoamine oxidase inhibitor. In another aspect, the monoamine oxidase inhibitor may be Norharman, Harmine, 1,2,3,4-tetrahydro-β-carboline-β-carboxylic acid, 1-methyl-1,2,3,4-tetrahydro-β-carboline-3-carboxylic acid, 1-methyl-2,3,4,9-tetrahydro-1H-β-carboline-1,3-dicarboxylic acid, Harmaline, N-methoxy-1-vinyl-β-carboline, ethyl 9H-β-arboline-3-carboxylate, 1-furyl-β-carboline-3-carboxylic acid, 1-[5-(methoxymethyl)-2-furyl]-9H-β-carboline-3-carboxylic acid, 6-hydroxy-3-(6-hydroxy-1H-indol-3-yl)-9H-β-carboline-4-carboxylic acid, Strictosidine, (1S)-1-{[(2S,3R,4S)-2-(β-L-glucopyranosyloxy)-5-(methoxycarbonyl)-3-vinyl-3,4-dihydro-2H-pyran-4-yl]methyl}-2,3,4,9-tetrahydro-1H-β-carboline-1,3-dicarboxylic acid, or combinations thereof. In another aspect, the anti-inflammatory cytokine may be IL-4, IL-10, IL-1RA, or a combination thereof. In another aspect, inflammation may be reduced and neuroregeneration is induced in the subject. In another aspect, neuroregeneration may comprise neurite outgrowth.

Another embodiment described herein is a method for treating or modulating an inflammatory response triggered by an infectious disease or condition by inducing expression of one or more anti-inflammatory cytokines selected from the group of IL-4, IL-10, and IL-1RA, the method comprising: administering a composition to a subject in need thereof, the composition comprising: about 1 ng to about 10 mg, about 10 mg to about 100 mg, about 10 mg to about 20 mg, about 20 mg to about 50 mg, about 20 mg to about 100 mg, about 1 ng to about 20 mg, about 1 ng to about 50 mg, or about 1 ng to about 100 mg of one or more tryptamines, salts thereof, or combinations thereof; and about 10 ng to about 2000 mg of extracts or isolates from Hericium erinaceus mushroom species, erinacines, hericenones, or combinations thereof.

Another embodiment described herein is a method for treating or modulating an inflammatory response triggered by an infectious disease or condition by inducing expression of one or more anti-inflammatory cytokines selected from the group of IL-4, IL-10, and IL-1RA, the method comprising: administering a composition to a subject in need thereof, the composition comprising: about 1 ng to about 10 mg, about 10 mg to about 100 mg, about 10 mg to about 20 mg, about 20 mg to about 50 mg, about 20 mg to about 100 mg, about 1 ng to about 20 mg, about 1 ng to about 50 mg, or about 1 ng to about 100 mg of one or more tryptamines, salts thereof, or combinations thereof; about 1 ng to about 2000 mg of extracts or isolates from Hericium erinaceus mushroom species, erinacines, hericenones, or combinations thereof; and about 70 mg to about 200 mg of a monoamine oxidase inhibitor. In one aspect, the inflammatory response may be cytokine storm. In another aspect, the infectious disease or condition may be a viral infection, a bacterial infection, or a parasitic infection. In another aspect, the viral infection may be Paramyxoviridae (respiratory syncytial virus (RSV), parainfluenza virus (PIV), metapneumovirus (MPV), enteroviruses), Picornaviridae (Rhinovirus, RV), Coronaviridae (CoV), Adenoviridae (Adenovirus), Parvoviridae (HBoV), Orthomyxoviridae (influenza A, B, C, D, Isavirus, Thogotovirus, Quaranjavirus), Herpesviridae (human herpes viruses, Varicella zoster virus, Epstein-Barr virus, cytomegalovirus), avian influenza, smallpox, pandemic influenza, or adult respiratory distress syndrome (ARDS). In another aspect, the bacterial infection may be Streptococcus pneumoniae, Mycobacterium tuberculosis, Bordetella pertussis, Haemophilus influenzae, Moraxella catarrhalis, Pseudomonas aeruginosa, Stenotrophomonas maltophila, Staphylococcus aureus, Streptococcus pyogenes, Neisseria meningitidis, Klebsiella pneumoniae, or Non-tuberculosis Mycobacterium. In another aspect, the parasitic infection may be malaria. In another aspect, the one or more tryptamines may be psilocybin, psilocin, norpsilocin, baeocystin, norbaeocystin, N,N-dimethyltryptamine (DMT), or combinations thereof. In another aspect, the monoamine oxidase inhibitor may be Norharman, Harmine, 1,2,3,4-tetrahydro-β-carboline-3-carboxylic acid, 1-methyl-1,2,3,4-tetrahydro-β-carboline-3-carboxylic acid, 1-methyl-2,3,4,9-tetrahydro-1H-β-carboline-1,3-dicarboxylic acid, Harmaline, N-methoxy-1-vinyl-β-carboline, ethyl 9H-β-arboline-3-carboxylate, 1-furyl-β-carboline-3-carboxylic acid, 1-[5-(methoxymethyl)-2-furyl]-9H-β-carboline-3-carboxylic acid, 6-hydroxy-3-(6-hydroxy-1H-indol-3-yl)-9H-β-carboline-4-carboxylic acid, Strictosidine, (1S)-1-{[(2S,3R,4S)-2-(β-L-glucopyranosyloxy)-5-(methoxycarbonyl)-3-vinyl-3,4-dihydro-2H-pyran-4-yl]methyl}-2,3,4,9-tetrahydro-1H-β-carboline-1,3-dicarboxylic acid, or combinations thereof. In another aspect, inflammation may be reduced and neuroregeneration is induced in the subject. In another aspect, neuroregeneration may comprise neurite outgrowth. In another aspect, the infectious disease or condition may cause neurological damage in the subject and the method may result in treatment of the neurological damage.

### BRIEF DESCRIPTION OF THE DRAWINGS

FIG. 1A-B show the impact of different treatments on the induction of the anti-inflammatory cytokine IL-10 in 1321N1 human brain cells and human peripheral blood mononuclear cells (PBMCs). FIG. 1A shows the effect of treatment with He EtOH, Baeocystin, Norbaeocystin, Norpsilocin, He-Baeocystin, He-Norbaeocystin, He-Norpsilocin, and vehicle control on IL-10 expression (pg/mL) in 1321N1 human brain cells. FIG. 1B shows the effect of treatment with Norbaeocystin, Norpsilocin, and vehicle control on IL-10 expression (pg/mL) in PBMCs under standard non-inflammatory conditions (N=4 for all treatments and vehicle control; *p≤0.05, **p≤0.01, ***p≤0.001, ****p≤0.0001).

FIG. 2A-D show the synergistic effects of different combination formulations on JAK1 binding affinity, as compared to the calculated theoretical sum of each individual component. FIG. 2A shows a 2.6× synergistic effect for a He-Norpsilocin-Niacin formulation (250 μg/mL) on JAK1 binding affinity. FIG. 2B shows a 2.4× synergistic effect for a PEP-Psilocin formulation (Human dose equivalency: 2.2 mg PEP, 0.12 mg Psilocin) on JAK1 binding affinity. PEP represents a putative erinacine peak, a column derived He fraction enriched with erinacines. FIG. 2C shows a 2× synergistic effect for a DMT-Niacin formulation (Human dose equivalency: 1.1 mg DMT, 1.1 mg Niacin) on JAK1 binding affinity. FIG. 2D shows a 1.9× synergistic effect for a DMT-Niacin formulation (Human dose equivalency: 10 mg DMT, 10 mg Niacin) on JAK1 binding affinity.

FIG. 3A-C show the synergistic effects of different combination formulations on JNK3 binding affinity, as compared to the calculated theoretical sum of each individual component. FIG. 3A shows an 8× synergistic effect for a Psilocin-Niacin formulation (Human dose equivalency: 0.04 mg Psilocin, 0.04 mg Niacin) on JNK3 binding affinity. FIG. 3B shows a 2.5× synergistic effect for an Erinacine C-Psilocin formulation (Human dose equivalency: 0.24 mg Erinacine C, 0.01 mg Psilocin) on JNK3 binding affinity. FIG. 3C shows a 2× synergistic effect for an Erinacine C-Psilocin-Niacin formulation (Human dose equivalency: 58.3 mg Erinacine C, 3.3 mg Psilocin, 3.3 mg Niacin) on JNK3 binding affinity.

FIG. 4A-F show the synergistic effects of different combination formulations on TRKA binding affinity, as compared to the calculated theoretical sum of each individual component. FIG. 4A shows a 1.3× synergistic effect for a He-baeocystin-norbaeocystin formulation on TRKA binding affinity. FIG. 4B shows a maximum calculable value (MCV) synergistic effect for a He-DMT-Niacin formulation (Human dose equivalency: 194 mg He, 1.1 mg DMT, 1.1 mg Niacin) on TRKA binding affinity. FIG. 4C shows a MCV synergistic effect for an Erinacine C-Niacin formulation (Human dose equivalency: 0.08 mg Erinacine C, 0.005 mg Niacin) on TRKA binding affinity. FIG. 4D shows a MCV synergistic effect for an Erinacine C-Psilocin formulation (Human dose equivalency: 0.08 mg Erinacine C, 0.005 mg Psilocin) on TRKA binding affinity. FIG. 4E shows a MCV synergistic effect for an Erinacine C-Psilocin-Niacin formulation (Human dose equivalency: 0.72 mg Erinacine C, 0.04 mg Psilocin, 0.04 mg Niacin) on TRKA binding affinity. FIG. 4F shows a MCV synergistic effect for a DMT-Niacin formulation (Human dose equivalency: 3.3 mg DMT, 3.3 mg Niacin) on TRKA binding affinity.

FIG. 5A-C show the effects of He, PEP, psilocin, niacin, and He-psilocin-niacin and PEP-psilocin-niacin combination formulations on neurite outgrowth in PC12 cells. These data demonstrate that the combination of He, psilocin, and niacin provides the most robust induction of neurite extension out of the different treatments tested. FIG. 5A shows representative images for each of the tested treatments, with arrows indicating specific regions of neurite outgrowth. Cells were incubated for 6 days following treatment before imaging was conducted. FIG. 5B shows a graph of the mean neurite length (pixels) for each treatment. FIG. 5C shows the mean neurite growth for each treatment as a relative % of vehicle control. N=4 for He myc EtOAc and He-psilocin-niacin combination treatments; N=3 for PEP and PEP-psilocin-niacin combination treatments; *p<0.05, **p<0.01.

FIG. 6 shows the impact of different He myc SOP EtOAc treatment concentrations on IL-6 expression (pg/mL) in human PBMCs under LPS-induced (100 ng/mL) inflammatory conditions (N=3 for all He myc SOP EtOAc treatments, N=4 for vehicle control; *p≤0.05, **p≤0.01).

FIG. 7 shows the impact of different Niacin treatment concentrations on IL-6 expression (pg/mL) in human PBMCs under LPS-induced (100 ng/mL) inflammatory conditions (N=3 for all Niacin treatments, N=4 for vehicle control; *p≤0.05).

FIG. 8 shows the impact of different PEP treatment concentrations on IL-6 expression (pg/mL) in human PBMCs under LPS-induced (100 ng/mL) inflammatory conditions (N=3 for all PEP treatments, N=4 for vehicle control; *p≤0.05, **p≤0.01).

FIG. 9 shows the impact of different Psilocin treatment concentrations on IL-6 expression (pg/mL) in human PBMCs under LPS-induced (100 ng/mL) inflammatory conditions (N=3 for all Psilocin treatments, N=4 for vehicle control; *p≤0.05).

FIG. 10 shows the impact of different He-Psilocin-Niacin formulation treatment concentrations on IL-6 expression (pg/mL) in human PBMCs under LPS-induced (100 ng/mL) inflammatory conditions (N=3 for all He combination treatments, N=4 for vehicle control; *p≤0.05).

FIG. 11 shows the impact of different PEP-Psilocin-Niacin formulation treatment concentrations on IL-6 expression (pg/mL) in human PBMCs under LPS-induced (100 ng/mL) inflammatory conditions (N=3 for all PEP combination treatments, N=4 for vehicle control; *p≤0.05)

FIG. 12 shows the impact of different Norbaeocystin treatment concentrations on IL-6 expression (Absorbance at 450 nm) in human PBMCs under LPS-induced (100 ng/mL) inflammatory conditions (N=4 for all Norbaeocystin treatments and vehicle control; *p≤0.05).

FIG. 13 shows the impact of different treatments on the expression of the pro-inflammatory protein TNF-α (Absorbance at 450 nm) in human PBMCs under LPS-induced (100 ng/mL) inflammatory conditions (N=3 for all treatments, N=4 for vehicle control; *p≤0.05, **p≤0.01).

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, all percentages (%) used for compositions or formulations refer to mass (or weight, w/w) percent unless noted otherwise.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “therapeutic composition” and “pharmaceutical composition” can be used interchangeably and refer to a combination of at least two ingredients.

As used herein, “formulation,” “composition,” “therapeutic composition,” and “pharmaceutical composition” can be used interchangeably and refer to a combination of at least two ingredients. In some embodiments, at least one ingredient may be an active agent or otherwise have properties that exert physiologic activity when administered to a subject. For example, a mixture including at least two ingredients (e.g., water and norpsilocin) and is itself a composition or formulation.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations.

The term “dosage” as used herein refers to the administering of a specific amount, number, and frequency of doses over a specified period of time, typically 1 day.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, the terms “effective amount,” “therapeutically effective amount,” or “therapeutically effective rate(s)” refers to a substantially non-toxic, but sufficient amount or delivery rates of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. It is understood that various biological factors may affect the ability of an agent to perform its intended task. Therefore, an “effective amount,” “therapeutically effective amount,” or “therapeutically effective rate(s)” may be dependent in some instances on such biological factors. For example, the precise determination of what would be considered an effective dose may be based on factors individual to each patient, including, but not limited to, the patient's age, size, type or extent of disease, stage of the disease, route of administration, the type or extent of supplemental therapy used, ongoing disease process and type of treatment desired (e.g., aggressive vs. conventional treatment). Further, while the achievement of therapeutic effects may be measured by a physician or a qualified medical practitioner using evaluations known in the art, it is recognized that individual variation and response to treatments may make the achievement of therapeutic effects a subjective decision. An appropriate “effective” amount in any individual case may be determined using techniques, such as a dose escalation study. The dose could be administered in one or more administrations. The determination of a therapeutically effective amount or delivery rate is well within the ordinary skill in the art of pharmaceutical sciences and medicine.

As used herein, the terms “subject,” “study participant,” “participant,” and “patient” interchangeably refer to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamsters, guinea pig, cat, dog, rat, and mouse. The mammal can be a primate such as a human. The mammal can be a non-human primate such as, for example, monkey, cynomolgus monkey, rhesus monkey, chimpanzee, gorilla, orangutan, and gibbon. The subject may be of any age or stage of development, such as, for example, an adult, an adolescent, or an infant. The subject may be male. The subject may be female. In some embodiments, the subject has a specific genetic marker. The subject may be undergoing other forms of treatment.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, the terms “sample” or “test sample” refers any sample in which the presence and/or level of a target is to be detected or determined or any sample treated with the compositions as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

As used herein, the terms “erinacines” and “hericenones” refer to the cyathin diterpenoids erinacine, hericenone, and related compounds. The compounds may be synthetic or natural products isolated from or extracted from H. erinaceus, H. coralloides, H. ramosum. Exemplary compounds include Erinacine A, Erinacine B, Erinacine C, Erinacine D, Erinacine E, Erinacine F, Erinacine G, Erinacine H, Erinacine I, Erinacine J, Erinacine K, Erinacine P, Erinacine Q, Erinacine R, Erinacol, other Erinacines Hericenone A, Hericenone B, Hericenone C, Hericenone D, Hericenone E, Hericenone F, Hericenone G, Hericenone H, other hericenones, or pharmaceutically acceptable salts, hydrates, solvates, prodrugs, stereoisomers, or tautomers thereof. In contrast to psilocybin mushrooms which primarily grow on the ground in meadows and woods of the subtropics and tropics, usually in soils rich in humus and plant debris, Hericium erinaceus (Lion's Mane mushrooms) grow on the bark of trees in temperate forests of the Northern United States and Canada, where they are able to withstand cold temperatures and frost. Further, psilocybin mushrooms are terrestrial, whereas Lion's Mane mushrooms are non-terrestrial (i.e., they grow on trees). Therefore, Lion's Mane mushrooms and psilocybin mushrooms live in different habitats and neither is found cohabitating or combined in nature.

As used herein, the term “tryptamine” refers to any compound related to or derived from the monoamine alkaloid 2-(1H-Indo)-3-yl)ethanamine (tryptamine), a non-selective 5-HT_{2A} agonist and serotonin-norepinephrine-dopamine releasing agent (SNDRA). The tryptamine may be a natural product extracted from or isolated from a natural source, such as a Psilocybe mushroom, or synthesized synthetically. Exemplary tryptamines include psilocybin, baeocystin, norbaeocystin, psilocin, norpsilocin, 4-hydroxytryptamine, N,N-dimethyltryptamine (DMT), 5-hydroxytryptamine (serotonin), tryptamine, N-methyltryptamine, N-methyltryptamine, inter alia, pharmaceutically acceptable salts, hydrates, solvates, prodrugs, synthetics, analogs, congeners, isomers, stereoisomers, or tautomers thereof. Tryptamines such as psilocybin, psilocin, and baeocystin when in mushrooms from nature are known to decay over time, especially quickly in suboptimal storage conditions. Repke et al., J. Pharmac. Sci. 66(1): 113-114 (1977). In another study, psilocybin content of P. cubensis ranged from 0.102% to 0.706%, while psilocin content ranged from 0.415% to 0.836% of dried mushroom tissue. Gambaro et al., J. Pharmac. Biomed. Anal. 125: 427-432 (2016). This variability is consistent with trends previously observed, where psilocybin content of mushroom tissue appeared to increase with subsequent flushes. Bigwood and Beug, J. Ethnopharmacology 5(3): 287-291 (1982); Beug amd Bigwood, J. Ethnopharmacology 5(3): 271-285 (1982). The growing substrate can affect the tryptamine concentration as well. Gartz found that growing Psilocybe cubensis on a cow dung-rice growing substrate increased psilocin content in Psilocybe cubensis from 0.09% to 3.3% of the dried mushroom weight. See Gartz, Planta Med. 55(3): 249-250 (1989). Also, different parts of a mushroom can have different quantities of these compounds. For example, one study found that psilocybin is highest in the caps of Panaeolus subalteatus as compared to the rest of the fruiting body. See Gartz, Biochemie and Physiologie der Pflanzen. 184(1-2): 171-178 (1989). In addition to psilocybin and psilocin, several other tryptamine alkaloid compounds can also be present in varying concentrations in mushrooms found in nature. Generally, these compounds are part of the same biosynthetic pathway that yields psilocybin and can be distinguished from one another based on the presence or absence of one or more methyl or phosphate groups. This unique class of biochemicals is referred to as psilocybin analogs. Few reports of baeocystin consumption exist. In a 1997 book, Jochen Gartz reported that baeocystin was roughly akin to psilocybin in terms of its potency and psychotropic effects. The same author had previously published an anecdotal experience where he experienced a “gentle hallucinogenic experience” after consuming 4 mg of baeocystin. See Gartz, Ann Mus civ Rovereto 7: 265-74 (1991). Additional case studies of oral consumption of 10 mg and 20 mg baeocystin did not produce any hallucinogenic effects. Even less is understood about norpsilocin, baeocystin's dephosphorylated derivative, which was only recently identified. Lenz et al., J. Nat. Prod. 80(10): 2835-2838 (2017).

The term “alkyl” refers to a radical of a straight chain or branched saturated hydrocarbon group having from 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tent-butyl, sec-butyl, isobutyl), pentyl (C₆) (e.g., n-pentyl, 3-pentanyl, amyl, neopentyl, 3-methyl-2-butanyl, tertiary amyl), and hexyl (C₆) (e.g., n-hexyl).

“Alkylene” refers to a divalent radical of an alkyl group, e.g., —CH₂—, —CH₂CH₂—, and —CH₂CH₂CH₂—.

“Heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and one or more heteroatoms within the parent chain (“heteroC₁₋₈ alkyl”). In some embodiments, a heteroalkyl group is a saturated group having 1 to 7 carbon atoms and one or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC1 alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

“Heteroalkylene” refers to a divalent radical of a heteroalkyl group.

“Alkoxy” or “alkoxyl” refers to an —O-alkyl radical. In some embodiments, the alkoxy groups are methoxy, ethoxy, n-propoxy, isopropoxy, n-butoxy, tert-butoxy, sec-butoxy, n-pentoxy, n-hexoxy, and 1,2-dimethylbutoxy. In some embodiments, alkoxy groups are lower alkoxy, i.e., with between 1 and 6 carbon atoms. In some embodiments, alkoxy groups have between 1 and 4 carbon atoms.

As used herein, the term “aryl” refers to a stable, aromatic, mono- or bicyclic ring radical having the specified number of ring carbon atoms. Examples of aryl groups include, but are not limited to, phenyl, 1-naphthyl, 2-naphthyl, and the like. The related term “aryl ring” likewise refers to a stable, aromatic, mono- or bicyclic ring having the specified number of ring carbon atoms.

As used herein, the term “heteroaryl” refers to a stable, aromatic, mono- or bicyclic ring radical having the specified number of ring atoms and comprising one or more heteroatoms individually selected from nitrogen, oxygen, and sulfur. The heteroaryl radical may be bonded via a carbon atom or heteroatom. Examples of heteroaryl groups include, but are not limited to, furyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl, pyridyl, quinolinyl, isoquinolinyl, indolyl, indazolyl, oxadiazolyl, benzothiazolyl, quinoxalinyl, and the like. The related term “heteroaryl ring” likewise refers to a stable, aromatic, mono- or bicyclic ring having the specified number of ring atoms and comprising one or more heteroatoms individually selected from nitrogen, oxygen, and sulfur.

As used herein, the term “carbocycle” refers to a stable, saturated, or unsaturated, non-aromatic, mono- or bicyclic (fused, bridged, or spiro) ring radical having the specified number of ring carbon atoms. Examples of carbocycle groups include, but are not limited to, the cycloalkyl groups identified above, cyclobutenyl, cyclopentenyl, cyclohexenyl, and the like. In an embodiment, the specified number is C₃-C₁₂ carbons. The related term “carbocyclic ring” likewise refers to a stable, saturated, or unsaturated, non-aromatic, mono- or bicyclic (fused, bridged, or spiro) ring having the specified number of ring carbon atoms.

As used herein, the term “heterocyclyl” refers to a stable, saturated or unsaturated, non-aromatic, mono- or bicyclic (fused, bridged, or spiro) ring radical having the specified number of ring atoms and comprising one or more heteroatoms individually selected from nitrogen, oxygen and sulfur. The heterocyclyl radical may be bonded via a carbon atom or heteroatom. In an embodiment, the specified number is C₃-C₁₂ carbons. Examples of heterocyclyl groups include, but are not limited to, azetidinyl, oxetanyl, pyrrolinyl, pyrrolidinyl, tetrahydrofuryl, tetrahydrothienyl, piperidyl, piperazinyl, tetrahydropyranyl, morpholinyl, perhydroazepinyl, tetrahydropyridinyl, tetrahydroazepinyl, octahydropyrrolopyrrolyl, and the like. The related term “heterocyclic ring” likewise refers to a stable, saturated or unsaturated, non-aromatic, mono- or bicyclic (fused, bridged, or spiro) ring having the specified number of ring atoms and comprising one or more heteroatoms individually selected from nitrogen, oxygen and sulfur.

As used herein, “spirocycloalkyl” or “spirocycle” means carbogenic bicyclic ring systems with both rings connected through a single atom. The rings can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. For example, a (C₃-C₁₂)spirocycloalkyl is a spirocycle containing between 3 and 12 carbon atoms.

As used herein, “spiroheterocycloalkyl” or “spiroheterocycle” means a spirocycle wherein at least one of the rings is a heterocycle wherein one or more of the carbon atoms can be substituted with a heteroatom (e.g., one or more of the carbon atoms can be substituted with a heteroatom in at least one of the rings). One or both of the rings in a spiroheterocycle can be fused to another ring carbocyclic, heterocyclic, aromatic, or heteroaromatic ring.

As used herein, “halo” or “halogen” refers to fluorine (fluoro, —F), chlorine (chloro, —Cl), bromine (bromo, —Br), or iodine (iodo, —I).

As used herein, “haloalkyl” means an alkyl group substituted with one or more halogens. Examples of haloalkyl groups include, but are not limited to, trifluoromethyl, difluoromethyl, pentafluoroethyl, and trichloromethyl.

As used herein, “substituted,” whether preceded by the term “optionally” or not, means that one or more hydrogens of the designated moiety are replaced with a suitable substituent.

As used herein, the definition of each expression, e.g., alkyl, m, n, etc., when it occurs more than once in any structure, is intended to be independent of its definition elsewhere in the same structure.

Definitions of specific functional groups and chemical terms are described in more detail herein. The chemical elements are identified in accordance with the Periodic Table of the Elements, CAS version, Handbook of Chemistry and Physics, 75^{th} ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} ed, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} ed, Cambridge University Press, Cambridge, 1987.

Certain compounds described herein may exist in particular geometric or stereoisomeric forms. A particular enantiomer of a compound described herein may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Unless otherwise stated, structures depicted herein are also meant to include geometric (or conformational) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the disclosed compounds are within the scope of the disclosure. Unless otherwise stated, all tautomeric forms of the compounds described herein are within the scope of the disclosure. Additionally, unless otherwise stated, structures depicted herein are also meant to include compounds that differ only in the presence of one or more isotopically enriched atoms. For example, compounds having the disclosed structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the disclosure.

The “enantiomeric excess” or “% enantiomeric excess” of a composition can be calculated using the equation shown below. In the example shown below a composition contains 90% of one enantiomer, e.g., the S enantiomer, and 10% of the other enantiomer, i.e., the R enantiomer. ee=(90−10)/100×100=80%.

Thus, a composition containing 90% of one enantiomer and 10% of the other enantiomer is said to have an enantiomeric excess of 80%. The compounds or compositions described herein may contain an enantiomeric excess of at least 50%, 75%, 90%, 95%, or 99% of one form of the compound, e.g., the S-enantiomer. In other words, such compounds or compositions contain an enantiomeric excess of the S enantiomer over the R enantiomer.

Where a particular enantiomer is preferred, it may, in some embodiments be provided substantially free of the corresponding enantiomer and may also be referred to as “optically enriched.” “Optically enriched,” as used herein, means that the compound is made up of a significantly greater proportion of one enantiomer. In certain embodiments, the compound is made up of at least about 90% by weight of a preferred enantiomer. In other embodiments, the compound is made up of at least about 95%, 98%, or 99% by weight of a preferred enantiomer. Preferred enantiomers may be isolated from racemic mixtures by any method known to those skilled in the art, including chiral high-pressure liquid chromatography (HPLC) and the formation and crystallization of chiral salts or prepared by asymmetric syntheses. See e.g., Jacques et al., Enantiomers, Racemates and Resolutions (Wiley Interscience, New York, 1981); Wilen, et al., Tetrahedron 33:2725 (1977); Eliel, E. L. Stereochemistry of Carbon Compounds (McGraw Hill, NY, 1962); Wilen, S. H. Tables of Resolving Agents and Optical Resolutions p. 268 (E. L. Eliel, Ed., Univ. of Notre Dame Press, Notre Dame, IN 1972).

Any resulting mixtures of isomers can be separated based on the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds described herein into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed.

Various exemplary embodiments of the disclosure are described herein. It will be recognized that features specified in each embodiment may be combined, substituted, or replaced with other specified features disclosed elsewhere in the specification to provide further embodiments of the present disclosure. All analogous compounds may be substituted for each other in the same or similar amounts (mass, concentration, or dosages) as indicated for analogous compounds.

It is understood that in the following embodiments, combinations of substituents or variables of the depicted formulae are permissible only if such combinations result in stable compounds.

Described herein are compositions and methods for treating and/or alleviating symptoms of adverse reactions, such as an increased anti-inflammatory response, triggered by infectious diseases or conditions. Current therapies for the treatment of increased anti-inflammatory responses, such as cytokine storm, aim to dampen the immune system response. The treatments include blocking specific cytokines, such as IL-6 with tocilizumab or siltuximab, and generalized immunosuppressive drugs, such as corticosteroids. However, immunosuppressive drugs are accompanied by many negative side effects such as increasing susceptibility to infections and can interfere with anti-cancer immunotherapies. Recently, COVI D-19 has been shown to cause cytokine storm and potentially cause death. Thus, there remains a need for alternative medicines that have decreased side effects and can be used in all patients. Polysaccharides in mushrooms have been shown to initiate an immune response, sparking activity of TNF-α, IL-1β, IL-6, and other pro-inflammatory proteins involved in acute immune activation. Concern has been raised regarding isolated polysaccharide extracts and induction of IL-1β, an inflammatory cytokine that may exacerbate the runaway inflammatory presentations in later stages of COVID-19. However, we have shown that other biologically active compounds in mushrooms and mycelium (such as the sterols, phenols, and other terpenoid compounds) are important for the resolution of this inflammatory response, inducing anti-inflammatory cytokines such as IL-10 and IL-1ra. Therefore, it is surprising that an unextracted whole mushroom mycelium complex impacts the immune system in a balanced and modulatory manner and may decrease cytokine storm.

### Cytokine Storm

The term “increased anti-inflammatory response” as used herein refers to an exacerbated immune response to an infection, therapeutic, or autologous or allogeneic cells and tissues. The increased anti-inflammatory response may be a dysregulated pro-inflammatory cytokine response to an infection, therapeutic, or autologous or allogeneic cells and tissues. The increased anti-inflammatory response includes the rapid release of pro-inflammatory and anti-inflammatory cytokines, where the release of these initial cytokines can lead to an inflammatory cascade. The increased anti-inflammatory response may be a cytokine storm.

As used herein, the terms “cytokine storm,” “cytokine release syndrome,” “macrophage activation syndrome,” and “hemophagocytic lymphohistiocytosis” interchangeably refer to the dysregulation of pro-inflammatory and anti-inflammatory cytokines leading to disease. A cytokine storm may be referred to as being part of a sequence because one cytokine typically leads to the production of multiple other cytokines that can reinforce and amplify the immune response. Cytokine storm is a potentially life-threatening cytokine-associated toxicity. Diagnosing and management of cytokine storm is routinely based on clinical parameters and symptoms, such as identifying biomarkers (e.g., gene products (e.g., polypeptides, gene expression and/or protein expression profiles), or other analytes). Cytokine storm results from high-level immune activation when large numbers of lymphocytes and/or myeloid cells release inflammatory cytokines upon activation. The severity of the cytokine storm and the timing of onset of symptoms can vary depending on the magnitude of immune cell activation. The pro-inflammatory mediators involved in cytokine storm are divided into two subgroups: early mediators and late mediators. The transcription factor interferon regulatory factor 5 (IRF5) is critical for pro-inflammatory cytokine production. The inflammatory response to influenza infection is known to increase glucose metabolism. Glucose metabolism is required for activating IRF5-induced cytokine production, specifically the hexosamine biosynthesis pathway. Hexosamine biosynthesis results in the end product uridine diphosphate N-acetylglucosamine (UDP-GlcNAc). Through O-GlcNAcylation, UDP-GlcNAc is added to proteins to modify their activity. It has been shown that O-GlcNAcylation of IRF5 is necessary for IRF5-mediated cytokine production. It also has been shown that influenza patients have higher blood glucose levels and more O-GlcNacylation of IRF5 than healthy controls and that blood glucose levels are highly correlated with levels of inflammatory cytokines. Therefore, glucose metabolism plays a role in the development of cytokine storm.

Disease conditions commonly associated with a cytokine storm include but are not limited to: sepsis, systemic inflammatory response syndrome (SIRS), cachexia, septic shock syndrome, traumatic brain injury (e.g., cerebral cytokine storm), graft versus host disease (GVHD), or the result of treatment with activated immune cells, e.g., IL-2 activated T cells, T cells activated with anti-CD19 Chimeric Antigen Receptor (CAR) T cells. Infectious diseases commonly associated with cytokine storm include viral, bacterial, and parasitic infections. The viral infectious diseases include, but are not limited to, Paramyxoviridae (respiratory syncytial virus (RSV), parainfluenza virus (PIV), metapneumovirus (MPV), enteroviruses), Picornaviridae (Rhinovirus, RV), Coronaviridae (CoV), Adenoviridae (Adenovirus), Parvoviridae (HBoV), Orthomyxoviridae (influenza A, B, C, D, Isavirus, Thogotovirus, Quaranjavirus), Herpesviridae (human herpes viruses, Varicella zoster virus, Epstein-Barr virus, cytomegalovirus), avian influenza, smallpox, pandemic influenza, adult respiratory distress syndrome (ARDS). CoV can include one or more of Severe Acute Respiratory Syndrome (SARS-CoV), Middle East Respiratory Syndrome (MERS-CoV), COVID-19 (2019-nCoV, SARS-CoV-2), 229E, NL63, O043, or HKU1. The bacterial infectious diseases include, but are not limited to, Streptococcus pneumoniae, Mycobacterium tuberculosis, Bordetella pertussis, Haemophilus influenzae, Moraxella catarrhalis, Pseudomonas aeruginosa, Stenotrophomonas maltophila, Staphylococcus aureus, Streptococcus pyogenes, Neisseria meningitidis, Klebsiella pneumoniae, or Non-tuberculosis Mycobacterium. The parasitic infectious diseases include, but are not limited to, malaria.

Coronaviruses (CoVs), are enveloped positive-sense RNA viruses, which are surrounded by crown-shaped, club-like spike projections on the outer surface. Coronaviruses' spike proteins are glycoproteins that are embedded over the viral envelope. This spike protein attaches to specific cellular receptors and initiates structural changes of the spike protein, and causes penetration of cell membranes, which results in the release of the viral nucleocapsid into the cell. These spike proteins determine host trophism. Coronaviruses have a large RNA genome, ranging in size from 26 to 32 kilobases and capable of obtaining distinct ways of replication. Like other RNA viruses, coronaviruses under-go replication of the genome and transcription of mRNAs upon infection. Coronavirus infection in a subject can result in significant and long-term damage of the lungs, leading to possibly severe respiratory issues.

As used herein “2019-nCoV” is a betacoronavirus (Beta-CoV or β-CoV). In particular, 2019-nCoV is a Beta-CoV of lineage B. 2019-nCoV may also be known as SARS-CoV-2 or 2019 novel coronavirus. Betacoronaviruses are one of four genera of coronaviruses and are enveloped, positive-sense, single-stranded RNA viruses of zoonotic origin. Betacoronaviruses mainly infect bats, but they also infect other species like humans, camels, and rabbits. 2019-nCoV may be transferable between animals, such as between humans. As used herein, “viral transmission” is the process by which viruses spread between host subjects. Transmission occurs from person to person by direct or indirect contact or exposure. Examples of direct contact include, but are not limited to, the exchange of body fluids between a subject infected with the virus and someone else. Indirect contact includes, but is not limited to, exposure to bodily fluid droplets produced by a subject infected by the virus during coughing and/or sneezing. Beta-CoVs may induce fever and respiratory symptoms in humans. The overall structure of β-CoV genome contains an ORF1ab replicase polyprotein (rep, pplab) preceding other elements. This polyprotein is cleaved into many nonstructural proteins. 2019-nCoV has a phenylalanine in the (F486) in the flexible loop of the receptor binding domain, flexible glycyl residues, and a four amino acid insertion at the boundary between the S1 and S2 subunits that results in the introduction of a furin cleavage site. The furin cleavage site may result in 2019-nCoV tissue tropism, increase transmissibility, and alter pathogenicity.

Diagnosis of 2019-nCoV may comprise a positive test for 2019-nCoV and/or onset of 2019-nCoV symptoms, or combinations thereof. Symptoms of 2019-nCoV include, but are not limited to, one or more of the following symptoms: nasal congestion, sore throat, fever, body aches, exhaustion, dry cough, difficulty breathing, loss of taste, loss of smell, or a combination thereof. The methods and compositions herein can recover or aid in the recovery of taste and smell. Subjects may also experience long-term effects from COVID-19, sometimes referred to as “long COVID”, where symptoms can persist for weeks or months after the initial infection and disappear and reappear after infection. Long-term COVID-19 symptoms include, but are not limited to, one or more of shortness of breath, cough, fatigue, joint pain, chest pain, difficulty with thinking and/or concentration (i.e. “brain fog”), depression, anxiety, changes in mood, muscle pain, headache, intermittent fever, heart palpitations, inflammation of the heart, lung function abnormalities, acute kidney injury, rash, hair loss, smell and/or taste problems, sleep issues, and difficulty with memory. Subjects who experience long-term effects from COVID-19 are known as long-haulers. The methods and compositions herein may treat long-term COVID-19 or decrease the symptoms thereof. Subjects at higher risk of developing complications may be immunocompromised (e.g., undergoing cancer treatment, bone marrow or organ transplantation, immune deficiencies, poorly controlled HIV or AIDS, prolonged use of corticosteroids or immune weakening medications), have an underlying medical condition (e.g., diabetes, renal failure, liver disease), are pregnant, are at least 65 years of age, have a chronic lung disease, have a heart disease, or combinations thereof.

Symptoms of cytokine storm can include neurologic toxicity, disseminated intravascular coagulation, cardiac dysfunction, adult respiratory distress syndrome, renal failure, and/or hepatic failure. For example, symptoms of cytokine storm can include fever with or without rigors, fatigue, malaise, myalgias, vomiting, headache, nausea, anorexia, arthralgias, diarrhea, rash, hypoxemia, tachypnea, hypotension, widened pulse pressure, potentially diminished cardiac output (late), increased cardiac output (early), azotemia, hypofibrinogenemia with or without bleeding, elevated D-dimer, hyperbilirubinemia, transaminitis, confusion, delirium, mental status changes, hallucinations, tremor, seizures, altered gait, word finding difficulty, frank aphasia, elevated heart rate, coagulopathy, MODS (multiple organ dysfunction syndrome), cardiovascular dysfunction, distributive shock, cardiomyopathy, hepatic dysfunction, renal dysfunction, encephalopathy, clinical seizures, respiratory failure, tachycardia, or dysmetria.

IL-6 is thought to be a mediator of cytokine storm toxicity. High IL-6 levels may initiate a pro-inflammatory IL-6 signaling cascade, leading to one or more of the cytokine storm symptoms. IL-6 and soluble IL-6 receptor (sIL-6R) levels can be measured for example, by methods described in Chen et al., J. Immunol. Meth. 434:1-8 (2016). In some cases, the level of C-reactive protein (CRP) (a biomolecule produced by the liver, e.g., in response to IL-6) can be a measure of IL-6 activity. In some cases, CRP levels may increase several-fold (e.g., several logs or orders of magnitude) during cytokine storm. CRP levels can be measured using standard methods available in the art. Spiking IL-6 is dangerous for COVID-19 as it potentiates a cytokine storm, therefore current clinical trials aim to suppress IL-6 in COVID-19 patients. The compositions described herein spike IL-6 while simultaneously spiking IL-4, IL-10, and/or IL-1 receptor antagonist (IL-1RA), and therefore surprisingly reduces cytokine storms, neuroinflammation, and blood clotting.

Rapidly proliferating and highly activated T-cells or natural killer (NK) cells that result in the exaggerated release of cytokines during a cytokine storm can include more than 150 inflammatory mediators such as cytokines, oxygen free radicals, and coagulation factors. Both pro-inflammatory cytokines (such as TNF-α, IL-1, and IL-6) and anti-inflammatory cytokines (such as IL-10, IL-4, and IL-1RA become greatly elevated in, for example, serum. It is this excessive release of inflammatory mediators that triggers the cytokine storm.

A “pro-inflammatory cytokine” or a “pro-inflammatory mediator” is an immuno-regulatory cytokine that induces inflammation. A pro-inflammatory cytokine may upregulate or increase the synthesis of secondary pro-inflammatory mediators and other pro-inflammatory cytokines by immune cells. In addition, pro-inflammatory cytokines can stimulate production of acute phase proteins that mediate inflammation and attract inflammatory cells. Pro-inflammatory cytokines that are generally responsible for early immune responses include IL-1, IL-6, and TNF-α. IL-1, IL-6, and TNF-α are also considered endogenous pyrogens as they contribute to increasing body temperature. Other examples of pro-inflammatory cytokines or pro-inflammatory mediators include IL-2, IL-4, IL-5, IL-6, IL-7, IL-8, IL-9, IL-11, IL-12p70, IL-13, IL-15, IL-17A, IL-18, interferon (IFN)-γ, monocyte chemoattractant protein (MCP) 1, eotaxin, interferon gamma-induced protein (IP) 10, granulocyte colony-stimulating factor (GM-CSF), macrophage inflammatory protein (MIP) 1α, MIP 1β, RANTES, leukemia, inhibitory factors (LIF), oncostatin M (OSM), and a variety of chemokines that attract inflammatory cells.

IL-1 is an important pro-inflammatory cytokine. IL-1 is a soluble protein having a mass of approximately 17 kilo-Daltons (kD). IL-1 is produced by a variety of cells, for example macrophages, white blood cells, lymphocytes, monocytes, dendritic cells, and accessory cells that are involved in activation of T-lymphocytes and B-lymphocytes. IL-1 is typically released by such cells during an immune response. IL-1 is generally considered to be a pro-inflammatory cytokine. The original members of the IL-1 superfamily are IL-1α, IL-β, and IL-1 receptor antagonist (IL-1RA). Both IL-1α and IL-1β play important roles in the inflammatory response of the body against pathogens or infection and recognize the same IL-1 receptor and perform similar biological functions. IL-1α is predominantly a cell-associated molecule whereas IL-1β is generally a secreted molecule. The term “IL-1” as used herein includes one or both of IL-1α and IL-1β. IL-1 can increase the expression of adhesion factors on endothelial cells to enable transmigration of leukocytes to sites of infection. In addition, IL-1 can stimulate the hypothalamus thermoregulatory center to cause an increase in body temperature (e.g., a fever). In particular, IL-1β is involved in a range of cellular activities such as cell proliferation, cell differentiation, cell apoptosis, and pain. TNF-α is involved in systemic inflammation and works in tandem with a variety of other cytokines to stimulate the acute phase immune reaction. TNF-α can induce apoptotic cell death as well as inhibit tumorigenesis and viral replication. TNF-α and IL-1 can work simultaneously and synergistically in stimulating and sustaining inflammation within the body.

“Anti-inflammatory cytokines” or “anti-inflammatory mediators” refer generally to immuno-regulatory cytokines that inhibit or counteract various aspects of inflammation. In other words, anti-inflammatory cytokines counteract various biological effects of pro-inflammatory cytokines and pro-inflammatory mediators. Anti-inflammatory cytokines can control or mitigate the magnitude of inflammation. Functions of anti-inflammatory cytokines include inhibiting production of pro-inflammatory cytokines and inhibiting cell activation. Examples of anti-inflammatory cytokines include, but are not limited to, IL-1RA, IL-2, IL-4, IL-7, IL-9, IL-10, IL-13, or IL-15. IL-2 is a variably glycosylated single protein molecule having as mass of approximately 15.5 kD. IL-2 is generally produced by activated T helper cells (also known as effector T cells) during an immune response. Pathogens (also known as antigens) that invade or are introduced within the body bind to receptors that are found on the surfaces of lymphocytes. Binding of such pathogens or antigens to T cell receptors (TCR) stimulates secretion of IL-2. IL-2 mediates its effects by binding to IL-2 receptor molecules, which are expressed by lymphocytes. The binding of IL-2 to its receptor molecule triggers a signaling cascade, for example Ras/MAPK, JAK/Stat, and PI 3-kinase/Akt signaling modules. IL-2 has numerous functions including facilitating production of immunoglobulins (Ig) by B cells. In addition, IL-2 induces differentiation and proliferation of NK cells and stimulates growth, differentiation, and proliferation of antigen-selected cytotoxic T cells via induction gene expression. IL-2 is considered to be important for the development of T cell immunologic memory and is necessary during T cell development in the thymus for enabling the maturation of regulatory T cells.

Given the mutability of viruses as they jump from host to host, many variants of viruses can evolve and emerge, with some becoming more damaging and deadlier. Although vaccines and antiviral drugs can be effective against one strain of virus when they are designed and tested, these continuous mutations can result in vaccine evasion or loss in drug potency. Viruses or other pathogens that can evade the efficacy of vaccines and drugs, make these disease agents more virulent, more contagious, and ultimately more deadly to those infected. Hence, by augmenting immunity while downregulating specific cytokines can help not only lessen the degree of infection but also prevent cytokine storms, making vaccines, anti-viral drugs, and other treatments more effective. The methods and compositions described herein, in conjunction with conventional therapies such as vaccines and antiviral drugs, may potentiate the recovery of patients infected with viruses such as COVID, flu viruses and other known viruses, due to the immune enhancement properties coupled with the anti-inflammatory effects of the methods and compositions described herein, and the stimulation of IL-10, IL1 RA, and/or IL-4 by the methods and compositions described herein. Moreover, the use of the methods and compositions as described herein can help enhance innate immunity of patients who suffer from viruses that are immune evasive. Examples of immune evasion include but are not limited to cancer-causing viruses (oncoviruses) that are responsible for Merkel Cell Carcinoma, and the many forms of human papillomavirus (HPV) and Herpes induced cancers. In essence, these mushroom adjuvant therapies described herein can enhance innate immune system, helping decloak or sensitize these disease agents and in some cases the cancers they cause, while augmenting better immune detection, targeting, and vaccine/drug efficacy-enhancing recovery. The methods and compositions herein can enhance a wide range of conventional therapies currently in practice for fighting viral and microbial diseases. Conventional viral therapies include, but are not limited to, vaccines such as live attenuated virus vaccines, attenuated virus vaccines, mRNA vaccines, cell-based vaccines, recombinant vaccines, adjuvant vaccines, or quadrivalent vaccines; and antiviral drugs such as oseltamivir (Tamiflu), zanamivir (Relenza), peramivir (Rapivab) or baloxavir (Xofluza).

### Monoamine Oxidase Inhibitors

Monoamine oxidase inhibitors (MAOls) target two different isoenzymes of monoamine oxidase (MAO-A and MAO-B). MAOls can be nonselective or selective for these isoenzymes and there is a subclass MAOI that is reversible and specific to MAO-A (RIMA). MAOls were the first class of antidepressants to be developed and the FDA has approved several as depression treatments, including phenelzine, tranylcypromine, selegiline, and isocarboxazid. In addition to atypical or treatment-resistant depressions, MAOls have been used to treat Parkinson's disease, as well as anxiety, post-traumatic stress, panic, bipolar, and obsessive-compulsive disorders. Among MAOls are a class of compounds called β-carbolines, a group of indole alkaloids derived from tryptophan that are naturally produced by bacteria, plants, animals, and fungi.

The two most commonly cited β-carbolines are harmane (also harman) and norharmane (also norharman) (Piechowska et al., Nutrients 11(4): 814 (2019)). Harmine and harmaline are also frequently identified derivatives of the norharmane (sometimes referred to generically as β-carboline) backbone. Some molecules, like tetrahydro-β-carbolines are precursors to these aromatic β-carboline molecules. Harmine and harmaline both function as specific MAO-A inhibitors, being poor inhibitors of MAO-B (Herraiz et al., Food Chem. Tox. 48(3): 839-845 (2010)). Harmine seems to have a stronger inhibitory effect, as demonstrated by a comparison of the inhibitory effects of the plant and seed fractions of Peganum harmala, aka Syrian rue or wild rue, which endogenously creates both compounds. Seed extracts, whose effects were attributed to both harmaline and harmine, had an IC₅₀ of 27 ug/L when inhibiting MAO-A (Herraiz et al., 2010). Comparatively, root extracts, whose effects were attributed to solely harmine, had an IC₅₀ of 159 ug/L.

β-carbolines are found in a wide variety of plants, fungi, bacteria, and animals. Well known plants such as Syrian Rue (Peganum harmala), Soul Vine (Banisteriopsis caapi), Passionflower (Passiflora incarnata), Tobacco (Nicotiana tabacum), and Coffee (Caffea arabica) contain alkaloids such as harmane, harmine, telepathine, and banisterine, among others (Piechowska et al., Nutrients 11(4): 814 (2019)). In fungi, a number of species contain β-carbolines from a wide variety of taxa. Entomopathogenic fungi such as Ophiocordyceps sinensis and Conidiobolus coronatus are known to contain β-carbolines such as the cordysinins and harmane, respectively. Other higher order fungi also produce β-carbolines. It has been detected in carpophores of a number of Psilocybe species, as well as in Amanita muscaria and pantherina (Blei et al., Chem. Eur. J. 26(3), 729-734 (2020); Yang et al., J. Nat. Prod. 74(9): 1996-2000 (2011); Tsujikawa et al., Forensic Sci. Int 164(2-3), 172-178 (2006); Wronska et al., PLOS One 13(10): e0204828 (2018)). In bacteria, particularly soil dwelling Streptomyces species, β-carbolines may be used as extracellular signaling molecules, that in populations of bacteria encourage the biosynthesis of certain secondary metabolites (Panthee et al., Scientific Rep. 10(1) (2020)). β-carbolines are also found in some animal sources. The fluorescent activity under blacklight of the exoskeleton of scorpions is thought to be due to the β-carboline content of the cuticle. Although it is currently the subject of some scientific dispute, pinoline was long thought to be a β-carboline endogenously produced in the human pineal gland (Barker et al., Biomed. Chromat. 27(12), 1690-1700 (2013)). This compound was found to have neurogenic activity in an in vitro rat stem cell model at very low doses (de la Fuente Revenga et al., ACS Chem. Neurosci. 6(5), 800-810 (2015)).

The β-carboline harmine has traditionally been used for a drug-liberating effect during the preparation of ayahuasca, a pan-South American psychoactive drink created by combining two species of plants, one containing DMT, and one containing β-carbolines, specifically harmine. Because DMT is usually enzymatically deactivated when taken orally, the inclusion of harmine allows it to pass undestroyed into the brain, where it can take effect. This combination of a MAO inhibitor and psychoactive compound has been called “the ayahuasca effect” (McKenna and Towers, J. Psychoactive Drugs 16(4): 347-358 (1984)). β-carbolines have been identified in Psilocybe spp. and may inhibit metabolism of the prodrug psilocin by MAO-A.

Harmane was identified as the most prevalent β-carboline in Psilocybe cubensis, with 16-fold higher concentrations in mycelium when compared to mushrooms (Blei et al., Chem. Eur. J. 26(3), 729-734 (2020)). Specifically, Blei et al. quantified carpophores of P. cubensis as having harmine concentrations 0.10 μg/g dried biomass, whereas mycelium contained 0.08 μg/g; whereas harmane was detected at 0.08 μg/g in carpophores and 1.32 μg/g in mycelium (Table 1); Blei et al., Chem. Eur. J. 26(3), 729-734 (2020). Imaging with MALDI-MS suggested that the highest concentrations of harmane were present near the leading edge of mycelium colony. Table 2, shown below, lists β-carbolines and related molecules putatively detected in extracts of Hericium erinaceus mycelium using mass spectroscopy.

TABLE 1
β-carboline Concentrations in Psilocybe cubensis
and P. Mexicana (μg/g dry mass)

| | |
|---|---|
| P. cubensis | P. Mexicana |

Harmane

| | | |
|---|---|---|
| Carpophores | 0.08 | n.d. |
| Mycelium | 1.32 | 21.48 |
| Sclerotia | — | 2.01 |

Harmine

| | | |
|---|---|---|
| Carpophores | 0.10 | 0.04 |
| Mycelium | 0.08 | 0.61 |
| Sclerotia | — | 1.62 |

TABLE 2
β-carbolines and related molecules putatively detected in extracts
of Hericium erinaceus mycelium using mass spectroscopy.

| Compound Name | Formula | Mass (Da) | Certainty* | Ions |
|---|---|---|---|---|
| Norharman | C₁₁H₈N₂ | 168.07 | High | +/− |
| Harmine | C₁₃H₁₂N₂O | 212.10 | High | +/− |
| 1,2,3,4-tetrahydro-β-carboline-3- | C₁₂H₁₂N₂O₂ | 216.09 | High | +/− |
| carboxylic acid | | | | |
| 1-methyl-1,2,3,4-tetrahydro-β-carboline- | C₁₃H₁₄N₂O₂ | 230.11 | High | +/− |
| 3-carboxylic acid | | | | |
| 1-methyl-2,3,4,9-tetrahydro-1H-β- | C₁₄H₁₄N₂O₄ | 274.10 | High | +/− |
| carboline-1,3-dicarboxylic acid | | | | |
| Harmaline | C₁₃H₁₄N₂O | 214.11 | Medium | + |
| N-methoxy-1-vinyl-β-carboline | C₁₄H₁₂N₂O | 224.10 | Medium | + |
| Ethyl 9H-β-arboline-3-carboxylate | C₁₄H₁₂N₂O₂ | 240.09 | Medium | + |
| 1-furyl-β-carboline-3-carboxylic acid | C₁₆H₁₀N₂O₃ | 278.07 | Medium | − |
| 1-[5-(methoxymethyl)-2-furyl]-9H-β- | C₁₈H₁₄N₂O₄ | 322.09 | Medium | − |
| carboline-3-carboxylic acid | | | | |
| 6-hydroxy-3-(6-hydroxy-1H-indol-3-yl)- | C₂₀H₁₃N₃O₄ | 359.09 | Low | − |
| 9H-β-carboline-4-carboxylic acid | | | | |
| Strictosidine | C₂₇H₃₄N₂O₉ | 530.23 | Low | − |
| (1S)-1-{[(2S,3R,4S)-2-(β--L- | C₂₈H₃₄N₂O₁₁ | 574.22 | Low | − |
| glucopyranosyloxy)-5- | | | | |
| (methoxycarbonyl)-3-vinyl-3,4-dihydro- | | | | |
| 2H-pyran-4-yl]methyl}-2,3,4,9- | | | | |
| tetrahydro-1H-β-carboline-1,3- | | | | |
| dicarboxylic acid | | | | |

*Certainty is described as High if both positive and negative ions featured matching retention times, Medium if two or more annotation databases showed matches, and Low if only the compound was annotated using a single database.

Based on the concentrations recorded in Blei et al., Chem. Eur. J. 26(3), 729-734 (2020), a 1 g dried dose P. cubensis mushrooms would only contain 0.00008 mg of harmane and 0.0001 mg of harmine. Given an expected effective MAOI dose of 70-200 mg, the naturally occurring β-carboline content (combination of harmane and harmine) in P. cubensis is negligible, amounting to less than 0.0003% of the required amount for MAOI activity. If the same calculations are performed using the numbers listed in the “Results and Discussion” section of Blei et al., then the maximum observed concentration of harmane observed in dried Psilocybe tissue is 21.48 μg/g, or 0.02148 mg harmane per gram of dried biomass. In this case, harmane would constitute just 0.031% of the requisite 70 mg MAOI content.

In another embodiment, the carboline comprises a compound having the structure of:

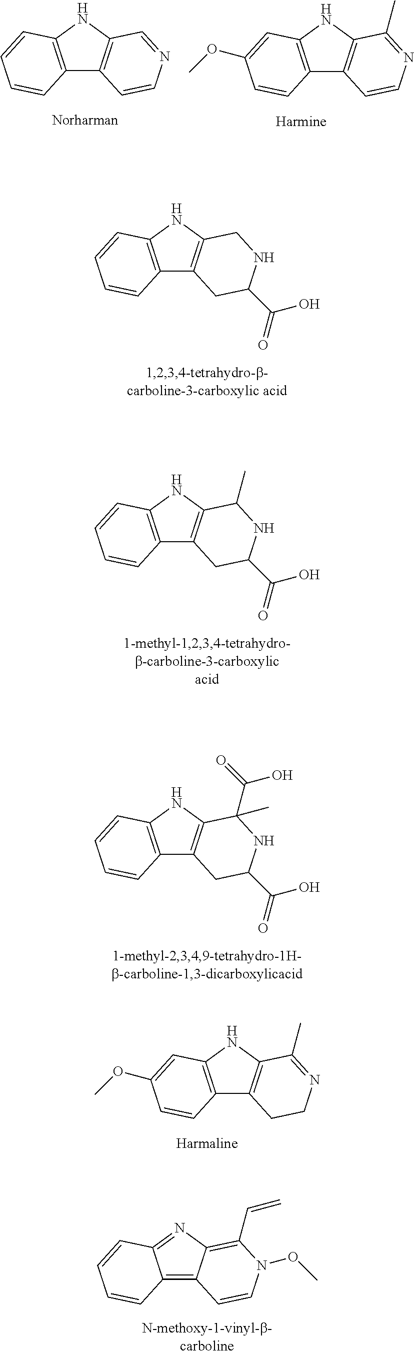

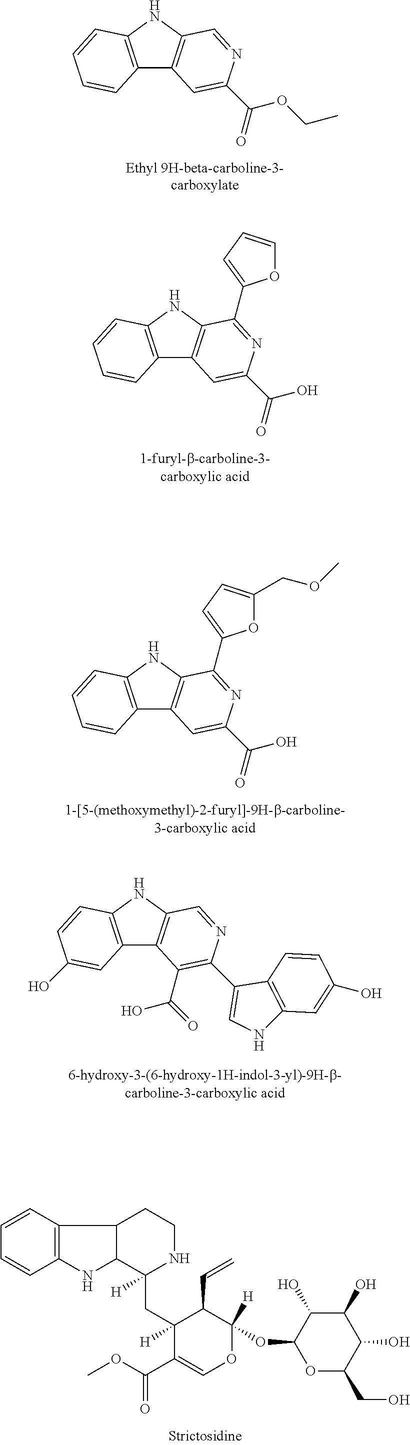 [SMILES: CCOC(=O)c1cc2c(cn1)[nH]c1ccccc12 ; O=C(O)c1cc2c([nH]c3ccccc32)c(-c2ccco2)n1 ; COCc1ccc(-c2nc(C(=O)O)cc3c2[nH]c2ccccc23)o1 ; O=C(O)c1c(-c2c[nH]c3cc(O)ccc23)ncc2[nH]c3ccc(O)cc3c12 ; C=C[C@H]1[C@H](O[C@@H]2O[C@H](CO)[C@@H](O)[C@H](O)[C@H]2O)OC=C(C(=O)OC)[C@H]1C[C@@H]1NCCC2c3ccccc3NC21]

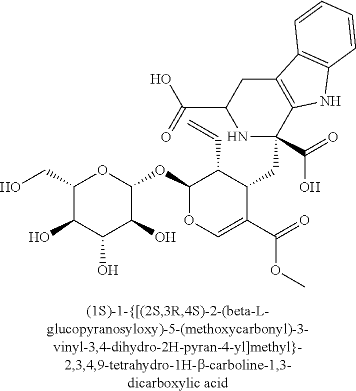 [SMILES: C=C[C@H]1[C@H](O[C@H]2O[C@@H](CO)[C@H](O)[C@@H](O)[C@@H]2O)OC=C(C(=O)OC)[C@H]1C[C@]1(C(=O)O)NC(C(=O)O)Cc2c1[nH]c1ccccc21]

Natural sources of non-β-carboline MAOIs include Areca catechu, Arisaema amurense, Calluna vulgari, Calytia japonica, Chelidonium majus (Chelerythine) (Baek et al., Bioorgan. Med. Chem. Let. 28(14), 2403-2407 (2018)), Chironia krebsii, Coptis chinensis, Curcuma longa (Cucurmin) (Juvekar et al., Pharmacognosy Magazine 12(46): 116 (2016)), Dictamnus albus, Garcinia gerrardii, Garcinia livingstonei, Geijera parvifiora, Gentiana lutea, Ginkgo biloba, Glycyrrhiza glabra (Isoliquiritigenin, Liquiritigenin), Halenia campanulate, Hypericum aucheri, Hipericum Brasiliense, Hypericum caprifoliatum, Hypericum hircinum, Hypericum piriai, Hypericum polyanthemum, Lilium brownie, Lycium chinense, Melissa officinalis, Mentha aquatic, Monascus anka, Monnina obtusifolia, Monnina sylvatica, Morinda citrifolia, Myristica fragrans (Kampferol, Myristicin) (Gidaro et al., J. Agri. Food Chem. 64(6): 1394-1400 (2016); Truitt, Psychopharmacol. Bul. 4(3): 14 (1967)), Opuntia ficus-indica var. saboten, Origanum vulgare (Oregano extract) (Mechan et al., Brit. J. Nutrition 105(8): 1150-1163 (2010)), Paeonia suffruticosa, Pentadesma reyndersii, Piper nigrum (Piperine), Polygala virgate, Polygonum multiflorum, Psoralea corylifolia, Rhazya stricta, Rhodiola rosea (van Diermen et al., J. Ethnopharmacology 122(2): 397-401 (2009)), Salvia miltiorrhiza, Sinofranchetia chinensis (Isoliquiritigenin, Liquiritigenin), Theobroma cacao (caffeine, catechin, epicatechin), Uncaria rhynchophylla, Vaccinum myrtillus, Zanthoxylum rigidum (Plazas et al., Bioorgan. Chem. 98: 103722 (2020)), Zanthoxylum schinifolium, Zingiber officinale (Geraniol from rhizomes).

### Compounds

In one embodiment described herein, the composition comprises one or more fungal compounds including tryptamines, or an amount of a mushroom (or plant) extract or mushroom (or plant) having an equivalent amount of tryptamine(s), or a combination thereof.

In one embodiment, the tryptamine comprises psilocybin, baeocystin, norbaeocystin, psilocin, norpsilocin, 4-hydroxytryptamine, N,N-dimethyltryptamine (DMT), 5-hydroxytryptamine (serotonin), tryptamine, aeruginascin, 4-hydroxy-N,N,N-trimethyltryptamine, 5-hydroxy-N,N,N-trimethyltryptamine (bufotenidine), N-methyltryptamine, N-ethyltryptamine, N-methyl-N-ethyltryptamine, N-methyl-N-propyltryptamine, N,N-diethyltryptamine, N-methyl-N-isopropyltryptamine, N-ethyl-N-isopropyltryptamine, N,N-diisopropyltryptamine, N,N-dipropyltryptamine, N,N-dipropyltryptamine, N,N-diallyltryptamine, 4-hydroxytryptamine, 4-hydroxy-N-methyltryptamine (norpsilocin), 4-hydroxy-N,N-dimethyltryptamine (psilocin), 4-hydroxy-N-methyl-N-ethyltryptamine, 4-hydroxy-N-methyl-N-propyltryptamine, 4-hydroxy-N,N-diethyltryptamine, 4-hydroxy-N,N-diethyltryptamine, 4-hydroxy-N-ethyl-N-isopropyltryptamine, 4-hydroxy-N,N-diisopropyltryptamine, 4-hydroxy-N,N-dipropyltryptamine, 4-hydroxy-N-N-dipropyltryptamine, 4-hydroxy-N,N-diallyltryptamine, 4-methoxytryptamine, 4-methoxy-N-methyltryptamine (norpsilocin), 4-methoxy-N,N-dimethyltryptamine (psilocin), 4-methoxy-N-methyl-N-ethyltryptamine, 4-methoxy-N-methyl-N-propyltryptamine, 4-methoxy-N,N-diethyltryptamine, 4-methoxy-N,N-diethyltryptamine, 4-methoxy-N-ethyl-N-isopropyltryptamine, 4-methoxy-N,N-diisopropyltryptamine, 4-methoxy-N,N-dipropyltryptamine, 4-methoxy-N-N-dipropyltryptamine, 4-methoxy-N-N-diallyltryptamine, 4-acetoxytryptamine, 4-acetoxy-N-methyltryptamine, 4-acetoxy-N-methyl-N-ethyltryptamine, 4-acetoxy-N-methyl-N-ethyltryptamine, 4-acetoxy-N-methyl-N-propyltryptamine, 4-acetoxy-N,N-diethyltryptamine, 4-acetoxy-N-methyl-N-isopropyltryptamine, 4-acetoxy-N-ethyl-N-isopropyltryptamine, 4-acetoxy-N,N-diisopropyltryptamine, 4-acetoxy-N,N-dipropyltryptamine, 4-acetoxy-N-N-dipropyltryptamine, 4-acetoxy-N-N-diallyltryptamine, 5-hydroxytryptamine, 5-hydroxy-N-methyltryptamine, 5-hydroxy-N,N-dimethyltryptamine (bufotenine), 5-hydroxy-N-methyl-N-ethyltryptamine, 5-hydroxy-N-methyl-N-propyltryptamine, 5-hydroxy-N,N-diethyltryptamine, 5-hydroxy-N-methyl-N-isopropyltryptamine, 5-hydroxy-N-ethyl-N-isopropyltryptamine, 5-hydroxy-N,N-diisopropyltryptamine, 5-hydroxy-N,N-dipropyltryptamine, 5-hydroxy-N-N-dipropyltryptamine, 5-hydroxy-N-N-diallyltryptamine, 5-methoxytryptamine, 5-methoxy-N-methyltryptamine, 5-methoxy-N,N-dimethyltryptamine, 5-methoxy-N-methyl-N-ethyltryptamine, 5-mehoxy-N-methyl-N-propyltryptamine, 5-methoxy-N,N-diethyltryptamine, 5-methoxy-N-methyl-N-isopropyltryptamine, 5-methoxy-N-ethyl-N-isopropyltryptamine, 5-methoxy-N,N-diisopropyltryptamine, 5-methoxy-N,N-dipropyltryptamine, 5-methoxy-N-N-dipropyltryptamine, 5-methoxy-N-N-diallyltryptamine, 5-acetoxytryptamine, 5-acetoxy-N-methyltryptamine, 5-acetoxy-N,N-dimethyltryptamine, 5-acetoxy-N-methyl-N-ethyltryptamine, 5-mehoxy-N-methyl-N-propyltryptamine, 5-acetoxy-N,N-diethyltryptamine, 5-acetoxy-N-methyl-N-isopropyltryptamine, 5-acetoxy-N-ethyl-N-isopropyltryptamine, 5-acetoxy-N,N-diisopropyltryptamine, 5-acetoxy-N,N-dipropyltryptamine, 5-acetoxy-N-N-dipropyltryptamine, 5-acetoxy-N-N-diallyltryptamine, α-methyltryptamine, N-ethyl-N-isopropyltryptamine, N-methyl-N-butyltryptamine, 2,α-dimethyltryptamine, α-N-dimethyltryptamine, α-methyl-N,N-dimethyltryptamine, α-ethyltryptamine, 2-methyl-N,N-dimethyltryptamine, 2-methyl-N,N-diethyltryptamine, 1-methylpsilocin, 5-methoxy-α-methyltryptamine, ibogaine, harmaline, 7-methoxy-1-methyl-1,2,3,4-tetrahydro-b-carboline (tetrahydroharmine), N,N-diethyl-D-lysergamide (LSD), 6-ally-N,N-diethyl-norlysergic acid (6-allyl-N,N-diethyl-norlysergic acid), 9,10-didehydro-N,N,6-triethylergoline-8b-carboxamide (6,N,N-triethyl-norlysergic acid), 9,10-didehydro-6-propyl-N,N-diethylergoline-8b-carboxamide (6-propyl-norlysergic acid), other tryptamine compounds, or a pharmaceutically acceptable salt, hydrate, solvate, prodrug, synthetic, analog, isomer, stereoisomer, congener, or tautomer thereof, or a combination thereof.

In another embodiment, the tryptamine comprises: 6-Allyl-N,N-diethyl-norlysergic acid (AL-LAD), N,N-dibutyl-tryptamine (DBT), N,N-diethyl-tryptamine (DET), N,N-diisopropyl-tryptamine (DiPT), 5-methyoxy-α-methyl-tryptamine (α,O-DMS), N,N-dimethyl-tryptamine (DMT), 2,α-dimethyl-tryptamine (2,α-DMT), α,N-dimethyl-tryptamine (α,N-DMT), N,N-dipropyl-tryptamine (DPT), N-ethyl-N-isopropyl-tryptamine (EiPT), α-ethyl-tryptamine (AET), 6,N,N-tryptamineriethyl-norlysergic acid (ETH-LAD), 3,4-dihydro-7-methoxy-1-methyl-carboline (Harmaline), 7-methyoxy-1-methyl-carboline (Harmine), N,N-dibutyl-4-hydroxy-tryptamine (4-HO-DBT), N,N-diethyl-4-hydroxy-tryptamine (4-HO-DET), N,N-diisopropyl-4-hydroxy-tryptamine (4-HO-DiPT), N,N-dimethyl-4-hydroxy-tryptamine (4-HO-DMT), N,N-dimethyl-5-hydroxy-tryptamine (5-HO-DMT), N,N-dipropyl-4-hydroxy-tryptamine (4-HO-DPT), N-ethyl-4-hydroxy-N-methyl-tryptamine (4-HO-MET), 4-hydroxy-N-isopropyl-N-methyl-tryptamine (4-HO-MiPT), 4-hydroxy-N-methyl-N-propyl-tryptamine (4-HO-MPT), 4-hydroxy-N,N-tetramethylene-tryptamine (4-HO-pyr-tryptamine), 12-methoxyibogamine (Ibogaine), N,N-diethyl-lysergic acid (LSD), N-butyl-N-methyl-tryptamine (MBT), N,N-diisopropyl-4,5-methylenedioxy-tryptamine (4,5-MDO-DiPT), N,N-diisopropyl-5,6-methylenedioxy-tryptamine (5,6-MDO-DiPT), N,N-dimethyl-4,5-methylenedioxy-tryptamine (4,5-MDO-DMT), N,N-dimethyl-5,6-methylenedioxy-tryptamine (5,6-MDO-DMT), N-isopropyl-N-methyl-5,6-methylenedioxy-tryptamine (5,6-MDO-M iPT), N,N-diethyl-2-methyl-tryptamine (2-Me-DET), 2,N,N-tryptaminerimethyl-tryptamine (2-Me-DMT), N-acetyl-5-methoxy-tryptamine (melatonin), N,N-diethyl-5-methoxy-tryptamine (5-MeO-DET), N,N-diisopropyl-5-methoxy-tryptamine (5-MeO-DiPT), 5-methoxy-N,N-dimethyl-tryptamine (5-MeO-DMT), N-isopropyl-4-methoxy-N-methyl-tryptamine (4-MeO-MiPT), N-isopropyl-5-methoxy-N-methyl-tryptamine (5-MeO-MiPT), 5,6-dimethoxy-N-isopropyl-N-methyl-tryptamine (5,6-MeO-MiPT), 5-methoxy-N-methyl-tryptamine (5-MeO-NMT), 5-methoxy-N,N-tetramethylene-tryptamine (5-MeO-pyr-tryptamine), 6-methoxy-1-methyl-1,2,3,4-tetrahydro-carboline (6-MeO-tryptamineHH), 5-methoxy-2,N,N-trimethyl-tryptamine (5-MeO-tryptamineMT), N,N-dimethyl-5-methylthio-tryptamine (5-MeS-DMT), N-isopropyl-N-methyl-tryptamine (MiPT), α-methyl-tryptamine (a-MT), N-ethyl-tryptamine (NET), N-methyl-tryptamine (NMT), 6-propyl-norlysergic acid (PRO-LAD), N,N-tetramethylene-tryptamine (pyr-T), Tryptamine (T), 7-methoxy-1-methyl-1,2,3,4-tetrahydro-carboline (Tetrahydroharmine), or α,N-dimethyl-5-methoxy-tryptamine (α,N,O-TMS), or a pharmaceutically acceptable salt, hydrate, solvate, prodrug, stereoisomer, or tautomer thereof, or a combination thereof. See Shulgin and Shulgin, TIHKAL: The Continuation, Transform Press (1997), which is incorporated by reference herein for the specific teachings thereof.

In another embodiment, the tryptamine comprises a compound having the structure of Formula 1,

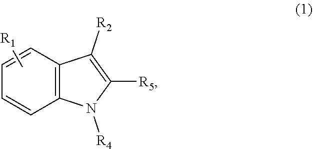

wherein
R₁ is H, OH, COOH, OCH₃, O(CH₂)ₙCH₃, (CH₂)ₙOH, (CH₂)ₙCOOH, —C(O)CH₃, (CH₂)ₙOC(O)N(R₆)₂, —(CH₂)ₙC(O)OC(O)OH, PO₄, P₂O₇, P₃O₁₀, SO₄, S₂O₇, S₃O₁₀, CHO₃, a C₁-C₆ mono- di-, or tri-carboxylic acid, a pentose sugar, a hexose sugar, or an amino acid;
R₂ is (CH₂)ₙN(R₆)₂, (CH₂)ₙO(R₆), or C₁-C₅ alkyl-N(R₇)₂;
R₄ is H, CH₃, OH, CHOCH₃, or (CH₂)ₙOH;
R₅ is H, CH₃, OH, CHOCH₃, or (CH₂)ₙCH₃;
R₆ is H, CH₃, C₁-C₄ alkyl, OH, CHOCH₃, (CH₂)ₙOH, OCH₃, O(CH₂)ₙCH₃;
R₇ is independently H, CH₃, C₁-C₄ alkyl, C₁-C₄ allyl, C₁-C₄ ethynyl, OH, COOH, (CH₂)ₙCOOH; OCH₃, O(CH₂)ₙCH₃, (CH₂)ₙOH, (CH₂)ₙNH₂; dimethyl amine, pyrrole, pyrazole, imidazole, pyridine, piperdine, pyridine, pyrimidine, indole, purine, quinoline, morpholino, pyran, or furan;
where n is 0, 1, 2, 3, or 4; and
wherein
mono-, di-, and tri-carboxylic acids is selected from acetic acid, acetylsalicylic acid, adipic acid, alginic acid, arachidic acid, ascorbic acid, aspartic acid, benzenesulfonic acid, benzoic acid, bisulfic acid, boric acid, butyric acid, camphoric acid, camphorsulfonic acid, capric acid, caproic acid, caprylic acid, carbonic acid, citric acid, cyclopentanepropionic acid, digluconic acid, dodecylsulfic acid, enanthic acid, ethanesulfonic acid, formic acid, fumaric acid, glucoheptanoic acid, gluconic acid, glutamic acid, glutaric acid, glyceric acid, glycerophosphoric acid, glycine, glycolic acid, hemisulfic acid, heptanoic acid, hexanoic acid, hippuric acid, hydrobromic acid, hydrochloric acid, hydroiodic acid, hydroxyethanesulfonic acid, lactic acid, lauric acid, maleic acid, malic acid, malonic acid, mandelic acid, margaric acid, methanesulfonic acid, mucic acid, myristic acid, naphthylanesulfonic acid, naphthylic acid, nicotinic acid, nonadecylic acid, oxalic acid, oxalic acid, palmitic acid, pelargonic, pelargonic acid, pentadecylic acid, phosphoric acid, propionic acid, saccharin, salicylic acid, sorbic acid, stearic acid, succinic acid, sulfuric acid, tartaric acid, thiocyanic acid, thioglycolic acid, thiosulfuric acid, tosylic acid, trichloroacetic acid, tridecylic acid, trifluoroacetic acid, undecylenic acid, undecylic acid, valeric acid;
triose sugars are selected from D- or L-glyceraldehyde;
tetrose sugars are selected from D- or L-erythrose or threose, and their deoxy counterparts;
pentose sugars are selected from D- or L-arabinose, lyxose, ribose, xylose, ribulose, or xylulose, and their deoxy counterparts;
hexose sugars are selected from D- or L-allose, altrose, glucose, mannose, gulose, idose, galactose, talose, psicose, fructose, sorbose, tagatose, and their deoxy counterparts;
and amino acids are selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, ornithine, citrulline, taurine, selenocysteine, pyrrolysine, aminobutyric acid, gama-aminobutryic acid, 3-aminopropanoic acid, dehydroalanine, delta-carboxyglutamic acid, N-formylmethionine.

In another embodiment, the tryptamine comprises a compound having the structure of Formula 2A or 2B,

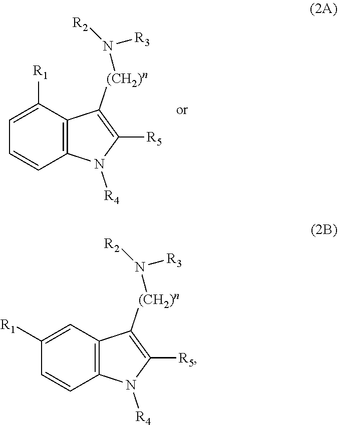

wherein:
R₁ is H, OH, COOH, OCH₃, O(CH₂)ₙCH₃, (CH₂)ₙOH, (CH₂)ₙCOOH, —C(O)CH₃, (CH₂)ₙOC(O)N(R₆)₂, —(CH₂)ₙC(O)OC(O)OH, PO₄, P₂O₇, P₃O₁₀, SO₄, S₂O₇, S₃O₁₀, CHO₃, a C₁-C₆ mono- di-, or tri-carboxylic acid, a pentose sugar, a hexose sugar, or an amino acid;
R₂ and R₃ are independently H, CH₃, C₁-C₄ alkyl, C₁-C₄ allyl, C₁-C₄ ethynyl, OH, COOH, (CH₂)ₙCOOH; OCH₃, O(CH₂)ₙCH₃, (CH₂)ₙOH, (CH₂)ₙNH₂; dimethyl amine, pyrrole, pyrazole, imidazole, pyridine, piperdine, pyridine, pyrimidine, indole, purine, quinoline, morpholino, pyran, or furan;
R₄ is H, CH₃, OH, CHOCH₃, (CH₂)ₙOH, OCH₃, O(CH₂)ₙCH₃;
R₅ is H, CH₃, OH, CHOCH₃, (CH₂)ₙOH, OCH₃, O(CH₂)ₙCH₃ or (CH₂)ₙCH₃; and
R₆ is H, CH₃, C1-C4 alkyl, OH, CHOCH₃, (CH₂)ₙOH, OCH₃, O(CH₂)ₙCH₃;
where n is 0, 1, 2, 3, or 4;
wherein
mono-, di-, and tri-carboxylic acids is selected from acetic acid, acetylsalicylic acid, adipic acid, alginic acid, arachidic acid, ascorbic acid, aspartic acid, benzenesulfonic acid, benzoic acid, bisulfic acid, boric acid, butyric acid, camphoric acid, camphorsulfonic acid, capric acid, caproic acid, caprylic acid, carbonic acid, citric acid, cyclopentanepropionic acid, digluconic acid, dodecylsulfic acid, enanthic acid, ethanesulfonic acid, formic acid, fumaric acid, glucoheptanoic acid, gluconic acid, glutamic acid, glutaric acid, glyceric acid, glycerophosphoric acid, glycine, glycolic acid, hemisulfic acid, heptanoic acid, hexanoic acid, hippuric acid, hydrobromic acid, hydrochloric acid, hydroiodic acid, hydroxyethanesulfonic acid, lactic acid, lauric acid, maleic acid, malic acid, malonic acid, mandelic acid, margaric acid, methanesulfonic acid, mucic acid, myristic acid, naphthylanesulfonic acid, naphthylic acid, nicotinic acid, nonadecylic acid, oxalic acid, oxalic acid, palmitic acid, pelargonic, pelargonic acid, pentadecylic acid, phosphoric acid, propionic acid, saccharin, salicylic acid, sorbic acid, stearic acid, succinic acid, sulfuric acid, tartaric acid, thiocyanic acid, thioglycolic acid, thiosulfuric acid, tosylic acid, trichloroacetic acid, tridecylic acid, trifluoroacetic acid, undecylenic acid, undecylic acid, valeric acid;
triose sugars are selected from D- or L-glyceraldehyde;
tetrose sugars are selected from D- or L-erythrose or threose, and their deoxy counterparts;
pentose sugars are selected from D- or L-arabinose, lyxose, ribose, xylose, ribulose, or xylulose, and their deoxy counterparts;
hexose sugars are selected from D- or L-allose, altrose, glucose, mannose, gulose, idose, galactose, talose, psicose, fructose, sorbose, tagatose, and their deoxy counterparts;
and amino acids are selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, ornithine, citrulline, taurine, selenocysteine, pyrrolysine, aminobutyric acid, gama-aminobutryic acid, 3-aminopropanoic acid, dehydroalanine, delta-carboxyglutamic acid, N-formylmethionine.

In another embodiment, the tryptamine comprises a compound having the structure of Formula 3A or 3B,

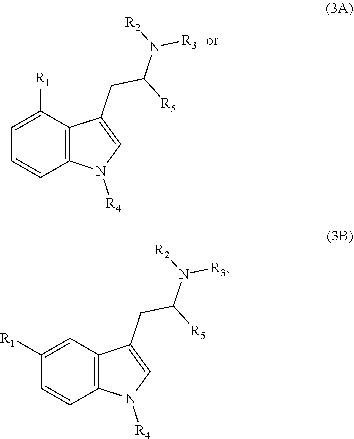

wherein:
R₁ is H, OH, COOH, OCH₃, O(CH₂)ₙCH₃, (CH₂)ₙOH, (CH₂)ₙCOOH, C(O)CH₃, (CH₂)ₙOC(O)N(R₆)₂, (CH₂)ₙC(O)OC(O)OH, PO₄, P₂O₇, P₃O₁₀, SO₄, S₂O₇, S₃O₁₀, CHO₃, a C₁-C₂₂ mono- di-, or tri-carboxylic acid, a pentose sugar, a hexose sugar, or an amino acid;
R₂ and R₃ are independently H, CH₃, (CH₂)ₙCH₃, OH, COOH, (CH₂)ₙCOOH; OCH₃, O(CH₂)ₙCH₃, (CH₂)ₙOH, (CH₂)ₙNH₂; dimethyl amine, pyrrole, pyrazole, imidazole, pyridine, piperdine, pyridine, pyrimidine, indole, purine, quinoline, morpholino, pyran, or furan;
R₄ is H, CH₃, OH, CHOCH₃, (CH₂)ₙOH, OCH₃, O(CH₂)ₙCH₃;
R₅ is H, CH₃, C₁-C₄ alkyl, OH, CHOCH₃, (CH₂)ₙOH, OCH₃, O(CH₂)ₙCH₃ or (CH₂)ₙCH₃; and
R₆ is H, CH₃, C1-C4 alkyl, OH, CHOCH₃, (CH₂)ₙOH, OCH₃, O(CH₂)ₙCH₃;
where n is 0, 1, 2, 3, or 4; and
wherein
mono-, di-, and tri-carboxylic acids is selected from acetic acid, acetylsalicylic acid, adipic acid, alginic acid, arachidic acid, ascorbic acid, aspartic acid, benzenesulfonic acid, benzoic acid, bisulfic acid, boric acid, butyric acid, camphoric acid, camphorsulfonic acid, capric acid, caproic acid, caprylic acid, carbonic acid, citric acid, cyclopentanepropionic acid, digluconic acid, dodecylsulfic acid, enanthic acid, ethanesulfonic acid, formic acid, fumaric acid, glucoheptanoic acid, gluconic acid, glutamic acid, glutaric acid, glyceric acid, glycerophosphoric acid, glycine, glycolic acid, hemisulfic acid, heptanoic acid, hexanoic acid, hippuric acid, hydrobromic acid, hydrochloric acid, hydroiodic acid, hydroxyethanesulfonic acid, lactic acid, lauric acid, maleic acid, malic acid, malonic acid, mandelic acid, margaric acid, methanesulfonic acid, mucic acid, myristic acid, naphthylanesulfonic acid, naphthylic acid, nicotinic acid, nonadecylic acid, oxalic acid, oxalic acid, palmitic acid, pelargonic, pelargonic acid, pentadecylic acid, phosphoric acid, propionic acid, saccharin, salicylic acid, sorbic acid, stearic acid, succinic acid, sulfuric acid, tartaric acid, thiocyanic acid, thioglycolic acid, thiosulfuric acid, tosylic acid, trichloroacetic acid, tridecylic acid, trifluoroacetic acid, undecylenic acid, undecylic acid, valeric acid;
triose sugars are selected from D- or L-glyceraldehyde;
tetrose sugars are selected from D- or L-erythrose or threose, and their deoxy counterparts;
pentose sugars are selected from D- or L-arabinose, lyxose, ribose, xylose, ribulose, or xylulose, and their deoxy counterparts;
hexose sugars are selected from D- or L-allose, altrose, glucose, mannose, gulose, idose, galactose, talose, psicose, fructose, sorbose, tagatose, and their deoxy counterparts;
and amino acids are selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, ornithine, citrulline, taurine, selenocysteine, pyrrolysine, aminobutyric acid, gama-aminobutryic acid, 3-aminopropanoic acid, dehydroalanine, delta-carboxyglutamic acid, N-formylmethionine.

In one embodiment, the tryptamine comprises a compound having the structure of Formula 4A or 4B,

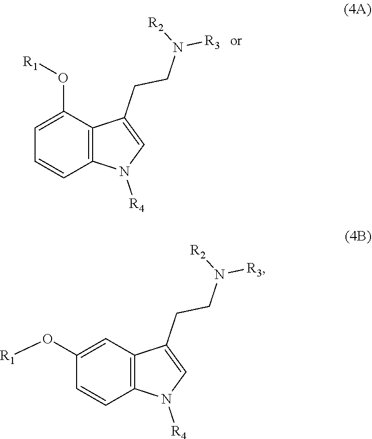

wherein:
R₁ is H, OH, COOH, OCH₃, O(CH₂)ₙCH₃, (CH₂)ₙOH, (CH₂)ₙCOOH, —C(O)CH₃, (CH₂)ₙOC(O)N(R₆)₂, —(CH₂)ₙC(O)OC(O)OH, PO₄, P₂O₇, P₃O₁₀, SO₄, S₂O₇, S₃O₁₀, CHO₃, a C₁-C₆ mono- di-, or tri-carboxylic acid, a pentose sugar, a hexose sugar, or an amino acid;
R₂ and R₃ are independently H, CH₃, OH, COOH, (CH₂)ₙCOOH; OCH₃, O(CH₂)ₙCH₃, (CH₂)ₙOH, (CH₂)ₙNH₂; dimethyl amine, pyrrole, pyrazole, imidazole, pyridine, piperdine, pyridine, pyrimidine, indole, purine, quinoline, morpholino, pyran, or furan;
R₄ is H, CH₃, OH, (CH₂)ₙOH, OCH₃, O(CH₂)ₙCH₃; and
R₆ is H, CH₃, C1-C4 alkyl, OH, CHOCH₃, (CH₂)ₙOH, OCH₃, O(CH₂)ₙCH₃;
where n is 0, 1, 2, 3, or 4; and
wherein
mono-, di-, and tri-carboxylic acids is selected from acetic acid, acetylsalicylic acid, adipic acid, alginic acid, arachidic acid, ascorbic acid, aspartic acid, benzenesulfonic acid, benzoic acid, bisulfic acid, boric acid, butyric acid, camphoric acid, camphorsulfonic acid, capric acid, caproic acid, caprylic acid, carbonic acid, citric acid, cyclopentanepropionic acid, digluconic acid, dodecylsulfic acid, enanthic acid, ethanesulfonic acid, formic acid, fumaric acid, glucoheptanoic acid, gluconic acid, glutamic acid, glutaric acid, glyceric acid, glycerophosphoric acid, glycine, glycolic acid, hemisulfic acid, heptanoic acid, hexanoic acid, hippuric acid, hydrobromic acid, hydrochloric acid, hydroiodic acid, hydroxyethanesulfonic acid, lactic acid, lauric acid, maleic acid, malic acid, malonic acid, mandelic acid, margaric acid, methanesulfonic acid, mucic acid, myristic acid, naphthylanesulfonic acid, naphthylic acid, nicotinic acid, nonadecylic acid, oxalic acid, oxalic acid, palmitic acid, pelargonic, pelargonic acid, pentadecylic acid, phosphoric acid, propionic acid, saccharin, salicylic acid, sorbic acid, stearic acid, succinic acid, sulfuric acid, tartaric acid, thiocyanic acid, thioglycolic acid, thiosulfuric acid, tosylic acid, trichloroacetic acid, tridecylic acid, trifluoroacetic acid, undecylenic acid, undecylic acid, valeric acid;
triose sugars are selected from D- or L-glyceraldehyde;
tetrose sugars are selected from D- or L-erythrose or threose, and their deoxy counterparts;
pentose sugars are selected from D- or L-arabinose, lyxose, ribose, xylose, ribulose, or xylulose, and their deoxy counterparts;
hexose sugars are selected from D- or L-allose, altrose, glucose, mannose, gulose, idose, galactose, talose, psicose, fructose, sorbose, tagatose, and their deoxy counterparts;
and amino acids are selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, ornithine, citrulline, taurine, selenocysteine, pyrrolysine, aminobutyric acid, gama-aminobutryic acid, 3-aminopropanoic acid, dehydroalanine, delta-carboxyglutamic acid, N-formylmethionine.

In another embodiment, the tryptamine comprises a compound having the structure of Formula 5A or 5B:

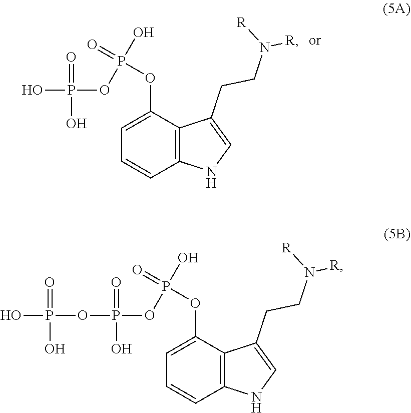

wherein each R is independently H, CH₃, CH₃CH₂, CH₃CH₂CH₂, (CH₃)₂CH, CH₂CH═CH, OCH₃, OC₁-C₄ alkyl, CH₂OH, C₁-C₃ alkyl-OH, COOH, C₁-C₃ alkyl-COOH, or a pharmaceutically acceptable salt, hydrate, solvate, or tautomer thereof, or a combination thereof.

In another embodiment, the tryptamine comprises a compound having the structure of:

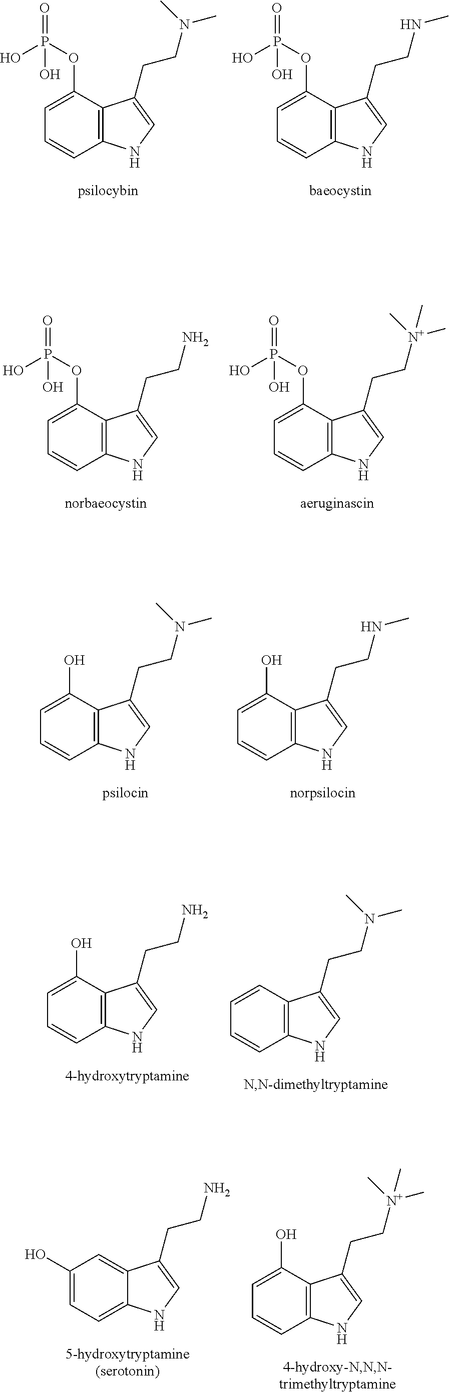

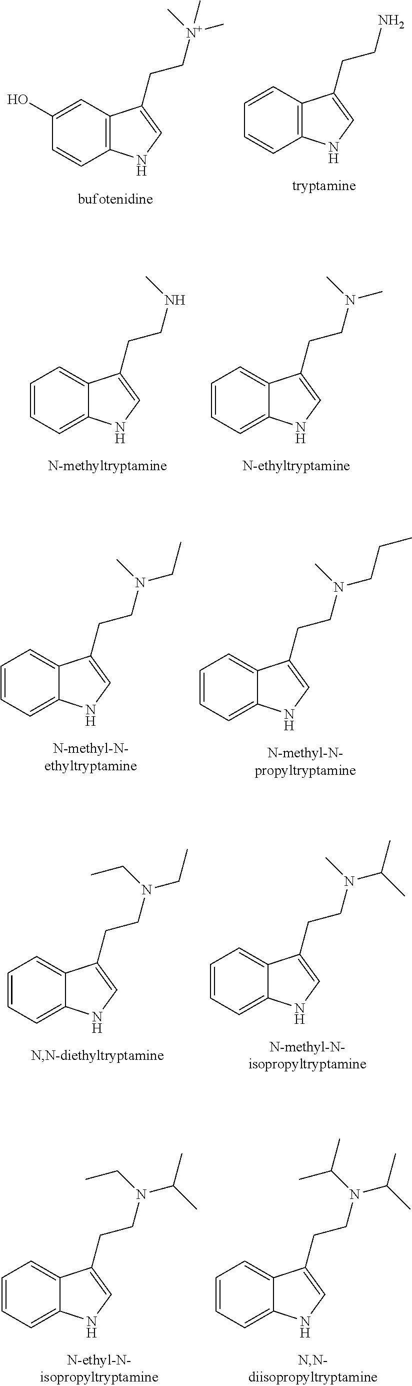

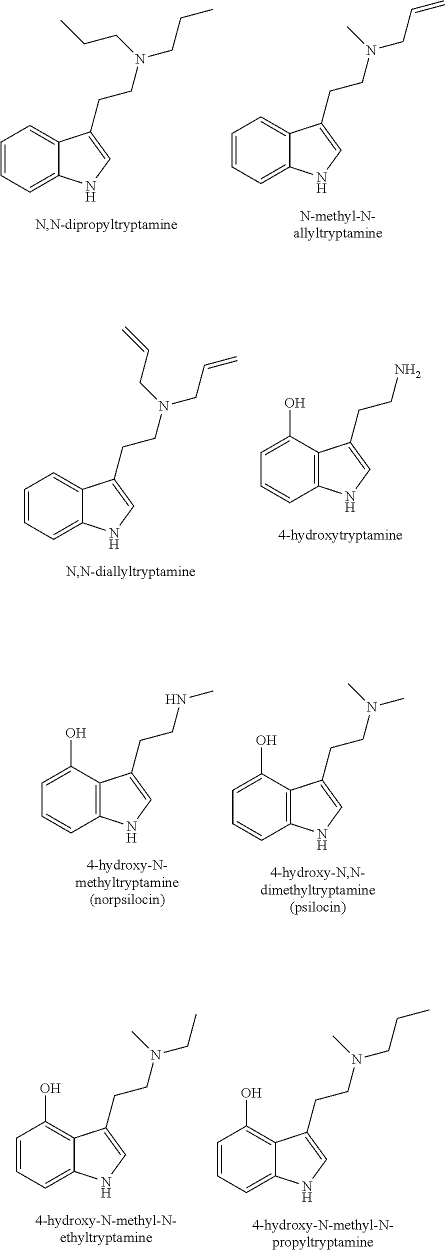

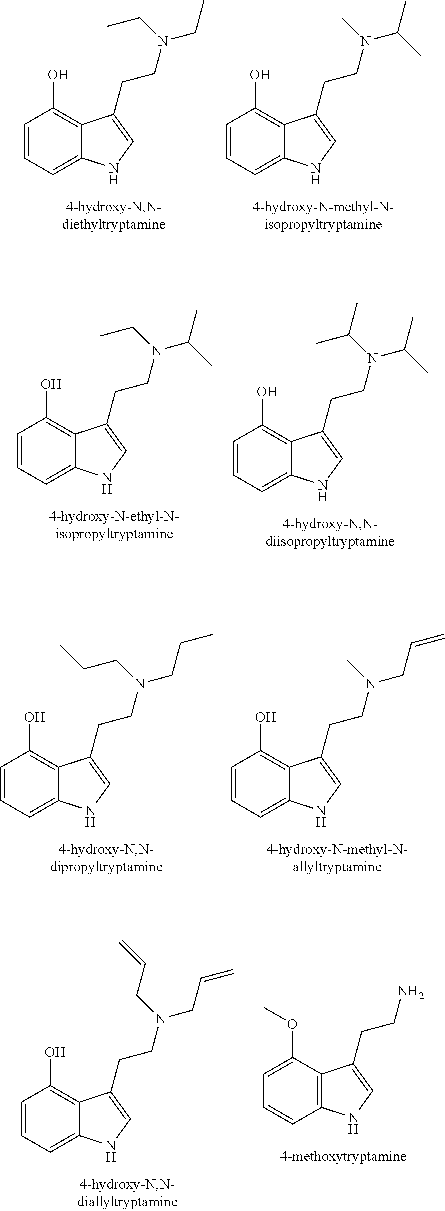

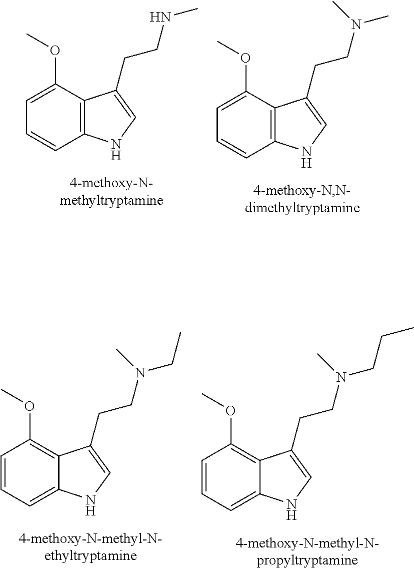

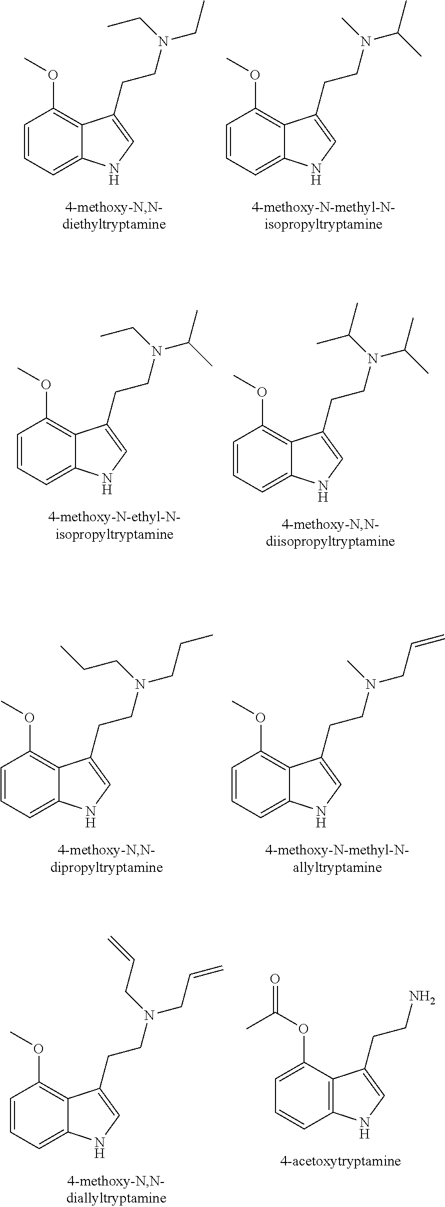

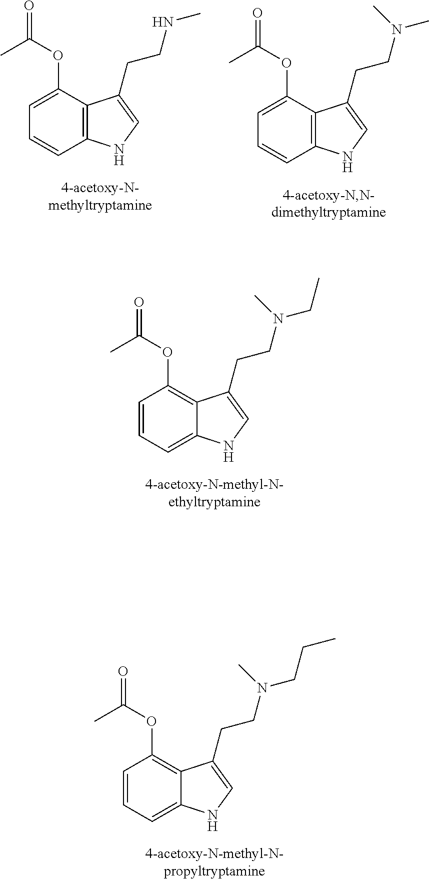 [SMILES: CNCCc1c[nH]c2cccc(OC(C)=O)c12 ; CC(=O)Oc1cccc2[nH]cc(CCN(C)C)c12 ; CCN(C)CCc1c[nH]c2cccc(OC(C)=O)c12 ; CCCN(C)CCc1c[nH]c2cccc(OC(C)=O)c12]

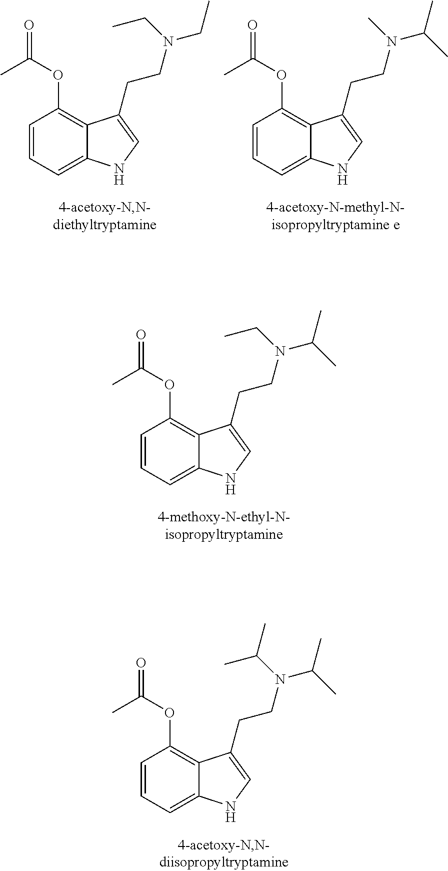 [SMILES: CCN(CC)CCc1c[nH]c2cccc(OC(C)=O)c12 ; CC(=O)Oc1cccc2[nH]cc(CCN(C)C(C)C)c12 ; CCN(CCc1c[nH]c2cccc(OC(C)=O)c12)C(C)C ; CC(=O)Oc1cccc2[nH]cc(CCN(C(C)C)C(C)C)c12]

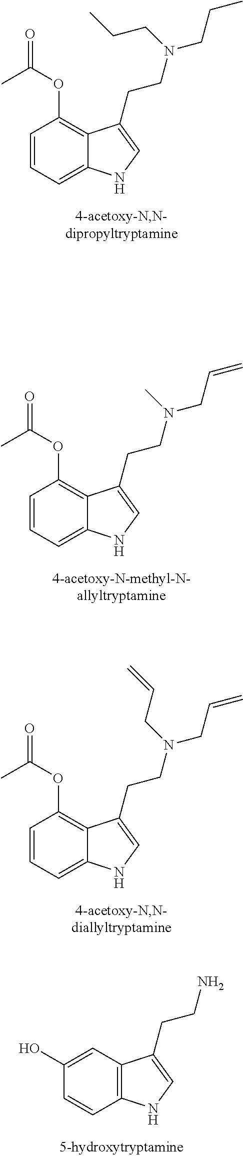

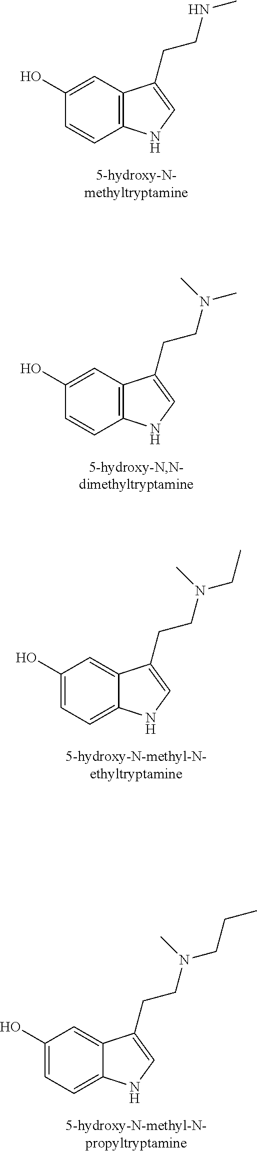 [SMILES: CNCCc1c[nH]c2ccc(O)cc12 ; CN(C)CCc1c[nH]c2ccc(O)cc12 ; CCN(C)CCc1c[nH]c2ccc(O)cc12 ; CCCN(C)CCc1c[nH]c2ccc(O)cc12]

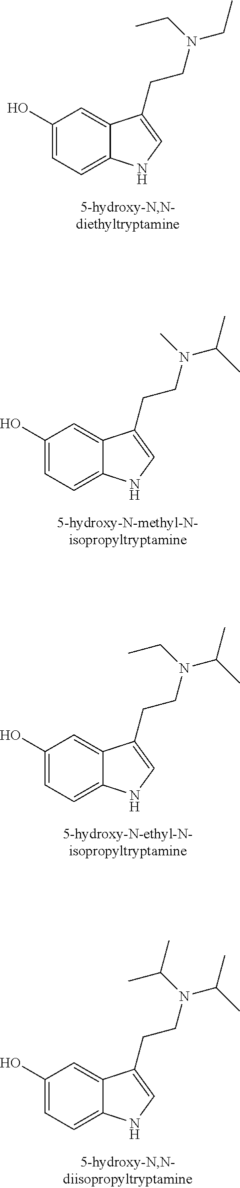 [SMILES: CCN(CC)CCc1c[nH]c2ccc(O)cc12 ; CC(C)N(C)CCc1c[nH]c2ccc(O)cc12 ; CCN(CCc1c[nH]c2ccc(O)cc12)C(C)C ; CC(C)N(CCc1c[nH]c2ccc(O)cc12)C(C)C]

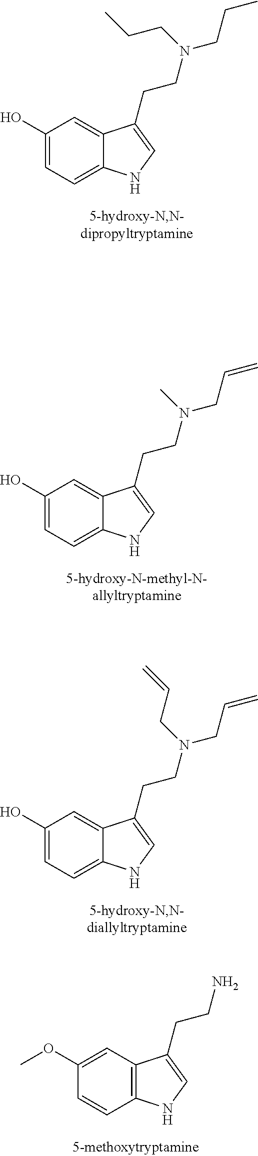

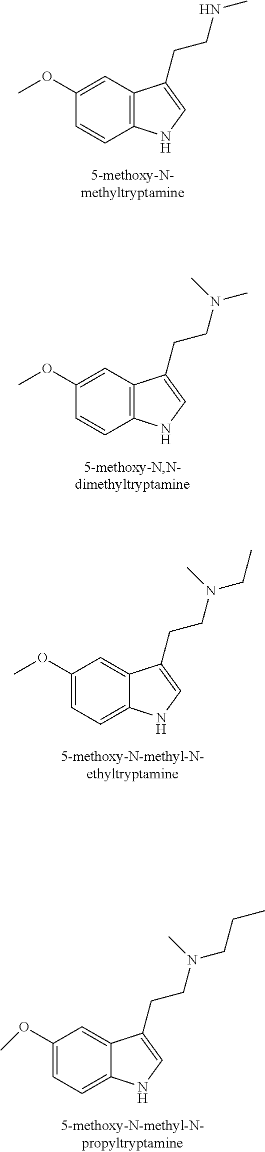

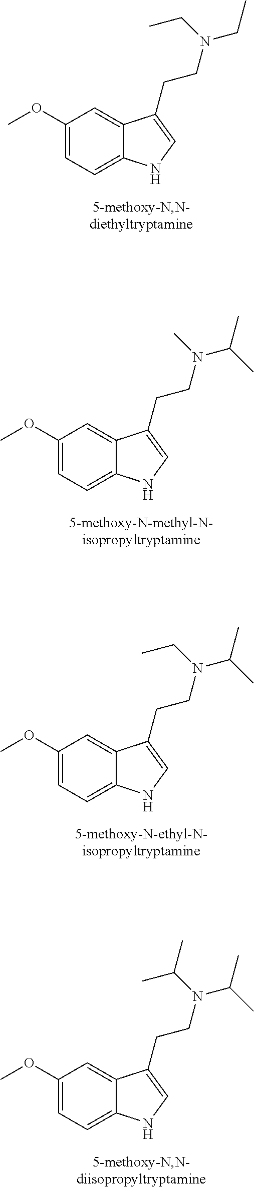

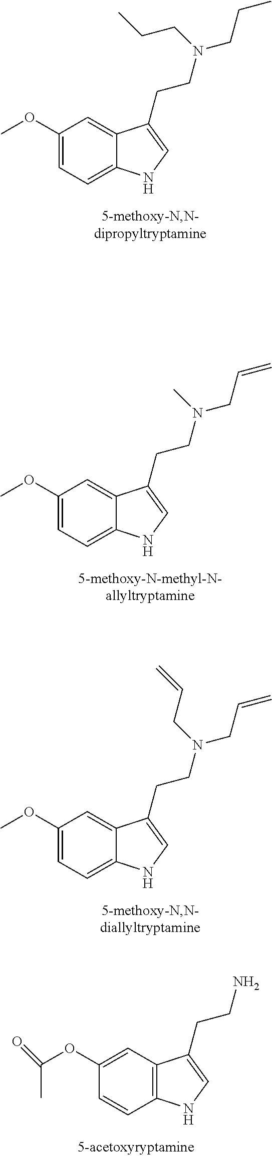

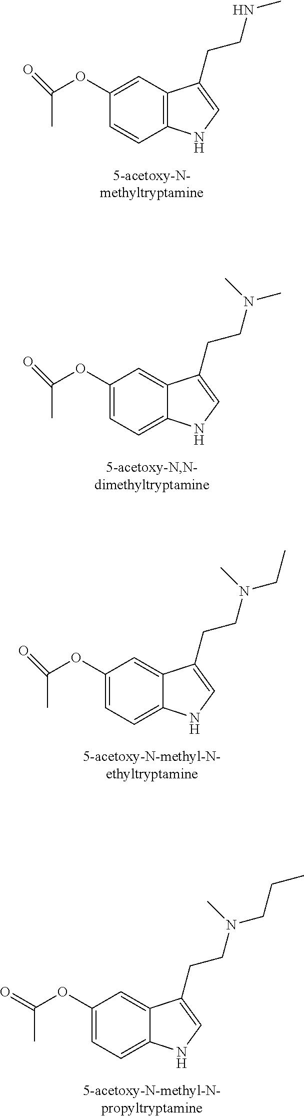 [SMILES: CNCCc1c[nH]c2ccc(OC(C)=O)cc12 ; CC(=O)Oc1ccc2[nH]cc(CCN(C)C)c2c1 ; CCN(C)CCc1c[nH]c2ccc(OC(C)=O)cc12 ; CCCN(C)CCc1c[nH]c2ccc(OC(C)=O)cc12]

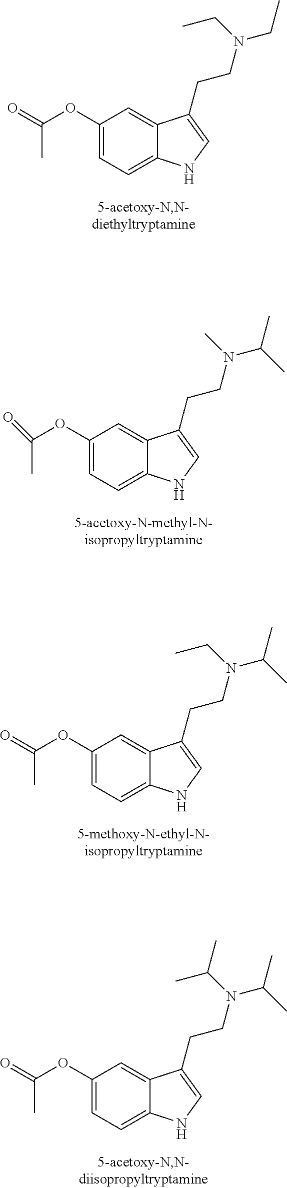 [SMILES: CCN(CC)CCc1c[nH]c2ccc(OC(C)=O)cc12 ; CC(=O)Oc1ccc2[nH]cc(CCN(C)C(C)C)c2c1 ; CCN(CCc1c[nH]c2ccc(OC(C)=O)cc12)C(C)C ; CC(=O)Oc1ccc2[nH]cc(CCN(C(C)C)C(C)C)c2c1]

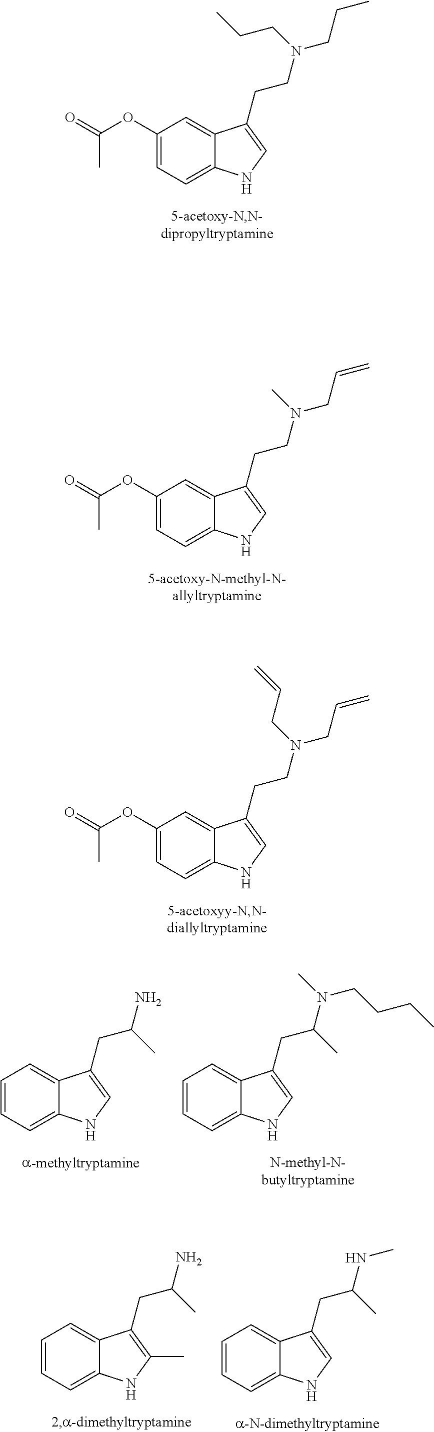

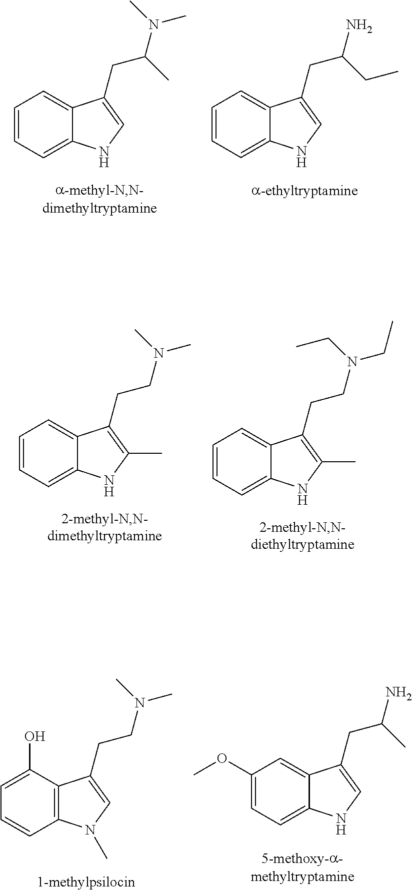 [SMILES: CC(Cc1c[nH]c2ccccc12)N(C)C ; CCC(N)Cc1c[nH]c2ccccc12 ; Cc1[nH]c2ccccc2c1CCN(C)C ; CCN(CC)CCc1c(C)[nH]c2ccccc12 ; CN(C)CCc1cn(C)c2cccc(O)c12 ; COc1ccc2[nH]cc(CC(C)N)c2c1]

In another embodiment, the composition comprises a compound having the structure of:

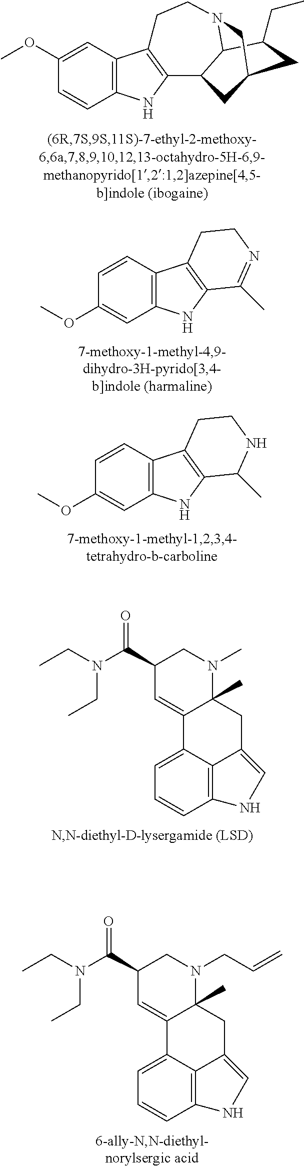

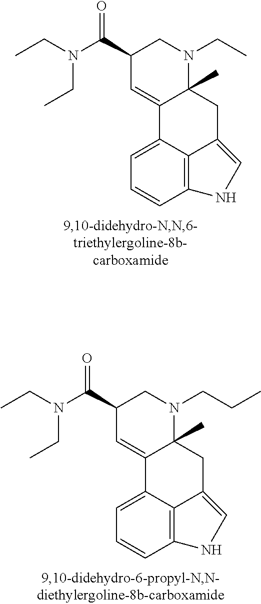

In another embodiment, the composition comprises an erinacine or hericenone in pure form, extracts or isolates from Hericium erinaceus mushroom species, or combinations thereof.

In another embodiment, the erinacine comprises Erinacine A, Erinacine B, Erinacine C, Erinacine D, Erinacine E, Erinacine F, Erinacine G, Erinacine H, Erinacine I, Erinacine J, Erinacine K, Erinacine P, Erinacine Q, Erinacine R, Erinacol, other Erinacines or a pharmaceutically acceptable salt, hydrate, solvate, prodrug, stereoisomer, or tautomer thereof, or a combination thereof.

In another embodiment, the hericenone comprises Hericenone A, Hericenone B, Hericenone C, Hericenone D, Hericenone E, Hericenone F, Hericenone G, Hericenone H, other hericenones, or a pharmaceutically acceptable salt, hydrate, solvate, prodrug, stereoisomer, or tautomer thereof, or a combination thereof.

In another embodiment, the hericenone comprises Hericenone A, Hericenone B, Hericenone C, Hericenone D, Hericenone E, Hericenone F, Hericenone G, Hericenone H, Erinacine I, Erinacine J, Erinacine K, Erinacine P, Erinacine Q, Erinacine R, Erinacol, other hericenones, or a pharmaceutically acceptable salt, hydrate, solvate, prodrug, stereoisomer, or tautomer thereof, or a combination thereof.

In another embodiment, the erinacine comprises a compound having the structure of:

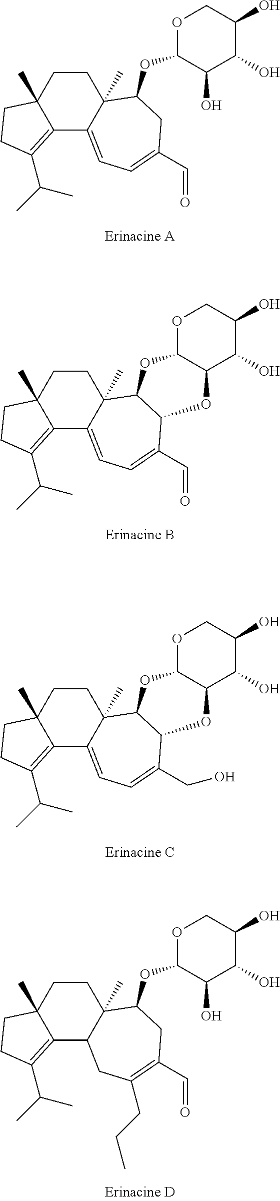 [SMILES: CC(C)C1=C2C3=CC=C(C=O)C[C@H](O[C@@H]4OC[C@@H](O)[C@H](O)[C@H]4O)[C@]3(C)CC[C@@]2(C)CC1 ; CC(C)C1=C2C3=CC=C(C=O)[C@H]4O[C@H]5[C@@H](OC[C@@H](O)[C@@H]5O)O[C@@H]4[C@]3(C)CC[C@@]2(C)CC1 ; CC(C)C1=C2C3=CC=C(CO)[C@H]4O[C@H]5[C@@H](OC[C@@H](O)[C@@H]5O)O[C@@H]4[C@]3(C)CC[C@@]2(C)CC1 ; CCCC1=C(C=O)C[C@H](O[C@@H]2OC[C@@H](O)[C@H](O)[C@H]2O)[C@]2(C)CC[C@@]3(C)CCC(C(C)C)=C3C2C1]

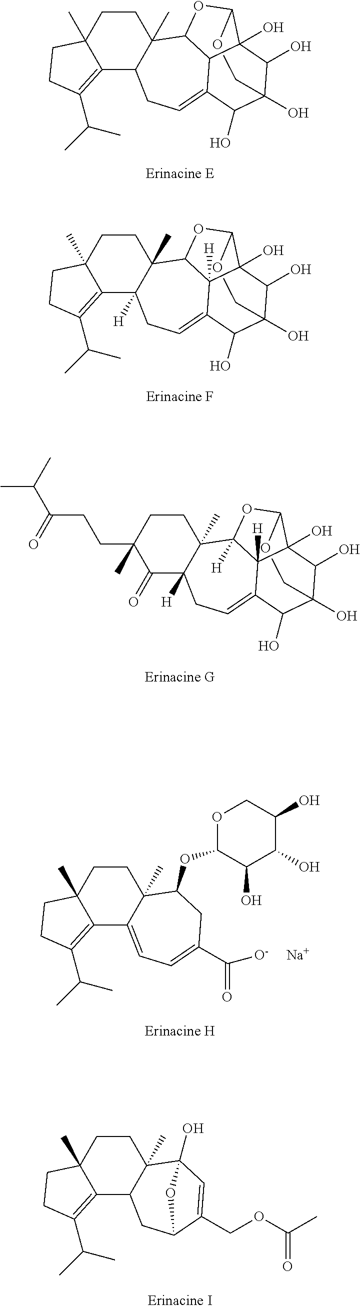

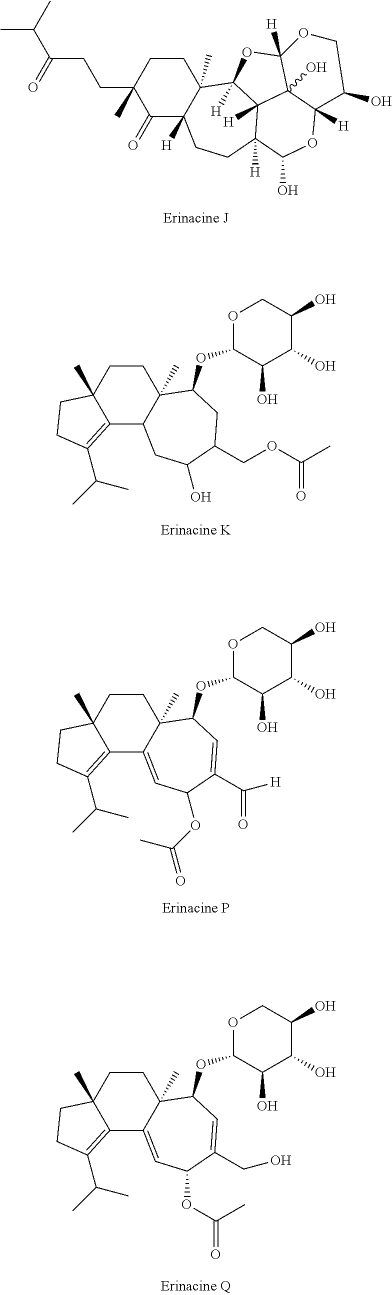

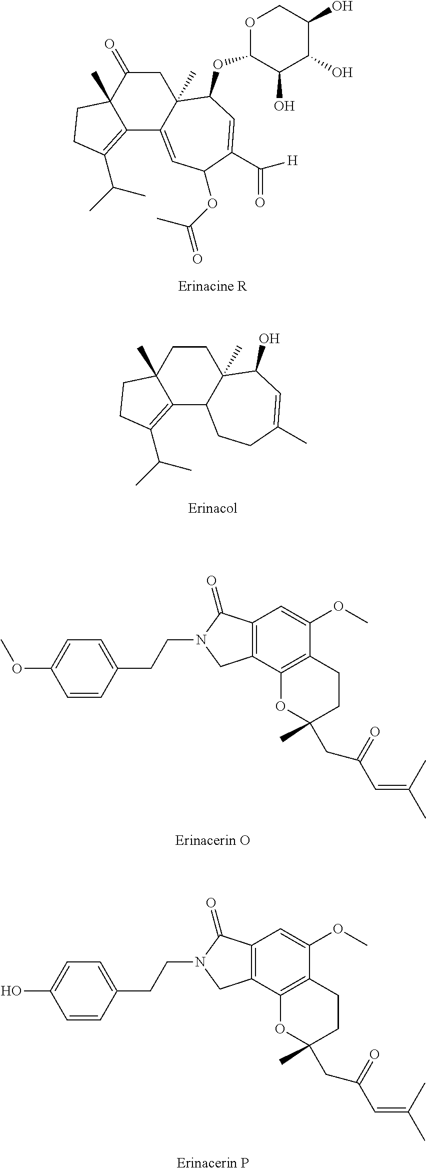 [SMILES: CC(=O)OC1C=C2C3=C(C(C)C)CC[C@]3(C)C(=O)C[C@@]2(C)[C@@H](O[C@@H]2OC[C@@H](O)[C@H](O)[C@H]2O)C=C1C=O ; CC1=C[C@H](O)[C@]2(C)CC[C@@]3(C)CCC(C(C)C)=C3C2CC1 ; COc1ccc(CCN2Cc3c(cc(OC)c4c3O[C@@](C)(CC(=O)C=C(C)C)CC4)C2=O)cc1 ; COc1cc2c(c3c1CC[C@](C)(CC(=O)C=C(C)C)O3)CN(CCc1ccc(O)cc1)C2=O]

In another embodiment, the hericenone comprises Hericenone A, Hericenone B, Hericenone C, Hericenone D, Hericenone E, Hericenone F, Hericenone G, Hericenone H, other hericenones, or a pharmaceutically acceptable salt, hydrate, solvate, prodrug, stereoisomer, or tautomer thereof, or a combination thereof.

In another embodiment, the hericenone comprises a compound having the structure of:

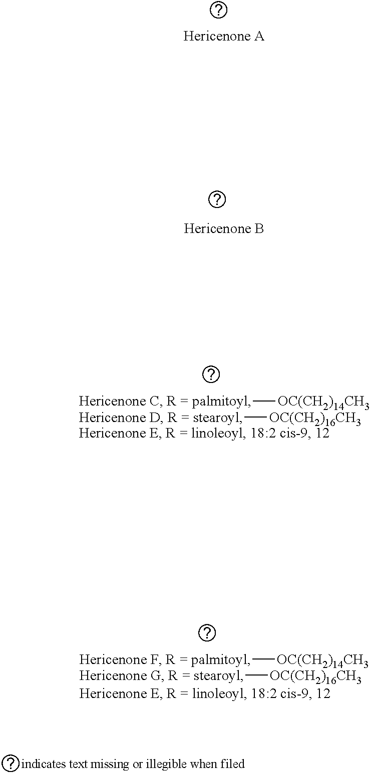

In another embodiment, the active compound is a compound isolated and identified in an extract from Hericium erinaceus.

In another embodiment, the compositions as described herein may comprise a cannabinoid comprising Δ8-tetrahydrocannabinol (THC), Δ9-tetrahydrocannabinol, tetrahydrocannabinolic acid (THCA), cannabidiol (CBD), cannabidiolic acid (CBDA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabielsoin (CBE), cannabicitran (CBT), other cannabinoids, or a pharmaceutically acceptable salt, hydrate, solvate, prodrug, stereoisomer, or tautomer thereof, or a combination thereof.

In another embodiment, the cannabinoid comprises a compound having the structure of:

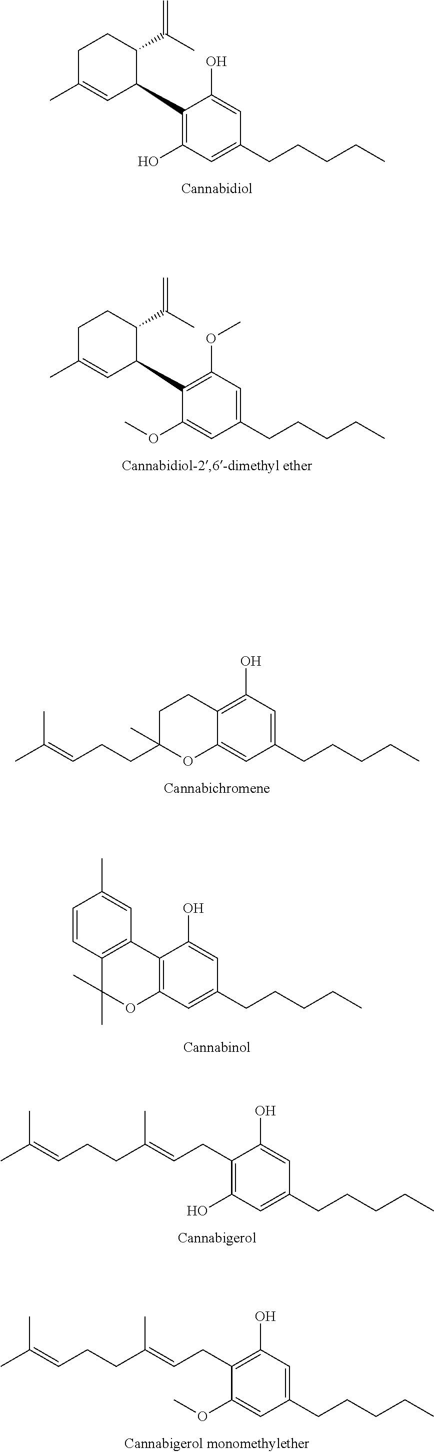

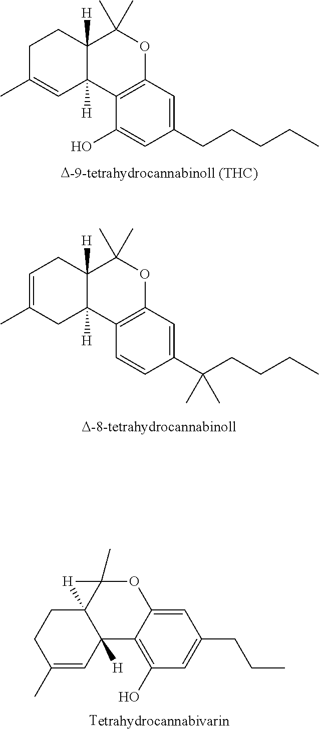

In another embodiment, the compositions described herein may comprise one or more natural products such as aliphatic natural products, alkaloids, amino acids, anthranilic acid alkaloids, apiole, (+)-aromanderndrene, asarone, aurones, benzofuranoids, benzofurans, benzophenones, benzopyranoids, benzopyrans, benztropolones, cis-α-bergamotene, trans-α-bergamotene, α-bisabolol, borneol, γ-cadinene, caffeic acid, camphor, carbohydrates, carotenoids, 3-carene, β-carbolines, trans-β-caryophyllene, catechins, chalcones, chavicol, chavicols, chromones, cineol, cinnamic acid, cinnamic aldehydes, cinnamic monolignols, conferyl alcohol, coniferyl alcohol, cordysinin, coumarins, coumaric acid, coumaryl alcohol, cutin, depsides, depsidones, dillapiole, diterpenes, diterpenoids, γ-elemene, elemicin, eleutherosides, esterterpenoids, estragole, eudesman-3,7(11)-diene, β-eudesmol, γ-eudesmol, eugenol, trans-β-farnesene, ferulic acid, haramane, harmine, norharmine, harmol, α-humuline, β-fenchol, 5-hydroxyferulic acid, flavonoids, glycopeptides, hydroxycinnamic acids, hydroxylated fatty acids, imidazole alkaloids, isoflavonoids, isoquinoline alkaloids, β-lactams, lignans, limonoids, R-limonene, (−)-linalool, lipids, lysine alkaloids, meroterpenoids, methyl eugenol, miscellaneous terpenoids, monoterpenoid indole alkaloids, monoterpenoids, myrcene, myristicin, nerolidol, nicotinic acid alkaloids, cis-ocimene, 1-octanol, ornithine alkaloids, otenoids, oxazole alkaloids, oxygen heterocycles, peptides, phellanderene, phenolics, phenylalanine alkaloids, phenylpropanoids, phenylpropanoids, phenylpropenes, perlolyrine, pinene, polycyclic aromatic natural products, polyketide alkaloids, polyketides, polypyrroles, ptteridines, purines, putrescine alkaloids, pyrazine alkaloids, pyrimidines, pyrrole alkaloids, quassinoids, quinonemethides, quinones, quinoxaline alkaloids, resveratrol, trans-resveratrol, cis-sabinene hydrate, safrole, γ-selinene, semiochemicals, septide alkaloids, sesquiterpenes, sesquiterpenoids, simple aromatic natural products, sinapic acid, sinapyl alcohols, spermidine alkaloids, spermine alkaloids, sporopollenin, steroidal alkaloids, steroids, sterols, stilbenes, stilbenoids, suberin, tannins, terpenoid alkaloids, terpenoids, γ-terpinene, α-terpineol, terpinolene, tetraterpenoids, thiazole alkaloids, triterpenes, triterpenoids, tryptophan alkaloids, tyrosine alkaloids, umbelliferone, xanthones, or a pharmaceutically acceptable salt, hydrate, solvate, prodrug, stereoisomer, or tautomer thereof, or a combination thereof.

In another embodiment, the compositions described herein may comprise a phenethylamine or an amphetamine compound selected from: α-ethyl-3,4,5-trimethoxy-phenethylamine (AEM), 4-allyloxy-3,5-dimethoxy-phenethylamine (AL), 4-methylthio-2,5-dimethoxy-amphetamine (ALEPH), 4-ethylthio-2,5-dimethoxy-amphetamine (ALEPH-2), 4-isopropylthio-2,5-dimethoxy-amphetamine (ALEPH-4), 4-phenylthio-2,5-dimethoxy-amphetamine (ALEPH-6), 4-propylthio-2,5-dimethoxy-amphetamine (ALEPH-7), 2,5-dimethoxy-α-ethyl-4-methyl-phenethylamine (ARIADNE), 3,4-diethoxy-5-methoxy-phenethylamine (ASB), 4-butoxy-3,5-dimethoxy-phenethylamine (B), 2,5-dimethoxy-4,N-dimethyl-amphetamine (BEATRICE), 2,5-bismethylthio-4-methyl-amphetamine (BIS-TOM), 4-bromo-2,5,3-trimethoxy-phenethylamine (BOB), 2,5,3-trimethoxy-4-methyl-phenethylamine (BOD), 3-methoxy-3,4-methylenedioxy-phenethylamine (BOH), 2,5-dimethoxy-3-hydroxy-4-methyl-phenethylamine (BOH D), 3,4,5,3-tetramethoxy-phenethylamine (BOM), 4-bromo-3,5-dimethoxy-amphetamine (4-Br-3,5-DMA), 2-bromo-4,5-methylenedioxy-amphetamine (2-Br-4,5-MDA), 4-bromo-2,5-dimethoxy-phenethylamine (2C-B), 4-benzyloxy-3,5-dimethoxy-amphetamine (3C-BZ), 4-chloro-2,5-dimethoxy-phenethylamine (2C-C), 4-methyl-2,5-dimethoxy-phenethylamine (2C-D), 4-ethyl-2,5-dimethoxy-phenethylamine (2C-E), 4-ethoxy-3,5-dimethoxy-amphetamine (3C-E), 4-fluoro-2,5-dimethoxy-phenethylamine (2C-F), 3,4-dimethyl-2,5-dimethoxy-phenethylamine (2C-G), 3,4-trimethylene-2,5-dimethoxy-phenethylamine (2C-G-3), 3,4-tetramethylene-2,5-dimethoxy-phenethylamine (2C-G-4), 3,4-norbornyl-2,5-dimethoxy-phenethylamine (2C-G-5), 1,4-dimethoxynaphthyl-2-ethylamine (2C-G-N), 2,5-dimethoxy-phenethylamine (2C-H), 4-iodo-2,5-dimethoxy-phenethylamine (2C-1), 4-nitro-2,5-dimethoxy-phenethylamine (2C-N), 4-isopropoxy-30 2,5-dimethoxy-phenethylamine (2C-O-4), 4-propyl-2,5-dimethoxy-phenethylamine (2C-P), 4-cyclopropylmethoxy-3,5-dimethoxy-phenethylamine (CPM), 4-methylseleno-2,5-dimethoxy-phenethylamine (2C-SE), 4-methylthio-2,5-dimethoxy-phenethylamine (2C-T), 4-ethylthio-2,5-dimethoxy-phenethylamine (2C-T-2), 4-isopropylthio-2,5-dimethoxy-phenethylamine (2C-T-4), 4-isopropylthio-2,6-dimethoxy-phenethylamine (psi-2C-T-4), 4-propylthio-2,5-dimethoxy-phenethylamine (2C-T-7), 4-cyclopropylmethylthio-2,5-dimethoxy-phenethylamine (2C-T-8), 4-(t)-butylthio-2,5-dimethoxy-phenethylamine (2C-T-9), 4-(2-methoxyethylthio)-2,5-dimethoxy-phenethylamine (2C-T-13), 4-cyclopropylthio-2,5-dimethoxy-phenethylamine (2C-T-15), 4-(s)-butylthio-2,5-dimethoxy-phenethylamine (2C-T-17), 4-(2-fluoroethylthio)-2,5-dimethoxy-phenethylamine (2C-T-21), 4-trideuteromethyl-3,5-dimethoxy-phenethylamine (4-D), β,β-dideutero-3,4,5-trimethoxy-phenethylamine (β-D), 4-methyl-3,5-dimethoxy-phenethylamine (DESOXY), 2,4-dimethoxy-amphetamine (2,4-DMA), 2,5-dimethoxy-amphetamine (2,5-DMA), 3,4-d methoxy-amphetamine (3,4-DMA), 2-(2,5-dimethoxy-4-methylphenyl)-cyclopropylamine (DMCPA), 3,4-dimethoxy-β-hydroxy-phenethylamine (DME), 2,5-dimethoxy-3,4-methylenedioxy-amphetamine (DMMDA), 2,3-dimethoxy-4,5-methylenedioxy-amphetamine (DMMDA-2), 3,4-dimethoxy-phenethylamine (DMPEA), 4-amyl-2,5-dimethoxy-amphetamine (DOAM), 4-bromo-2,5-dimethoxy-amphetamine (DOB), 4-butyl-2,5-dimethoxy-amphetamine (DOBU), 4-chloro-2,5-dimethoxy-amphetamine (DOC), 4-(2-fluoroethyl)-2,5-dimethoxy-amphetamine (DOEF), 4-ethyl-2,5-dimethoxy-amphetamine (DOET), 4-iodo-2,5-dimethoxy-amphetamine (DOI), 4-methyl-2,5-dimethoxy-amphetamine (DOM (STP)), 4-methyl-2,6-dimethoxy-amphetamine (psi-DOM), 4-nitro-2,5-dimethoxy-amphetamine (DON), 4-propyl-2,5-dimethoxy-amphetamine (DOPR), 4-ethoxy-3,5-dimethoxy-phenethylamine (E), 2,4,5-triethoxy-amphetamine (EEE), 2,4-diethoxy-5-methoxy-amphetamine (EEM), 2,5-diethoxy-4-methoxy-amphetamine (EME), 2-ethoxy-4,5-dimethoxy-amphetamine (EMM), N,α-diethyl-3,4-methylenedioxy-phenethylamine (ETHYL-J), N-ethyl-α-propyl-3,4-methylenedioxy-phenethylamine (ETHYL-K), benzofuran-2-methyl-5-methoxy-6-(2-aminopropane) (F-2), benzofuran-2,2-dimethyl-5-methoxy-6-(2-aminopropane) (F-22), N-hydroxy-N-methyl-3,4-methylenedioxy-amphetamine (FLEA), 3,4-trimethylene-2,5-dimethoxy-amphetamine (G-3), 3,4-tetramethylene-2,5-dimethoxy-amphetamine (G-4), 3,4-norbornyl-2,5-dimethoxy-amphetamine (G-5), 3,4-dimethyl-2,5-dimethoxy-amphetamine (GANESHA), 1,4-dimethoxynaphthyl-2-isopropylamine (G-N), 2,5-dimethoxy-N-hydroxy-4-ethylthio-phenethylamine (HOT-2), 2,5-dimethoxy-N-hydroxy-4-(n)-propylthio-phenethylamine (HOT-7), 2,5-dimethoxy-N-hydroxy-4-(s)-butylthio-phenethylamine (HOT-17), 2,5-dimethoxy-N,N-dimethyl-4-iodo-amphetamine (IDNNA), 2,3,4-trimethoxy-phenethylamine (IM), 3,5-dimethoxy-4-isopropoxy-phenethylamine (IP), 5-ethoxy-2-methoxy-4-methyl-amphetamine (IRIS), α-ethyl-3,4-methylenedioxy-phenethylamine (J), 3-methoxy-4,5-methylenedioxy-phenethylamine (LOPHOPHINE), 3,4,5-trimethoxy-phenethylam ine (M), 4-methoxy-amphetamine (4-MA), 2,N-dimethyl-4,5-methylenedioxy-amphetamine (MADAM-6), 3,5-dimethoxy-4-methallyloxy-phenethylamine (MAL), 3,4-methylenedioxy-amphetamine (MDA), N-allyl-3,4-methylenedioxy-amphetamine (MDAL), N-butyl-3,4-methylenedioxy-amphetamine (MDBU), N-benzyl-3,4-methylenedioxy-amphetamine (MDBZ), N-Cyclopropylmethyl-3,4-methylenedioxy-amphetamine (MDCPM), N,N-dimethyl-3,4-methylenedioxy-amphetamine (MDDM), N-ethyl-3,4-methylenedioxy-amphetamine (MDE), N-(2-hydroxyethyl)-3,4-methylenedioxy-amphetamine (MDHOET), N-isopropyl-3,4-methylenedioxy-amphetamine (MDIP), N-methyl-3,4-methylenedioxy-amphetamine (MDMA), N-methyl-3,4-ethylenedioxy-amphetamine (MDMC), N-methoxy-3,4-methylenedioxy-amphetamine (MDMEO), N-(2-methoxyethyl)-3,4-methylenedioxy-amphetamine (MDMEOET), α,α,N-trimethyl-3,4-methylenedioxy-phenethylamine (MDMP), N-hydroxy-3,4-methylenedioxy-amphetamine (MDOH), 3,4-methylenedioxy-phenethylamine (MDPEA), α,α-dimethyl-3,4-methylenedioxy-phenethylamine (MDPH), N-propargyl-3,4-methylenedioxy-amphetamine (MDPL), N-propyl-3,4-methylenedioxy-amphetamine (MDPR), 3,4-dimethoxy-5-ethoxy-phenethylamine (ME), 3-methoxy-4,5-ethylenedioxy-amphetamine (MEDA), 2-methoxy-4,5-diethoxy-amphetamine (MEE), 2,5-dimethoxy-4-ethoxy-amphetamine (MEM), 3-methoxy-4-ethoxy-phenethylamine (MEPEA), 5-bromo-2,4-dimethoxy-amphetamine (META-DOB), 5-methylthio-2,4-dimethoxy-amphetamine (META-DOT), N-methyl-2,5-dimethoxy-amphetamine (METHYL-DMA), 4-bromo-2,5-dimethoxy-N-methyl-amphetamine (METHYL-DOB), N-methyl-α-ethyl-3,4-methylenedioxy-phenethylamine (METHYL-J), N-methyl-α-propyl-3,4-methylenedioxy-phenethylamine (METHYL-K), N-methyl-4-methoxy-amphetamine (METHYL-MA), N-methyl-2-methoxy-4,5-methylenedioxy-amphetamine (METHYL-MMDA-2), 3-methoxy-4,5-methylenedioxy-amphetamine (MMDA), 2-methoxy-4,5-methylenedioxy-amphetamine (MMDA-2), 2-methoxy-3,4-methylenedioxy-amphetamine (MMDA-3a), 4-methoxy-2,3-methylenedioxy-amphetamine (MMDA-3b), 2,4-dimethoxy-5-ethoxy-amphetamine (MME), 3,4-dimethoxy-5-propoxy-phenethylamine (MP), 2,5-dimethoxy-4-propoxy-amphetamine (MPM), 2-methylthio-4,5-dimethoxy-amphetamine (ORTHO-DOT), 3,5-dimethoxy-4-propoxy-phenethylamine (P), 3,5-dimethoxy-4-phenethyloxy-phenethylamine (PE), phenethylamine (PEA), 4-propynyloxy-3,5-dimethoxy-phenethylamine (PROPYNYL), 3,5-diethoxy-4-methoxy-phenethylamine (SB), 2,3,4,5-Tetramethoxy-amphetamine (TA), 4-ethoxy-3-ethylthio-5-methoxy-phenethylamine (3-TASB), 3-ethoxy-4-ethylthio-5-methoxy-phenethylamine (4-TASB), 3,4-diethoxy-5-methylthio-phenethylamine (5-TASB), 4-thiobutoxy-3,5-dimethoxy-phenethylamine (TB), 4-ethoxy-5-methoxy-3-methylthio-phenethylamine (3-TE), 3,5-dimethoxy-4-ethylthio-phenethylamine (4-TE), 2-methylthio-3,4-dimethoxy-phenethylamine (2-TIM), 3-methylthio-2,4-dimethoxy-phenethylamine (3-TIM), 4-methylthio-2,3-dimethoxy-phenethylamine (4-TIM), 3-methylthio-4,5-dimethoxy-phenethylamine (3-TM), 4-methylthio-3,5-dimethoxy-phenethylamine (4-TM), 3,4,5-trimethoxy-amphetamine (TMA), 2,4,5-trimethoxy-amphetamine (TMA-2), 2,3,4-trimethoxy-amphetamine (TMA-3), 2,3,5-trimethoxy-amphetamine (TMA-4), 2,3,6-trimethoxy-amphetamine (TMA-5), 2,4,6-trimethoxy-amphetamine (TMA-6), 4,5-dimethoxy-3-ethylthio-phenethylamine (3-TME), 3-ethoxy-5-methoxy-4-methylthio-phenethylamine (4-TME), 3-ethoxy-4-methoxy-5-methylthio-phenethylamine (5-TME), 2-methylthio-3,4-methylenedioxy-amphetamine (2T-MMDA-3a), 4,5-thiomethyleneoxy-2-methoxy-amphetamine (4T-MMDA-2), 2,4,5-trimethoxy-phenethylamine (TMPEA), 4-ethyl-5-methoxy-2-methylthio-amphetamine (2-TOET), 4-ethyl-2-methoxy-5-methylthio-amphetamine (5-TOET), 5-methoxy-4-methyl-2-methylthio-amphetamine (2-TOM), 2-methoxy-4-methyl-5-methylthio-amphetamine (5-TOM), 2-methoxy-4-methyl-5-methylsulfinyl-amphetamine (TOMSO), 4-propylthio-3,5-dimethoxy-phenethylamine (TP), 3,4,5-triethoxy-phenethylamine (TRIS), 3-ethoxy-5-ethylthio-4-methoxy-phenethylamine (3-TSB), 3,5-diethoxy-4-methylthio-phenethylamine (4-TSB), 4,5-diethoxy-3-ethylthio-phenethylamine (3-T-TRIS), 3,5-diethoxy-4-ethylthio-phenethylamine (4-T-TRIS), (R)-2,5-dimethoxy-4-iodoamphetamine, or a pharmaceutically acceptable salt, hydrate, solvate, prodrug, stereoisomer, or tautomer thereof, or a combination thereof. See Shulgin and Shulgin, PIHKAL: A Chemical Love Story, Transform Press (1994), which is incorporated by reference herein for the specific teachings thereof. In some embodiments, the amphetamine may be (R)-2,5-dimethoxy-4-iodoamphetamine or a pharmaceutically acceptable salt, hydrate, solvate, prodrug, stereoisomer, or tautomer thereof. (R)-2,5-dimethoxy-4-iodoamphetamine (i.e., 2C-H) is structurally similar to the popular psychedelic drug 2C-B (which is similar to ecstasy and MDMA), but it does not itself have any psychoactive effects. It was found to activate the 5-HT_{2A} receptor and prevent and reverse inflammation in the lungs. Flanagan and Nichols, Int. Review of Psychiatry, 30(4): 363-375 (2018). In another embodiment, the amphetamine comprises (R)-2,5-dimethoxy-4-iodoamphetamine, having the structure:

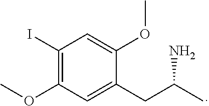

### Pharmaceutically Acceptable Salts

Pharmaceutically acceptable salts of the compounds described herein are also contemplated for the uses described herein. As used herein, the terms “salt” or “salts” refer to an acid addition or base addition salt of a compound described herein. “Salts” include in particular “pharmaceutical acceptable salts.” The term “pharmaceutically acceptable salts” refers to salts that retain the biological effectiveness and properties of the compounds disclosed herein and, which typically are not biologically or otherwise undesirable. In many cases, the compounds disclosed herein can form acid and/or base salts by virtue of the presence of amino and/or carboxyl groups or groups similar thereto.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium, and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine, and tromethamine.

Another embodiment is a tryptamine, erinacine, or hericenone as an acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate, trifenatate, trifluoroacetate, or xinafoate salt form.

### Pharmaceutical Compositions

Another embodiment is a pharmaceutical composition comprising one or more compounds described herein or a pharmaceutically acceptable salt, hydrate, solvate, prodrug, stereoisomer, or tautomer thereof, and one or more pharmaceutically acceptable carrier(s). The term “pharmaceutically acceptable carrier” refers to a pharmaceutically acceptable material, composition, or vehicle, such as a liquid or solid filler, diluent, excipient, solvent, or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions of the disclosure are administered orally, intraperitoneally, or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tween®, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions described herein may be orally administered in any orally acceptable dosage form including, but not limited to, capsules, tablets, aqueous suspensions, or solutions. In the case of tablets for oral use, carriers commonly used include lactose and com starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring, or coloring agents may also be added.

Alternatively, the pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax, and polyethylene glycols.

The pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract can be administered using a rectal suppository formulation (see above) or a suitable enema formulation. Topically transdermal patches may also be used.

For topical applications, the pharmaceutically acceptable compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax, and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol, and water.

The pharmaceutically acceptable compositions of this disclosure may also be administered by nasal aerosol or inhalation. Such compositions are prepared according to techniques known in the art of pharmaceutical formulation and may be prepared as solutions in saline, employing benzyl alcohol or other suitable preservatives, absorption promoters to enhance bioavailability, fluorocarbons, or other conventional solubilizing or dispersing agents. The amount of the compounds of the present disclosure that may be combined with the carrier materials to produce a composition in a single dosage form will vary depending upon the host treated and the mode of administration. Preferably, the compositions should be formulated so that a dosage of between 0.01-100 mg/kg body weight/day can be administered to a patient receiving these compositions. In one embodiment, the composition is described as shown below in Table 3. Compositions may contain one or more species, or combinations of any of the species, listed below in Table 3.

TABLE 3
Exemplary Compositions

| Component | Example | Dosage |
|---|---|---|
| Tryptamines, tryptamine | Psilocybin, baeocystin, norbaeocystin, | 1 ng to 10 mg |
| derivatives, esters, or | psilocin, norpsilocin, 4-hydroxytryptamine, | |
| salts thereof, or extracts | N,N-dimethyltryptamine, N-methyltryptamine, | |
| from fungi or plants. In | inter alia. In addition or alternatively, 3,4,5- | |
| addition to or alternatively, | trimethoxyphenethylamine (Mescaline), 2,4- | |
| phenethylamines, | dimethoxy-amphetamine (2,4-DMA), 3,4- | |
| amphetamines; | dimethoxy-amphetamine (3,4-DMA), 3,4- | |
| derivatives thereof, | methylenedioxy-amphetamine (MDA), 3- | |
| extracts from fungi or | methoxy-4,5-methylendioxy-amphetamine | |
| plants. | (MMDA), inter alia. | |
| Optional secondary fungal | Erinacines, hericenones, cannabidiol, | 1 ng to 500 mg |
| or plant extracts, or | cannabichromene, cannabigerol, Δ8- | |
| purified compounds | tetrahydrocannabinol, Δ9- | |
| thereof. | tetrahydrocannabinol, cannabinol, | |
| | tetrahydrocannabivarin, cannabidiol-2′,6′- | |
| | dimethyl ether, Ketamine, Antrodia, | |
| | Beauveria, Copelandia, Cordyceps, | |
| | Fomitopsis, Ganoderma, Grifola, Hericium, | |
| | Hypsizygus, Inonotus, Isaria, Panaeolus, | |
| | Phellinus, Phellinus, Piptoporus, Pleurotus, | |
| | Polyporus, Pochonia chlamydosporia, or | |
| | Trametesspecies or combinations thereof; | |
| | Bacopa monnien, Cantella asiatica, Gingko | |
| | biloba, Zingiber officinale, Ocimum sanctum, | |
| | Polygonum cuspidatum, Origanum vulgare, | |
| | Origanum onites, Rosmarinus officinalis, | |
| | Rosmarinus eriocalyx, Curcuma longa, | |
| | Camellia sinensis, Psychotria viridis, inter | |
| | alia. | |
| Optional mushroom | Hericium erinaceus (Lion's mane) | 50 mg to 2000 mg |
| mycelium and/or fruit | | |
| body mixture | | |
| Optional MAO inhibitor | β-carbolines (e.g., harmane, harmine, | 1 ng to 200 mg |
| compounds | norharmine, perlolyrine, harmol, cordysinin, | |
| | inter alia) | |
| Optional adversive | Niacin, capsaicin, ipecac, apomorphine, | 10 μg to 200 mg |
| | bittering agents (e.g., denatonium benzoate) | |
| | inter alia | |
| Optional pharmaceutical | Fillers, binders, diluents, vehicles, lubricants, | quantum sufficit |
| excipients | preservatives, flavors, colors, etc. | |

Compositions may contain one or more species or combinations of any of the species listed above. Compositions can be liquid, suspensions, emulsions, dry powder admixtures, or combinations thereof.

One embodiment described herein is a composition comprising one or more tryptamines such as psilocybin (4-phosphoryloxy-N,N-dimethyltryptamine), baeocystin, (4-phosphoryloxy-N-methyltryptamine), norbaeocystin (4-phosphoryloxy-tryptamine), psilocin (4-hydroxy-N,N-dimethyltryptamine, norpsilocin (4-hydroxy-N-methyl-tryptamine), N,N-dimethyltryptamine, 4-hydroxytryptamine, inter alia, in pure form or comprising extracts from Psilocybe and psilocybin containing mushrooms, or combinations thereof.

Another embodiment is a composition of one or more tryptamines or in pure form or extracts from psilocybin containing mushrooms, or combinations thereof combined with one or more erinacines or hericenones in pure form, extracts from Hericium mushroom species, or combinations thereof.

Another embodiment is a composition of one or more tryptamines or in pure form or extracts from psilocybin containing mushrooms, or combinations thereof, and further combined with one or more adversive compounds such as niacin, ipecac, apomorphine, bittering agents (e.g., denatonium benzoate), capsaicin, capsacutin dihydrocapsaicin, nordihydrocapsaicin, homocapsaicin, homodihydrocapsaicin, capsaicinoids, gingerol, pipeline, isopiperine, zingerone, shogaol, vanillylamide derivatives, or combinations thereof, inter alia.

Another embodiment is a composition of one or more tryptamines or in pure form or extracts from psilocybin containing mushrooms, or combinations thereof combined with one or more erinacines or hericenones in pure form, extracts from Hericium mushroom species (e.g., H. erinaceus, H. coralloides, H. ramosum) or combinations thereof, and further combined with one or more adversive compounds such as niacin, capsaicin, ipecac, apomorphine, bittering agents (e.g., denatonium benzoate), inter alia.

Another embodiment is a composition of one or more tryptamines or in pure form or extracts from psilocybin mushrooms containing fungi, extracts thereof or pure chemicals thereof; or plant extracts, or pure chemicals thereof; or combinations thereof.

Another embodiment is a composition of one or more tryptamines or in pure form or extracts from psilocybin containing mushrooms, or combinations thereof combined with one or more monoamine oxidase (MAO) inhibitors, such as β-carbolines (e.g., harmane, harmine, norharmine, perlolyrine, harmol, cordysinin, inter alia), to any of the above mentioned compositions to enhance the pharmaceutical efficacy of the tryptamine(s).

Pharmaceutical excipients useful for the compositions as described herein comprise: acidifying agents (acetic acid, glacial acetic acid, citric acid, fumaric acid, hydrochloric acid, diluted hydrochloric acid, malic acid, nitric acid, phosphoric acid, diluted phosphoric acid, sulfuric acid, tartaric acid); alkalizing agents (ammonia solution, ammonium carbonate, diethanolamine, diisopropanolamine, potassium hydroxide, sodium bicarbonate, sodium borate, sodium carbonate, sodium hydroxide, trolamine); antifoaming agents (dimethicone, simethicone); antimicrobial preservatives (benzalkonium chloride, benzalkonium chloride solution, benzethonium chloride, benzoic acid, benzyl alcohol, butylparaben, cetylpyridinium chloride, chlorobutanol, chlorocresol, cresol, dehydroacetic acid, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, ascorbic acid, thimerosal, thymol); antioxidants (ascorbic acid, ascorbyl palmitate, butylated hydroxyanisole, butylated hydroxytoluene, hypophosphorous acid, monothioglycerol, propyl gallate, sodium formaldehyde sulfoxylate, sodium metabisulfite, sodium thiosulfate, sulfur dioxide, tocopherol, tocopherols excipient); buffering agents (acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate); chelating agents (edetate disodium, ethylenediaminetetraacetic acid and salts, edetic acid); coating agents (sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyvinyl acetate phthalate, shellac, sucrose, titanium dioxide, carnauba wax, microcrystalline wax, zein); colorants (caramel, red, yellow, black or blends, ferric oxide); complexing agents (ethylenediaminetetraacetic acid and salts (EDTA), edetic acid, gentisic acid ethanolamide, oxyquinoline sulfate); desiccants (calcium chloride, calcium sulfate, silicon dioxide); emulsifying and/or solubilizing agents (acacia, cholesterol, diethanolamine (adjunct), glyceryl monostearate, lanolin alcohols, mono- and di-glycerides, monoethanolamine (adjunct), lecithin, oleic acid (adjunct), oleyl alcohol (stabilizer), poloxamer, polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, diacetate, monostearate, sodium lauryl sulfate, sodium stearate, sorbitan monolaurate, sorbitan monooleate, sorbitan monopalmitate, sorbitan monostearate, stearic acid, trolamine, emulsifying wax); filtering aids (powdered cellulose, purified siliceous earth); flavors and perfumes (anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, vanillin); humectants (glycerol, hexylene glycol, sorbitol); plasticizers (e.g., castor oil, diacetylated monoglycerides, diethyl phthalate, glycerol, mono- and di-acetylated monoglycerides, propylene glycol, triacetin, triethyl citrate); polymers (e.g., cellulose acetate, alkyl celluloses, hydroxyalkyl, acrylic polymers and copolymers); solvents (acetone, alcohol, diluted alcohol, amylene hydrate, benzyl benzoate, butyl alcohol, carbon tetrachloride, chloroform, corn oil, cottonseed oil, ethyl acetate, glycerol, hexylene glycol, isopropyl alcohol, methyl alcohol, methylene chloride, methyl isobutyl ketone, mineral oil, peanut oil, propylene carbonate, sesame oil, water for injection, sterile water for injection, sterile water for irrigation, purified water); sorbents (powdered cellulose, charcoal, purified siliceous earth); carbon dioxide sorbents (barium hydroxide lime, soda lime); stiffening agents (hydrogenated castor oil, cetostearyl alcohol, cetyl alcohol, cetyl esters wax, hard fat, paraffin, polyethylene excipient, stearyl alcohol, emulsifying wax, white wax, yellow wax); suspending and/or viscosity-increasing agents (acacia, agar, alginic acid, aluminum monostearate, bentonite, purified bentonite, magma bentonite, carbomer, carboxymethylcellulose calcium, carboxymethylcellulose sodium, carboxymethylcellulose sodium carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, alginate, silicon dioxide, colloidal silicon dioxide, sodium alginate, tragacanth, xanthan gum); sweetening agents (aspartame, dextrates, dextrose, excipient dextrose, fructose, mannitol, saccharin, calcium saccharin, sodium saccharin, sorbitol, solution sorbitol, sucrose, compressible sugar, confectioner's sugar, syrup); surfactants (simethicone); tablet binders (acacia, alginic acid, sodium carboxymethylcellulose, microcrystalline cellulose, dextrin, ethylcellulose, gelatin, liquid glucose, guar gum, hydroxypropyl methylcellulose, methylcellulose, polyethylene oxide, povidone, pregelatinized starch, syrup); tablet and/or capsule diluents (calcium carbonate, dibasic calcium phosphate, tribasic calcium phosphate, calcium sulfate, microcrystalline cellulose, powdered cellulose, dextrates, dextrin, dextrose excipient, fructose, kaolin, lactose, mannitol, sorbitol, starch, pregelatinized starch, sucrose, compressible sugar, confectioner's sugar); tablet disintegrants (alginic acid, microcrystalline cellulose, croscarmellose sodium, crospovidone, polacrilin potassium, sodium starch glycolate, starch, pregelatinized starch); tablet and/or capsule lubricants (calcium stearate, glyceryl behenate, magnesium stearate, light mineral oil, sodium stearyl fumarate, stearic acid, purified stearic acid, talc, hydrogenated vegetable oil, zinc stearate); thickening agents (gelatin having a bloom strength of 50-100); tonicity agent (dextrose, glycerol, mannitol, potassium chloride, sodium chloride); vehicle: flavored and/or sweetened (aromatic elixir, compound benzaldehyde elixir, iso-alcoholic elixir, peppermint water, sorbitol solution, syrup, tolu balsam syrup); vehicle: oleaginous (almond oil, corn oil, cottonseed oil, ethyl oleate, isopropyl myristate, isopropyl palmitate, mineral oil, light mineral oil, myristyl alcohol, octyl dodecanol, olive oil, peanut oil, persic oil, sesame oil, soybean oil, squalane); vehicle: solid carrier (sugar spheres); vehicle: sterile (bacteriostatic water for injection, bacteriostatic sodium chloride injection); viscosity-increasing (see suspending agent); water repelling agents (cyclomethicone, dimethicone, simethicone); and/or solubilizing agent (benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20, cetostearyl ether, polyoxyl 40 stearate, polysorbate 20, polysorbate 40, polysorbate 60, polysorbate 80, sodium lauryl sulfate, sorbitan monolaurate, sorbitan monooleate, sorbitan monopalmitate, sorbitan monostearate, tyloxapol). This list is not meant to be exclusive, but instead merely representative of the classes of excipients and the particular excipients that may be used in oral dosage forms as described herein. See Remington's Essentials of Pharmaceutics, Pharmaceutical Press Publishing Company, London, UK, 1st Edition, 2013, and the Handbook of Pharmaceutical Excipients, 8th Edition, Pharmaceutical Press Publishing Company London, UK, 2017, each of which is incorporated by reference herein for such teachings.

### Dosages

Toxicity and therapeutic efficacy of compounds described herein, including pharmaceutically acceptable salts and deuterated variants, can be determined by standard pharmaceutical procedures in cell cultures or experimental animals. The LD₅₀ is the dose lethal to 50% of the population. The ED₅₀ is the dose therapeutically effective in 50% of the population. The dose ratio between toxic and therapeutic effects (LD₅₀/ED₅₀) is the therapeutic index. Compounds that exhibit large therapeutic indexes are preferred. While compounds that exhibit toxic side effects may be used, care should be taken to design a delivery system that targets such compounds to the site of affected tissue in order to minimize potential damage to uninfected cells and thereby reduce side effects.

Data obtained from the cell culture assays and animal studies can be used in formulating a range of dosage for use in humans. The dosage of such compounds may lie within a range of circulating concentrations that include the ED₅₀ with little or no toxicity. The dosage may vary within this range depending upon the dosage form employed and the route of administration utilized. For any compound, the therapeutically effective dose can be estimated initially from cell culture assays. A dose may be formulated in animal models to achieve a circulating plasma concentration range that includes the ICso (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

In one embodiment, the pharmaceutical compositions described herein provide a dosage form of the pharmaceutical compositions described here for administration to a subject. In one embodiment, the subject is suffering from or has the symptoms of one or more neurologic diseases or disorders or wishes to enhance one or more cognitive or sensory motor traits. The dosage form can be administered, for example, to a subject, or a subject in need thereof. In one aspect, the subject is a mammal, or a mammal in need thereof. In one aspect, the subject is a human, or human in need thereof. In one aspect, the subject is a human or a human in need thereof. In one aspect, the subject is a child (˜0-9 years old) or an adolescent (˜10-17 years old). In one aspect, the subject is from about 0 to about 9 years of age. In another aspect, the subject is from about 10 years to about 17 years of age. In another aspect, the subject is over 17 years of age. In another aspect, the subject is an adult (≥18 years of age).

One or more dosage forms of the compositions described herein can be administered, for example, 1×, 2×, 3×, 4×, 5×, 6×, or even more times per day. One or more dosage forms can be administered, for example, for 1, 2, 3, 4, 5, 6, 7 days, or even longer. One or more dosage forms can be administered, for example, for 1, 2, 3, 4 weeks, or even longer. One or more dosage forms can be administered, for example, for 1, 2, 3, 4, 5, 6, 7, 8, 9, 10, 11, 12 months, 1 year, 2, years, 3 years, 4 years, 5 years, over 5 years, a decade, multiple decades, or even longer. One or more dosage forms can be administered at a regular interval until the subject or subject in need thereof, does not require treatment, prophylaxis, or amelioration of any disease or condition including but not limited to a neurological or neurodegenerative disease or disorder.

In one embodiment, the compositions described herein can be administered as dosage forms in various regimens, including one dose per day (QD), two doses per day (BID), three doses per day (TID), or four times per day (QID) to achieve a total daily dosage. In another embodiment, any of the foregoing doses comprise a total daily dosage.

In one embodiment, the pharmaceutical composition comprises a dose of about 1 ng to about 100 mg of one or more tryptamines or an amount of a mushroom (or plant) extract or mushroom (or plant) having an equivalent amount of tryptamine(s). In another embodiment, the composition comprises about 1 μg to about 10 mg of one or more tryptamines or an amount of a mushroom extract or mushroom having an equivalent amount of tryptamine(s). In another embodiment, the composition comprises about 1 μg to about 100 μg of one or more tryptamines or an amount of a mushroom extract or mushroom having an equivalent amount of tryptamine(s). In another embodiment, the composition comprises about 1 μg to about 5 mg of one or more tryptamines or an amount of a mushroom extract or mushroom having an equivalent amount of tryptamine(s). In another embodiment, the composition comprises about 100 μg to about 1 mg of one or more tryptamines or an amount of a mushroom extract or mushroom having an equivalent amount of tryptamine(s). In one aspect, the composition comprises about: 1 ng, 5 ng, 10 ng, 20 ng, 30 ng, 40 ng, 50 ng, 60 ng, 70 ng, 80 ng, 90 ng, 100 ng, 110 ng, 120 ng, 130 ng, 140 ng, 150 ng, 160 ng, 170 ng, 180 ng, 190 ng, 200 ng, 210 ng, 220 ng, 230 ng, 240 ng, 250 ng, 260 ng, 270 ng, 280 ng, 290 ng, 300 ng, 310 ng, 320 ng, 330 ng, 340 ng, 350 ng, 360 ng, 370 ng, 380 ng, 390 ng, 400 ng, 410 ng, 420 ng, 430 ng, 440 ng, 450 ng, 460 ng, 470 ng, 480 ng, 490 ng, 500 ng, 510 ng, 520 ng, 530 ng, 540 ng, 550 ng, 560 ng, 570 ng, 580 ng, 590 ng, 600 ng, 610 ng, 620 ng, 630 ng, 640 ng, 650 ng, 660 ng, 670 ng, 680 ng, 690 ng, 700 ng, 710 ng, 720 ng, 730 ng, 740 ng, 750 ng, 760 ng, 770 ng, 780 ng, 790 ng, 800 ng, 810 ng, 820 ng, 830 ng, 840 ng, 850 ng, 860 ng, 870 ng, 880 ng, 890 ng, 900 ng, 910 ng, 920 ng, 930 ng, 940 ng, 950 ng, 960 ng, 970 ng, 980 ng, 990 ng, 1 μg, 5 μg, 10 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, 300 μg, 310 μg, 320 μg, 330 μg, 340 μg, 350 μg, 360 μg, 370 μg, 380 μg, 390 μg, 400 μg, 410 μg, 420 μg, 430 μg, 440 μg, 450 μg, 460 μg, 470 μg, 480 μg, 490 μg, 500 μg, 510 μg, 520 μg, 530 μg, 540 μg, 550 μg, 560 μg, 570 μg, 580 μg, 590 μg, 600 μg, 610 μg, 620 μg, 630 μg, 640 μg, 650 μg, 660 μg, 670 μg, 680 μg, 690 μg, 700 μg, 710 μg, 720 μg, 730 μg, 740 μg, 750 μg, 760 μg, 770 μg, 780 μg, 790 μg, 800 μg, 810 μg, 820 μg, 830 μg, 840 μg, 850 μg, 860 μg, 870 μg, 880 μg, 890 μg, 900 μg, 910 μg, 920 μg, 930 μg, 940 μg, 950 μg, 960 μg, 970 μg, 980 μg, 990 μg, or 1000 μg of one or more tryptamines or an amount of a mushroom extract or mushroom having an equivalent amount of tryptamine(s). In another aspect, the composition comprises about: 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, 5.0 mg, 5.1 mg, 5.2 mg, 5.3 mg, 5.4 mg, 5.5 mg, 5.6 mg, 5.7 mg, 5.8 mg, 5.9 mg, 6.0 mg, 6.1 mg, 6.2 mg, 6.3 mg, 6.4 mg, 6.5 mg, 6.6 mg, 6.7 mg, 6.8 mg, 6.9 mg, 7.0 mg, 7.1 mg, 7.2 mg, 7.3 mg, 7.4 mg, 7.5 mg, 7.6 mg, 7.7 mg, 7.8 mg, 7.9 mg, 8.0 mg, 8.1 mg, 8.2 mg, 8.3 mg, 8.4 mg, 8.5 mg, 8.6 mg, 8.7 mg, 8.8 mg, 8.9 mg, 9.0 mg, 9.1 mg, 9.2 mg, 9.3 mg, 9.4 mg, 9.5 mg, 9.6 mg, 9.7 mg, 9.8 mg, 9.9 mg, or 10.0 mg of one or more tryptamines or an amount of a mushroom extract or mushroom having an equivalent amount of tryptamine(s).

In one embodiment, the dose of tryptamine is about 0.00001 mg/kg to about 0.2 mg/kg, assuming an average mass of 70 kg for a human. In one embodiment, the dose of tryptamine is 0.0001 mg/kg to about 0.001 mg/kg. In another embodiment, the dose of tryptamine is 0.001 mg/kg to about 0.01 mg/kg. In another embodiment, the dose of tryptamine is 0.01 mg/kg to about 0.1 mg/kg. In another embodiment, the dose of tryptamine is 0.1 mg/kg to about 0.2 mg/kg.

In another embodiment, the dose of tryptamine is about 0.005 mg/kg, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.10 mg/kg, 0.11 mg/kg, 0.12 mg/kg, 0.13 mg/kg, 0.14 mg/kg, 0.15 mg/kg, 0.16 mg/kg, 0.17 mg/kg, 0.18 mg/kg, 0.19 mg/kg, 0.20 mg/kg, In another embodiment, the dose of tryptamine is about 0.01 mg/kg to about 0.05 mg/kg. In another embodiment, the dose of tryptamine is about 0.01 mg/kg to about 0.02 mg/kg.

In one embodiment, the dose of the erinacines, hericenones, one or more adversive compounds such as niacin, capsaicin, ipecac, apomorphine, bittering agents, or an amount of a mushroom or plant extract or mushroom or plant having an equivalent amount of about 1 ng, 5 ng, 10 ng, 20 ng, 30 ng, 40 ng, 50 ng, 60 ng, 70 ng, 80 ng, 90 ng, 100 ng, 110 ng, 120 ng, 130 ng, 140 ng, 150 ng, 160 ng, 170 ng, 180 ng, 190 ng, 200 ng, 210 ng, 220 ng, 230 ng, 240 ng, 250 ng, 260 ng, 270 ng, 280 ng, 290 ng, 300 ng, 310 ng, 320 ng, 330 ng, 340 ng, 350 ng, 360 ng, 370 ng, 380 ng, 390 ng, 400 ng, 410 ng, 420 ng, 430 ng, 440 ng, 450 ng, 460 ng, 470 ng, 480 ng, 490 ng, 500 ng, 510 ng, 520 ng, 530 ng, 540 ng, 550 ng, 560 ng, 570 ng, 580 ng, 590 ng, 600 ng, 610 ng, 620 ng, 630 ng, 640 ng, 650 ng, 660 ng, 670 ng, 680 ng, 690 ng, 700 ng, 710 ng, 720 ng, 730 ng, 740 ng, 750 ng, 760 ng, 770 ng, 780 ng, 790 ng, 800 ng, 810 ng, 820 ng, 830 ng, 840 ng, 850 ng, 860 ng, 870 ng, 880 ng, 890 ng, 900 ng, 910 ng, 920 ng, 930 ng, 940 ng, 950 ng, 960 ng, 970 ng, 980 ng, 990 ng, or 1000 ng; 1 μg, 5 μg, 10 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, 300 μg, 310 μg, 320 μg, 330 μg, 340 μg, 350 μg, 360 μg, 370 μg, 380 μg, 390 μg, 400 μg, 410 μg, 420 μg, 430 μg, 440 μg, 450 μg, 460 μg, 470 μg, 480 μg, 490 μg, 500 μg, 510 μg, 520 μg, 530 μg, 540 μg, 550 μg, 560 μg, 570 μg, 580 μg, 590 μg, 600 μg, 610 μg, 620 μg, 630 μg, 640 μg, 650 μg, 660 μg, 670 μg, 680 μg, 690 μg, 700 μg, 710 μg, 720 μg, 730 μg, 740 μg, 750 μg, 760 μg, 770 μg, 780 μg, 790 μg, 800 μg, 810 μg, 820 μg, 830 μg, 840 μg, 850 μg, 860 μg, 870 μg, 880 μg, 890 μg, 900 μg, 910 μg, 920 μg, 930 μg, 940 μg, 950 μg, 960 μg, 970 μg, 980 μg, 990 μg, or 1000 μg; 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, 5.0 mg, 5.1 mg, 5.2 mg, 5.3 mg, 5.4 mg, 5.5 mg, 5.6 mg, 5.7 mg, 5.8 mg, 5.9 mg, 6.0 mg, 6.1 mg, 6.2 mg, 6.3 mg, 6.4 mg, 6.5 mg, 6.6 mg, 6.7 mg, 6.8 mg, 6.9 mg, 7.0 mg, 7.1 mg, 7.2 mg, 7.3 mg, 7.4 mg, 7.5 mg, 7.6 mg, 7.7 mg, 7.8 mg, 7.9 mg, 8.0 mg, 8.1 mg, 8.2 mg, 8.3 mg, 8.4 mg, 8.5 mg, 8.6 mg, 8.7 mg, 8.8 mg, 8.9 mg, 9.0 mg, 9.1 mg, 9.2 mg, 9.3 mg, 9.4 mg, 9.5 mg, 9.6 mg, 9.7 mg, 9.8 mg, 9.9 mg, or 10.0 mg; 0.1 mg, 0.25 mg, 0.5 mg, 1.0 mg, 2.5 mg, 5.0 mg, 10.0 mg, 20.0 mg, 30.0 mg, 40.0 mg, 50.0 mg, 60.0 mg, 70.0 mg, 80.0 mg, 90.0 mg, or 100.0 mg; 1 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, or 500 mg of compound.

In one embodiment, the dose of the erinacines, hericenones, one or more adversive compounds such as niacin, capsaicin, ipecac, apomorphine, bittering agents, or an amount of a mushroom or plant extract or mushroom or plant having an equivalent amount of about 0.1 mg/kg, 0.25 mg/kg, 0.5 mg/kg, 0.75 mg/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg.

In one embodiment, the dose of the monoamine oxidase inhibitor, such as β-carbolines (e.g., harmane, harmine, norharmine, perlolyrine, harmol, cordysinin, inter alia) ranges from 70 mg to 200 mg.

In one embodiment, the dose of Hericium erinaceus (Lion's mane) mycelium ranges from 50 mg to 2000 mg. In one aspect, the dose of Hericium erinaceus (Lion's mane) mycelium ranges from 400 mg to 500 mg.

In one embodiment, the pharmaceutical composition comprises:
0.001 mg to 0.01 mg, 0.01 mg to 0.1 mg, 0.01 mg to 1 mg, 0.1 mg to 5 mg, 0.1 mg to 1 mg, 0.5 mg to 1 mg, 0.5 mg to 5 mg, 0.25 mg to 1 mg, 0.2 mg to 2 mg, 0.2 mg to 5 mg of one or more tryptamines or an amount of a plant or mushroom extract or plant or mushroom to provide an equivalent dose; and/or
1 μg to 5 μg, 1 μg to 10 μg, 5 μg to 10 μg, 10 μg to 5 mg, 10 μg to 100 μg, 100 μg to 1 mg, 500 μg to 1 mg, 500 μg to 5 mg, 1 mg to 5 mg, 100 μg to 1 mg, 100 μg to 500 μg, 100 μg to 250 μg; 250 μg to 1 mg; 750 μg to 1 mg, 250 μg to 750 μg, erinacines or hericenones or an amount of a plant or mushroom extract or plant or mushroom to provide an equivalent dose; and/or
0.1 mg to 10 mg, 1 mg to 500 mg, 1 mg to 100 mg, 200 mg to 500 mg, 50 mg to 200 mg, 10 mg to 50 mg, 50 mg to 200 mg, 1 mg to 200 mg, 1 mg to 50 mg of adversive; and/or
50 mg to 2000 mg, 400 mg to 500 mg of Hericium erinaceus (Lion's mane) mycelium;

and/or
70 mg to 200 mg monoamine oxidase inhibitor.

The percent mass of psilocybin, psilocin, and baeocystin in dried Psilocybe mushrooms is shown below in Table 4.

TABLE 4
Psilocybin, psilocin, and baeocystin concentrations
in Psilocybe mushrooms

| | Mass percent based on dry | | |
|---|---|---|---|
| | weight of mushroom | | |
| Species | Psilocybin | Psilocin | Baeocystin |
| P. azurescens | 1.78 | 0.38 | 0.35 |
| P. bohemica | 1.34 | 0.11 | 0.02 |
| P. semilanceata | 0.98 | 0.02 | 0.36 |
| P. baeocystis | 0.85 | 0.59 | 0.10 |
| P. cyanescens | 0.85 | 0.36 | 0.03 |
| P. tampanensis | 0.68 | 0.32 | n/d |
| P. cubensis | 0.63 | 0.60 | 0.025 |
| P. weilii | 0.61 | 0.27 | 0.05 |
| P. hoogshagenii | 0.60 | 0.10 | n/d |
| P. stuntzii | 0.36 | 0.12 | 0.02 |
| P. cyanofibrillosa | 0.21 | 0.04 | n/d |
| P. liniformans | 0.16 | n/d | 0.05 |
| Average | 0.754% | 0.243% | 0.137% |

Data from Stamets, Psilocybin Mushrooms of the World, Ten Speed Press, page 39 (1996)

Table 5 shows the relative amount of psilocybin, psilocin, and baeocystin in dried Psilocybe mushrooms.

TABLE 5
Relative amounts of psilocybin, psilocin,
baeocystin in dry Psilocybe mushrooms

| | Psilocybin | Psilocin | Baeocystin |
|---|---|---|---|
| Dry Mushroom | 0.754% | 0.243% | 0.137% |
| g | mg | mg | mg |
| 0.2-0.8 | 1.5-6.0 | 0.5-1.9 | 0.3-1.1 |
| 0.8-1.0 | 6.0-7.5 | 1.9-2.4 | 1.1-1.4 |
| 1.0-1.5 | 7.5-11.3 | 2.4-36 | 1.4-2.1 |
| 1.5-3.0 | 11.3-22.6 | 3.6-7.3 | 2.1-4.1 |
| 3.0-4.0 | 22.6-30.2 | 7.3-9.7 | 4.1-5.5 |
| 4.0-5.0 | 30.2-37.7 | 9.7-12.2 | 5.5-6.9 |

Based on data from Stamets, Psilocybin Mushrooms of the World, Ten Speed Press, page 39 (1996)

### Methods of Treatment

Methods for treating, prophylaxis of, or ameliorating symptoms of an infectious disease including administering an effective amount of the compositions detailed herein are contemplated. Methods for treating, prophylaxis of, or ameliorating symptoms of a bacterial or viral infection or modulating a bacterial or viral infection that includes administering an effective amount of one or more of tryptamines, erinacines, hericenones, or pharmaceutically acceptable salts, hydrates, solvates, prodrugs, stereoisomers, or tautomers thereof, or combinations thereof are also contemplated. Another embodiment described herein is a method of treating a subject suffering from or having the symptoms of an infectious disease or disorder by orally administering one or more of the pharmaceutical compositions described herein to the subject. The composition may be administered in one or more doses, one or more times per day for a total daily dosage. In one aspect, the compositions described herein are effective to at least partially treat, alleviate, prevent, or ameliorate symptoms of an infectious disease. Further, provided herein are means for modulating an inflammatory response that includes administering to a subject an effective amount of the composition described herein.

In some embodiments, a dose of the composition may be administered to the subject 1 time per day, 2 times per day, 3 times per day, 4 times per day, or 5 times per day. In some embodiments, the dose includes at least 1 capsule, at least 2 capsules, at least 3 capsules, at least 4 capsules, at least 5 capsules, at least 6 capsules, at least 7 capsules, at least 8 capsules. In some embodiments, the composition may be administered to the subject for about 1 to 30 consecutive days, about 5 to 30 consecutive days, about 10 to 30 consecutive days, about 15 to 30 consecutive days, about 1 to 15 consecutive days, about 5 to 15 consecutive days, or about 10 to 15 consecutive days. In some embodiments, the composition may be administered to the subject for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days.

For example, administration of the composition to the subject may result in inhibition or slowing of the infectious disease. In another example, administration of the composition to the subject may result in inhibition or slowing of the normal rate of increase of viral load as compared to an untreated subject. As used herein, the term “viral load” is a measurement of the amount of a virus in a subject. In some embodiments, the infectious disease may cause lung inflammation. The lung inflammation may be associated with, but not limited to, respiratory failure, respiratory distress, pulmonary disease, cystic fibrosis, asthma, bronchitis, inflammation/swelling of the lungs, chronic obstructive pulmonary disease (COPD), pneumonia, restrictive lung disease, bronchiectasis, pulmonary fibrosis, sarcoidosis, allergies, smoking, emphysema, acute respiratory distress syndrome (ARDS), interstitial lung disease (ILD), pneumoconiosis or lung cancer. The lung diseases may affect the alveoli, trachea, interstitium, pluera, bronchi and/or bronchioles. The lung disease may cause diffuse alveolar damage, denuded alveolar lining cells with reactive type II pneumocyte hyperplasia, intra-alveolar fibrinous exudates, loose interstitial fibrosis, intra-alveolar loose fibrous plugs of organizing pneumonia, intra-alveolar organizing fibrin, damaged alveolar epithelial cells, desquamated cells within the alveolar space, cellular fibromyxoid exudates, desquamation of pneumocytes, hyaline membrane formation (e.g. indication of ARDS), pulmonary oedema, (e.g. early-phase ARDS). The lung disease may also cause chronic inflammation such as interstitial mononuclear inflammatory infiltrates dominated by lymphocytes. The lung disease may cause infiltration of the intra-alveolar spaces in the lung by multinucleated syncytial cells with atypical enlarged pneumocytes characterized by large nuclei, amphophilic granular cytoplasm, and/or prominent nucleoli that show viral cytopathic-like changes. The lung disease may cause increased inflammatory FCN1+ macrophages that replace FABP4+ macrophages in severe disease. The lung disease may cause highly expanded and functional competent tissue resident clonal CD8+ T cells in mild disease. Blood vessels or interstitial areas between alveoli may not be affected by the lung disease. In some embodiments, the infectious disease may include one or more symptoms such as shortness of breath, wheezing, coughing, yellow mucus, green mucus, blood-tinged mucus, chest pain, breathlessness, rapid breathing, hypoxia, inflammation of the lung tissue, rapid heart rate, or increased blood pressure, or decreased blood pressure. In some embodiments, the subject may have COPD, cardiovascular disease, diabetes mellitus, hypertension, or a combination thereof. In some embodiments, the subject may be at least 1 year old, at least 2 years old, at least 3 years old, at least 4 years old, at least 5 years old, at least 6 years old, at least 7 years old, at least 8 years old, at least 9 years old, at least 10 years old, at least 11 years old, at least 12 years old, at least 13 years old, at least 14 years old, at least 15 years old, at least 16 years old, at least 17 years old, at least 18 years old, at least 19 years old, at least 20 years old, at least 21 years old, at least 22 years old, at least 23 years old, at least 24 years old, at least 25 years old, at least 26 years old, at least 27 years old, at least 28 years old, at least 29 years old, at least 30 years old, at least 31 years old, at least 32 years old, at least 33 years old, at least 34 years old, at least 35 years old, at least 36 years old, at least 37 years old, at least 38 years old, at least 39 years old, at least 40 years old, at least 41 years old, at least 42 years old, at least 43 years old, at least 44 years old, at least 45 years old, at least 46 years old, at least 47 years old, at least 48 years old, at least 49 years old, at least 50 years old, at least 51 years old, at least 52 years old, at least 53 years old, at least 54 years old, at least 55 years old, at least 56 years old, at least 57 years old, at least 58 years old, at least 59 years old, at least 60 years old, at least 61 years old, at least 62 years old, at least 63 years old, at least 64 years old, at least 65 years old, at least 66 years old, at least 67 years old, at least 68 years old, at least 69 years old, at least 70 years old, at least 71 years old, at least 72 years old, at least 73 years old, at least 74 years old, at least 75 years old, at least 76 years old, at least 77 years old, at least 78 years old, at least 79 years old, at least 80 years old, at least 81 years old, at least 82 years old, at least 83 years old, at least 84 years old, at least 85 years old, at least 86 years old, at least 87 years old, at least 88 years old, at least 89 years old, at least 90 years old, at least 91 years old, at least 92 years old, at least 93 years old, at least 94 years old, at least 95 years old, at least 96 years old, at least 97 years old, at least 98 years old, at least 99 years old, at least 100 years old, or even older.

In some embodiments, the infectious disease or condition may increase expression of growth factors. In some embodiments, the growth factors may be basic fibroblast growth factor and/or vascular endothelial growth factor. In some embodiments, the methods herein comprise administering a therapy for one of the symptoms or conditions associated with cytokine storm. For instance, if the subject develops coagulopathy, the method may comprise administering cryoprecipitate. In some embodiments, if the subject develops cardiovascular dysfunction, the method may comprise administering vasoactive infusion support. In some embodiments, if the subject develops distributive shock, the method may comprise administering alpha-agonist therapy. In some embodiments, if the subject develops cardiomyopathy, the method may comprise administering milrinone therapy. In some embodiments, if the subject develops respiratory failure, the method may comprise performing mechanical ventilation (e.g., invasive mechanical ventilation or noninvasive mechanical ventilation). In some embodiments, if the subject develops shock, the method may comprise administering crystalloid and/or colloid fluids.

In the absence of prompt intervention, such as that provided herein, a cytokine storm can result in permanent lung damage and, in many cases, death. The end stage symptoms of the cytokine storm include but are not limited to hypotension, tachycardia, dyspnea, fever, ischemia or insufficient tissue perfusion, uncontrollable hemorrhage, severe metabolism dysregulation, and multisystem organ failure. Deaths from infectious diseases such as COVID-19, are not caused by the virus itself, but rather, the cytokine storm that causes uncontrollable hemorrhaging; severe metabolism dysregulation; hypotension; tachycardia; dyspnea; fever; ischemia or insufficient tissue perfusion; and multisystem organ failure.

### Methods of Use

Another embodiment is a method of treating or preventing an infectious disease or disorder in a subject in need thereof, the method comprising administering to the subject a therapeutically effective amount of an extract, a compound, or formulation disclosed herein.

Another embodiment is a composition comprising a tryptamine, an erinacine, a hericenone, or a pharmaceutically acceptable salt, hydrate, solvate, prodrug, stereoisomer, or tautomer thereof, or a combination thereof, and a pharmaceutically acceptable carrier, for use in treating an infectious disease or disorder in a subject in need thereof.

Another embodiment is the use of a composition comprising a tryptamine, an erinacine, a hericenone, or a pharmaceutically acceptable salt, hydrate, solvate, prodrug, stereoisomer, or tautomer thereof, or a combination thereof, in the manufacture of a medicament for treating an infectious disease or disorder.

Another embodiment is the use of a pharmaceutical composition comprising a tryptamine, an erinacine, a hericenone, or a pharmaceutically acceptable salt, hydrate, solvate, prodrug, stereoisomer, or tautomer thereof, or a combination thereof, and a pharmaceutically acceptable carrier, in the manufacture of a medicament for treating an infectious disease in a subject in need thereof.

Another embodiment is a method for treating or preventing an infectious disease or disorder in a subject in need thereof comprising administering a composition comprising a tryptamine, an erinacine, a hericenone, or a pharmaceutically acceptable salt, hydrate, solvate, prodrug, stereoisomer, or tautomer thereof, or a combination thereof to the subject.

Another embodiment is the addition of one or more adversive compounds such as niacin, ipecac, apomorphine, bittering agents (e.g., denatonium benzoate), capsaicin, capsacutin dihydrocapsaicin, nordihydrocapsaicin, homocapsaicin, homodihydrocapsaicin, capsaicinoids, gingerol, pipeline, isopiperine, zingerone, shogaol, vanillylamide derivatives, or combinations thereof, inter alia.

Another embodiment is the addition of a monoamine oxidase inhibitor, such as β-carbolines (e.g., harmane, harmine, nor harmine, perlolyrine, harmol, cordysinin, inter alia), to any of the above mentioned compositions or methods to enhance the pharmaceutical efficacy of the tryptamine(s).

### Methods of Manufacturing

Another embodiment is a method of manufacturing a composition comprising one or more tryptamines, erinacines, hericenones, or pharmaceutically acceptable salts, hydrates, solvates, prodrugs, stereoisomers, or tautomers thereof, or combinations thereof.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A composition comprising: one or more tryptamines, salts thereof, or combinations thereof; and extracts or isolates from Hericium erinaceus mushroom species, erinacines, hericenones, or combinations thereof.
Clause 2. The composition of clause 1, wherein the one or more tryptamines are psilocybin, psilocin, norpsilocin, baeocystin, norbaeocystin, N,N-dimethyltryptamine (DMT), or combinations thereof.
Clause 3. The composition of clause 1 or 2, wherein the composition comprises about 1 ng to about 10 mg, about 10 mg to about 100 mg, about 10 mg to about 20 mg, about 20 mg to about 50 mg, about 20 mg to about 100 mg, about 1 ng to about 20 mg, about 1 ng to about 50 mg, or about 1 ng to about 100 mg of the one or more tryptamines, salts thereof, or combinations thereof.
Clause 4. The composition of clause 2 or 3, wherein the composition comprises about 1 ng to about 2000 mg of the extracts or isolates from Hericium erinaceus mushroom species, erinacines, hericenones, or combinations thereof.
Clause 5. The composition of any one of clauses 1-4, further comprising a monoamine oxidase inhibitor.
Clause 6. The composition of clause 5, wherein the composition comprises about 70 mg to about 200 mg of the monoamine oxidase inhibitor.
Clause 7. The composition of clause 5 or 6, wherein the monoamine oxidase inhibitor is Norharman, Harmine, 1,2,3,4-tetrahydro-β-carboline-3-carboxylic acid, 1-methyl-1,2,3,4-tetrahydro-β-carboline-3-carboxylic acid, 1-methyl-2,3,4,9-tetrahydro-1H-β-carboline-1,3-dicarboxylic acid, Harmaline, N-methoxy-1-vinyl-β-carboline, ethyl 9H-β-arboline-3-carboxylate, 1-furyl-β-carboline-3-carboxylic acid, 1-[5-(methoxymethyl)-2-furyl]-9H-β-carboline-3-carboxylic acid, 6-hydroxy-3-(6-hydroxy-1H-indol-3-yl)-9H-β-carboline-4-carboxylic acid, Strictosidine, (1S)-1-{[(2S,3R,4S)-2-(β-L-glucopyranosyloxy)-5-(methoxycarbonyl)-3-vinyl-3,4-dihydro-2H-pyran-4-yl]methyl}-2,3,4,9-tetrahydro-1H-β-carboline-1,3-dicarboxylic acid, or combinations thereof.
Clause 8. A composition comprising: psilocybin, psilocin, norpsilocin, baeocystin, norbaeocystin, N,N-dimethyltryptamine (DMT), salts thereof, or combinations thereof; and an erinacine or hericenone in pure form, extracts or isolates from Hericium erinaceus mushroom species, or combinations thereof.
Clause 9. The composition of clause 8, wherein the composition comprises about 1 ng to about 10 mg, about 10 mg to about 100 mg, about 10 mg to about 20 mg, about 20 mg to about 50 mg, about 20 mg to about 100 mg, about 1 ng to about 20 mg, about 1 ng to about 50 mg, or about 1 ng to about 100 mg of the psilocybin, psilocin, norpsilocin, baeocystin, norbaeocystin, N,N-dimethyltryptamine (DMT), salts thereof, or combinations thereof.
Clause 10. The composition of clause 8 or clause 9, wherein the composition comprises about 1 ng to about 2000 mg of the erinacine or hericenone in pure form, extracts or isolates from Hericium erinaceus mushroom species, or combinations thereof.
Clause 11. The composition of any one of clauses 8-10, further comprising a monoamine oxidase inhibitor.
Clause 12. The composition of clause 11, wherein the composition comprises about 70 mg to about 200 mg of the monoamine oxidase inhibitor.
Clause 13. The composition of clause 11 or clause 12, wherein the monoamine oxidase inhibitor is Norharman, Harmine, 1,2,3,4-tetrahydro-β-carboline-3-carboxylic acid, 1-methyl-1,2,3,4-tetrahydro-β-carboline-3-carboxylic acid, 1-methyl-2,3,4,9-tetrahydro-1H-β-carboline-1,3-dicarboxylic acid, Harmaline, N-methoxy-1-vinyl-β-carboline, ethyl 9H-β-arboline-3-carboxylate, 1-furyl-β-carboline-3-carboxylic acid, 1-[5-(methoxymethyl)-2-furyl]-9H-β-carboline-3-carboxylic acid, 6-hydroxy-3-(6-hydroxy-1H-indol-3-yl)-9H-β-carboline-4-carboxylic acid, Strictosidine, (1S)-1-{[(2S,3R,4S)-2-(β-L-glucopyranosyloxy)-5-(methoxycarbonyl)-3-vinyl-3,4-dihydro-2H-pyran-4-yl]methyl}-2,3,4,9-tetrahydro-1H-β-carboline-1,3-dicarboxylic acid, or combinations thereof.
Clause 14. A method for treating or modulating an inflammatory response triggered by an infectious disease or condition, the method comprising: administering a composition to a subject in need thereof, the composition comprising: one or more tryptamines, salts thereof, or combinations thereof.
Clause 15. A method for treating or modulating an inflammatory response triggered by an infectious disease or condition, the method comprising: administering a composition to a subject in need thereof, the composition comprising: one or more tryptamines, salts thereof, or combinations thereof; and extracts or isolates from Hericium erinaceus mushroom species, erinacines, hericenones, or combinations thereof.
Clause 16. The method of clause 14 or clause 15, wherein the composition comprises about 1 ng to about 10 mg, about 10 mg to about 100 mg, about 10 mg to about 20 mg, about 20 mg to about 50 mg, about 20 mg to about 100 mg, about 1 ng to about 20 mg, about 1 ng to about 50 mg, or about 1 ng to about 100 mg of the one or more tryptamines, salts thereof, or combinations thereof.
Clause 17. The method of any one of clauses 14-16, wherein the one or more tryptamines are psilocybin, psilocin, norpsilocin, baeocystin, norbaeocystin, N,N-dimethyltryptamine (DMT), or combinations thereof.
Clause 18. The method of any one of clauses 15-17, wherein the composition comprises about 1 ng to about 2000 mg of the extracts or isolates from Hericium erinaceus mushroom species, erinacines, hericenones, or combinations thereof.
Clause 19. The method of any one of clauses 14-18, wherein the composition further comprises a monoamine oxidase inhibitor.
Clause 20. The method of clause 19, wherein the composition comprises about 70 mg to about 200 mg of the monoamine oxidase inhibitor.
Clause 21. The method of clause 19 or clause 20, wherein the monoamine oxidase inhibitor is Norharman, Harmine, 1,2,3,4-tetrahydro-β-carboline-3-carboxylic acid, 1-methyl-1,2,3,4-tetrahydro-β-carboline-3-carboxylic acid, 1-methyl-2,3,4,9-tetrahydro-1H-β-carboline-1,3-dicarboxylic acid, Harmaline, N-methoxy-1-vinyl-β-carboline, ethyl 9H-β-arboline-3-carboxylate, 1-fury)-β-carboline-3-carboxylic acid, 1-[5-(methoxymethyl)-2-furyl]-9H-β-carboline-3-carboxylic acid, 6-hydroxy-3-(6-hydroxy-1H-indol-3-yl)-9H-β-carboline-4-carboxylic acid, Strictosidine, (1S)-1-{[(2S,3R,4S)-2-(β-L-glucopyranosyloxy)-5-(methoxycarbonyl)-3-vinyl-3,4-dihydro-2H-pyran-4-yl]methyl}-2,3,4,9-tetrahydro-1H-β-carboline-1,3-dicarboxylic acid, or combinations thereof.
Clause 22. The method of any one of clauses 14-21, wherein the inflammatory response is cytokine storm.
Clause 23. The method of any one of clauses 14-22, wherein the infectious disease or condition is a viral infection, a bacterial infection, or a parasitic infection.
Clause 24. The method of clause 23, wherein the viral infection is Paramyxoviridae (respiratory syncytial virus (RSV), parainfluenza virus (PIV), metapneumovirus (MPV), enteroviruses), Picornaviridae (Rhinovirus, RV), Coronaviridae (CoV), Adenoviridae (Adenovirus), Parvoviridae (HBoV), Orthomyxoviridae (influenza A, B, C, D, Isavirus, Thogotovirus, Quaranjavirus), Herpesviridae (human herpes viruses, Varicella zoster virus, Epstein-Barr virus, cytomegalovirus), avian influenza, smallpox, pandemic influenza, or adult respiratory distress syndrome (ARDS).
Clause 25. The method of clause 23, wherein the bacterial infection is Streptococcus pneumoniae, Mycobacterium tuberculosis, Bordetella pertussis, Haemophilus influenzae, Moraxella catarrhalis, Pseudomonas aeruginosa, Stenotrophomonas maltophila, Staphylococcus aureus, Streptococcus pyogenes, Neisseria meningitidis, Klebsiella pneumoniae, or Non-tuberculosis Mycobacterium.
Clause 26. The method of clause 23, wherein the parasitic infection is malaria.
Clause 27. The method of any one of clauses 14-26, wherein inflammation is reduced and neuroregeneration is induced in the subject.
Clause 28. The method of clause 27, wherein neuroregeneration comprises neurite outgrowth.
Clause 29. A method for inducing expression of an anti-inflammatory cytokine, the method comprising administering a composition to a subject in need thereof, the composition comprising: one or more tryptamines, salts thereof, or combinations thereof.
Clause 30. A method for inducing expression of an anti-inflammatory cytokine, the method comprising administering a composition to a subject in need thereof, the composition comprising: one or more tryptamines, salts thereof, or combinations thereof; and extracts or isolates from Hericium erinaceus mushroom species, erinacines, hericenones, or combinations thereof.
Clause 31. The method of clause 29 or 30, wherein the composition comprises about 1 ng to about 10 mg, about 10 mg to about 100 mg, about 10 mg to about 20 mg, about 20 mg to about 50 mg, about 20 mg to about 100 mg, about 1 ng to about 20 mg, about 1 ng to about 50 mg, or about 1 ng to about 100 mg of the one or more tryptamines, salts thereof, or combinations thereof.
Clause 32. The method of any one of clauses 29-31, wherein the one or more tryptamines are psilocybin, psilocin, norpsilocin, baeocystin, norbaeocystin, N,N-dimethyltryptamine (DMT), or combinations thereof.
Clause 33. The method of any one of clauses 30-32, wherein the composition comprises about 1 ng to about 2000 mg of the extracts or isolates from Hericium erinaceus mushroom species, erinacines, hericenones, or combinations thereof.
Clause 34. The method of any one of clauses 29-33, wherein the composition further comprises a monoamine oxidase inhibitor.
Clause 35. The method of clause 34, wherein the composition comprises about 70 mg to about 200 mg of the monoamine oxidase inhibitor.
Clause 36. The method of clause 34 or clause 35, wherein the monoamine oxidase inhibitor is Norharman, Harmine, 1,2,3,4-tetrahydro-β-carboline-3-carboxylic acid, 1-methyl-1,2,3,4-tetrahydro-β-carboline-3-carboxylic acid, 1-methyl-2,3,4,9-tetrahydro-1H-β-carboline-1,3-dicarboxylic acid, Harmaline, N-methoxy-1-vinyl-β-carboline, ethyl 9H-β-arboline-3-carboxylate, 1-fury)-β-carboline-3-carboxylic acid, 1-[5-(methoxymethyl)-2-furyl]-9H-β-carboline-3-carboxylic acid, 6-hydroxy-3-(6-hydroxy-1H-indol-3-yl)-9H-β-carboline-4-carboxylic acid, Strictosidine, (1S)-1-{[(2S,3R,4S)-2-(β-L-glucopyranosyloxy)-5-(methoxycarbonyl)-3-vinyl-3,4-dihydro-2H-pyran-4-yl]methyl}-2,3,4,9-tetrahydro-1H-β-carboline-1,3-dicarboxylic acid, or combinations thereof.
Clause 37. The method of any one of clauses 29-36, wherein the anti-inflammatory cytokine is IL-4, IL-10, IL-1RA, or a combination thereof.
Clause 38. The method of any one of clauses 29-37, wherein inflammation is reduced and neuroregeneration is induced in the subject.
Clause 39. The method of clause 38, wherein neuroregeneration comprises neurite outgrowth.
Clause 40. A method for treating or modulating an inflammatory response triggered by an infectious disease or condition by inducing expression of one or more anti-inflammatory cytokines selected from the group of IL-4, IL-10, and IL-1RA, the method comprising: administering a composition to a subject in need thereof, the composition comprising: about 1 ng to about 10 mg, about 10 mg to about 100 mg, about 10 mg to about 20 mg, about 20 mg to about 50 mg, about 20 mg to about 100 mg, about 1 ng to about 20 mg, about 1 ng to about 50 mg, or about 1 ng to about 100 mg of one or more tryptamines, salts thereof, or combinations thereof; and about 10 ng to about 2000 mg of extracts or isolates from Hericium erinaceus mushroom species, erinacines, hericenones, or combinations thereof.
Clause 41. A method for treating or modulating an inflammatory response triggered by an infectious disease or condition by inducing expression of one or more anti-inflammatory cytokines selected from the group of IL-4, IL-10, and IL-1RA, the method comprising: administering a composition to a subject in need thereof, the composition comprising: about 1 ng to about 10 mg, about 10 mg to about 100 mg, about 10 mg to about 20 mg, about 20 mg to about 50 mg, about 20 mg to about 100 mg, about 1 ng to about 20 mg, about 1 ng to about 50 mg, or about 1 ng to about 100 mg of one or more tryptamines, salts thereof, or combinations thereof; about 1 ng to about 2000 mg of extracts or isolates from Hericium erinaceus mushroom species, erinacines, hericenones, or combinations thereof; and about 70 mg to about 200 mg of a monoamine oxidase inhibitor.
Clause 42. The method of clause 40 or 41, wherein the inflammatory response is cytokine storm.
Clause 43. The method of any one of clauses 40-42, wherein the infectious disease or condition is a viral infection, a bacterial infection, or a parasitic infection.
Clause 44. The method of clause 43, wherein the viral infection is Paramyxoviridae (respiratory syncytial virus (RSV), parainfluenza virus (PIV), metapneumovirus (MPV), enteroviruses), Picornaviridae (Rhinovirus, RV), Coronaviridae (CoV), Adenoviridae (Adenovirus), Parvoviridae (HBoV), Orthomyxoviridae (influenza A, B, C, D, Isavirus, Thogotovirus, Quaranjavirus), Herpesviridae (human herpes viruses, Varicella zoster virus, Epstein-Barr virus, cytomegalovirus), avian influenza, smallpox, pandemic influenza, or adult respiratory distress syndrome (ARDS).
Clause 45. The method of clause 43, wherein the bacterial infection is Streptococcus pneumoniae, Mycobacterium tuberculosis, Bordetella pertussis, Haemophilus influenzae, Moraxella catarrhalis, Pseudomonas aeruginosa, Stenotrophomonas maltophila, Staphylococcus aureus, Streptococcus pyogenes, Neisseria meningitidis, Klebsiella pneumoniae, or Non-tuberculosis Mycobacterium.
Clause 46. The method of clause 43, wherein the parasitic infection is malaria.
Clause 47. The method of any one of clauses 40-46, wherein the one or more tryptamines are psilocybin, psilocin, norpsilocin, baeocystin, norbaeocystin, N,N-dimethyltryptamine (DMT), or combinations thereof.
Clause 48. The method of any one of clauses 40-47, wherein the monoamine oxidase inhibitor is Norharman, Harmine, 1,2,3,4-tetrahydro-β-carboline-3-carboxylic acid, 1-methyl-1,2,3,4-tetrahydro-β-carboline-3-carboxylic acid, 1-methyl-2,3,4,9-tetrahydro-1H-β-carboline-1,3-dicarboxylic acid, Harmaline, N-methoxy-1-vinyl-β-carboline, ethyl 9H-β-arboline-3-carboxylate, 1-furyl-β-carboline-3-carboxylic acid, 1-[5-(methoxymethyl)-2-furyl]-9H-β-carboline-3-carboxylic acid, 6-hydroxy-3-(6-hydroxy-1H-indol-3-yl)-9H-β-carboline-4-carboxylic acid, Strictosidine, (1S)-1-{[(2S,3R,4S)-2-(β-L-glucopyranosyloxy)-5-(methoxycarbonyl)-3-vinyl-3,4-dihydro-2H-pyran-4-yl]methyl}-2,3,4,9-tetrahydro-1H-β-carboline-1,3-dicarboxylic acid, or combinations thereof.
Clause 49. The method of any one of clauses 40-48, wherein inflammation is reduced and neuroregeneration is induced in the subject.
Clause 50. The method of clause 49, wherein neuroregeneration comprises neurite outgrowth.
Clause 51. The method of any one of clauses 14-28, wherein the infectious disease or condition causes neurological damage in the subject and the method results in treatment of the neurological damage.
Clause 52. The method of any one of clauses 40-50, wherein the infectious disease or condition causes neurological damage in the subject and the method results in treatment of the neurological damage.

### EXAMPLES

### Example 1

### The impact of Hericium erinaceus (He, Lion's Mane Mushroom Mycelium) and Tryptamine/Psilocybin Analogs on IL-10 Protein Expression

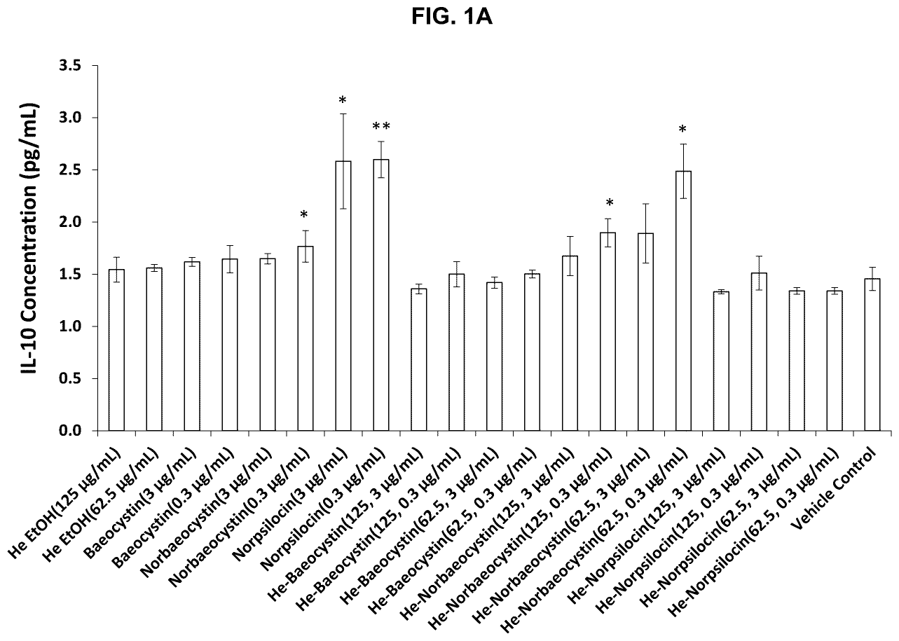

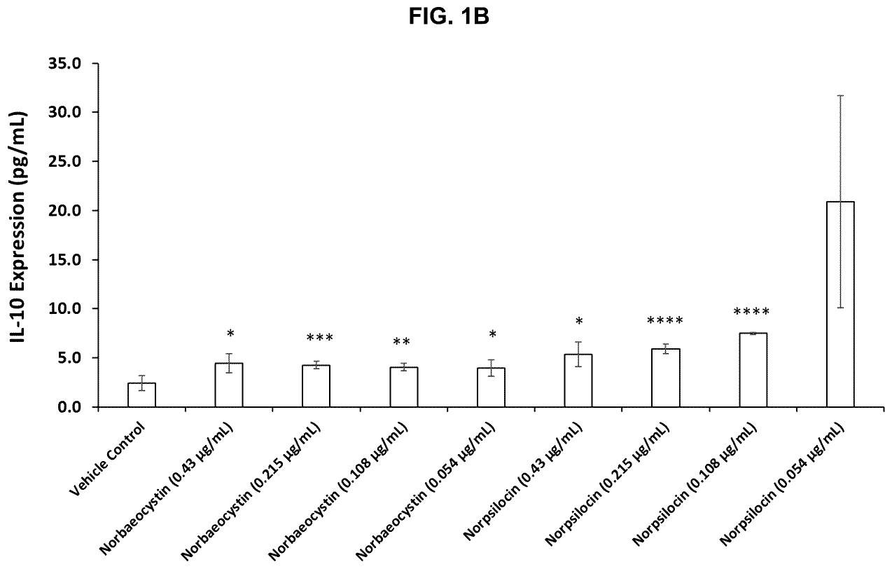

Treatment of 1321N1 human brain cells with norpsilocin (3 μg/mL; 0.3 μg/mL), norbaeocystin (0.3 μg/mL), and He-norbaeocystin (125, 0.3 μg/mL; 62.5, 0.3 μg/mL) was found to significantly upregulate the expression of the anti-inflammatory cytokine IL-10 via ELISA (FIG. 1A). It was both surprising and unexpected to find that ethyl acetate extracts of He, combined with norbaeocystin, increase IL-10 expression in these brain cells. These data and results demonstrate an anti-inflammatory effect for certain tryptamine/psilocybin components and analogs both individually and in combination formulations with He or PEP, and Niacin.

In a separate experiment, human peripheral blood mononuclear cells (PBMCs) were treated with different concentrations of norbaeocystin, norpsilocin, and vehicle control under standard non-inflammatory conditions, and IL-10 expression was measured. The results shown in FIG. 1B demonstrate that these individual tryptamine/psilocybin analogs significantly upregulate the expression of the anti-inflammatory cytokine IL-10 at multiple concentrations. Additionally, combination formulations comprising He-Psilocin-Niacin or PEP-Psilocin-Niacin resulted in very minimal pro-inflammatory responses.

### Example 2

### β-carbolines in He Mycelium

It was identified that several β-carbolines are present in extracts of He mycelium cultivated on rice, suggesting that He mycelium grown on grains contains the constituents listed below in Table 6. Notably, it was surprising and unexpected to detect harmine, a reversible inhibitor of MAO-A (RIMA), in ethyl acetate extracts of He mycelium. At least one of the compounds in Table 6, 2,3,4,9-Tetrahydro-1H-β-carboline-3-carboxylic acid, has been detected in He fruitbodies (Yang et al., J. Food Quality 2021: 560626 (2021)). Yang et al. did not detect the compound harmine.

TABLE 6
Beta-carbolines and related molecules putatively detected in extracts
of Hericium erinaceus mycelium using mass spectroscopy.

| Compound Name | Formula | Mass (Da) | Certainty* | Ions |
|---|---|---|---|---|
| Norharman | C₁₁H₈N₂ | 168.07 | High | +/− |
| Harmine | C₁₃H₁₂N₂O | 212.10 | High | +/− |
| 1,2,3,4-tetrahydro-beta-carboline-3- | C₁₂H₁₂N₂O₂ | 216.09 | High | +/− |
| carboxylic acid | | | | |
| 1-methyl-1,2,3,4-tetrahydro-beta- | C₁₃H₁₄N₂O₂ | 230.11 | High | +/− |
| carboline-3-carboxylic acid | | | | |
| 1-methyl-2,3,4,9-tetrahydro-1H-beta- | C₁₄H₁₄N₂O₄ | 274.10 | High | +/− |
| carboline-1,3-dicarboxylic acid | | | | |
| Harmaline | C₁₃H₁₄N₂O | 214.11 | Medium | + |
| N-methoxy-1-vinyl-beta-carboline | C₁₄H₁₂N₂O | 224.10 | Medium | + |
| Ethyl 9H-beta-carboline-3-carboxylate | C₁₄H₁₂N₂O₂ | 240.09 | Medium | + |
| 1-furyl-beta-carboline-3-carboxylic acid | C₁₆H₁₀N₂O₃ | 278.07 | Medium | − |
| 1-[5-(methoxymethyl)-2-furyl]-9H-beta- | C₁₃H₁₄N₂O₄ | 322.09 | Medium | − |
| carboline-3-carboxylic acid | | | | |
| 6-hydroxy-3-(6-hydroxy-1H-indol-3-yl)- | C₂₀H₁₃N₃O₄ | 359.09 | Low | − |
| 9H-beta-carboline-4-carboxylic acid | | | | |
| Strictosidine | C₂₇H₃₄N₂O₉ | 530.23 | Low | − |
| (1S)-1-{[(2S,3R,4S)-2-(beta-L- | C₂₃H₃₄N₂O₁₁ | 574.22 | Low | − |
| glucopyranosyloxy)-5- | | | | |
| (methoxycarbonyl)-3-vinyl-3,4-dihydro- | | | | |
| 2H-pyran-4-yl]methyl}-2,3,4,9- | | | | |
| tetrahydro-1H-beta-carboline-1,3- | | | | |
| dicarboxylic acid | | | | |

*Certainty is described as High if both positive and negative ions featured matching retention times, Medium if two or more annotation databases showed matches, and Low if only the compound was annotated using a single database.

### Example 3

Future studies will focus on confirming the detection of compounds like harmine in He mycelium using mass spectroscopy with reference standards of known analytes. Future studies will also aim to clarify the role of MAOIs in influencing blood-brain barrier (BBB) permeability of tryptamines in combination with He. The human temporal lobe microvessel cell line hCMEC/D3 will be utilized, which serves as a well-established BBB model (Weksler et al., Fluids Barriers CNS, 10(1): 16 (2013)). The hCMEC/D3 cells will be cultured and passaged into an in vitro BBB transwell system such that cells are cultured on a microporous membrane transwell insert. Compounds will be added to the upper compartment to determine if they pass through the microvessel cells into the lower compartment, providing insight into BBB permeability (Bikhezar et al., J. Neurooncology 146(2): 239-246 (2020)). The in vitro BBB transwell system will be tested for the ability of tryptamine and He components to permeate the BBB in the presence and absence of MAOIs. Moreover, cells may be harvested to determine the impact of tryptamine and He components in combination with MAOIs on the expression of genes and proteins of interest, such as serotonin specific transporters (SERTs), cytochrome p450s, and cytokines, which influence BBB permeability.

Extracts of He mycelium on grain that contains endogenous MAOIs (e.g., harmine) may improve the pharmacokinetic profile of tryptamines such as norpsilocin, baeocystin, and norbaeocystin through either delayed degradation and/or improved passage through the BBB. Clinical benefits of the combinations of tryptamines with He mycelium and MAOIs may increase upregulation of anti-inflammatory IL-10 proteins, which are helpful for neurological and immune health. The benefits of formulating compositions with MAOIs may also extend to other tryptamines and psilocybin analogs, such as aeruginascin.

### Example 4

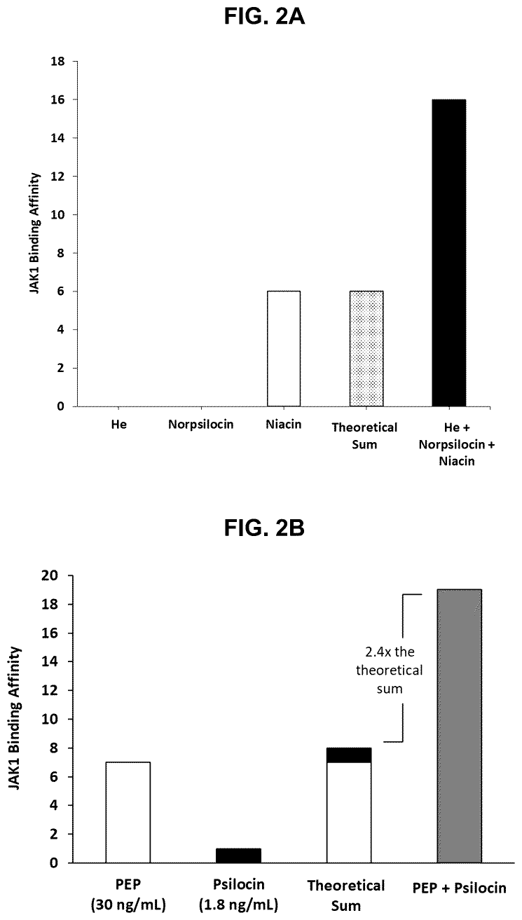

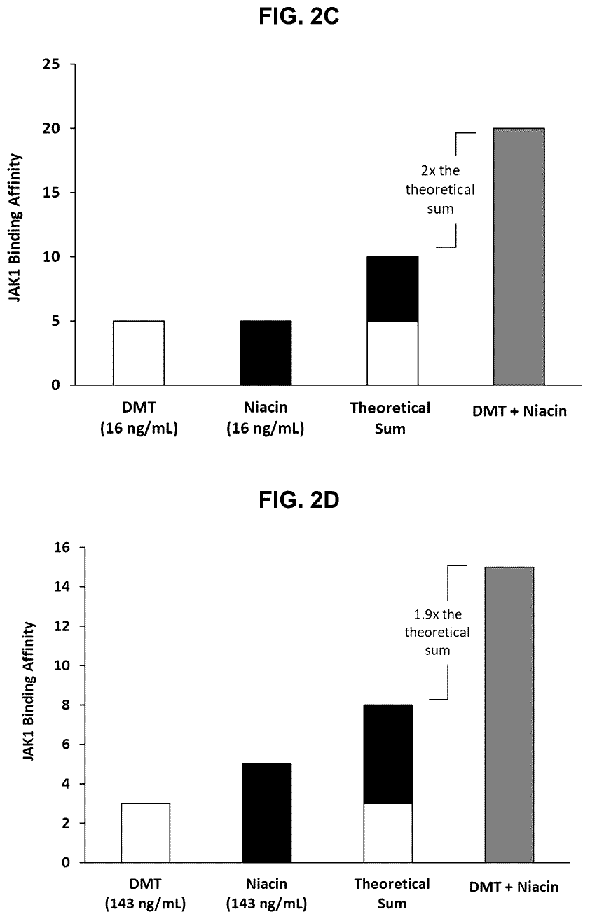

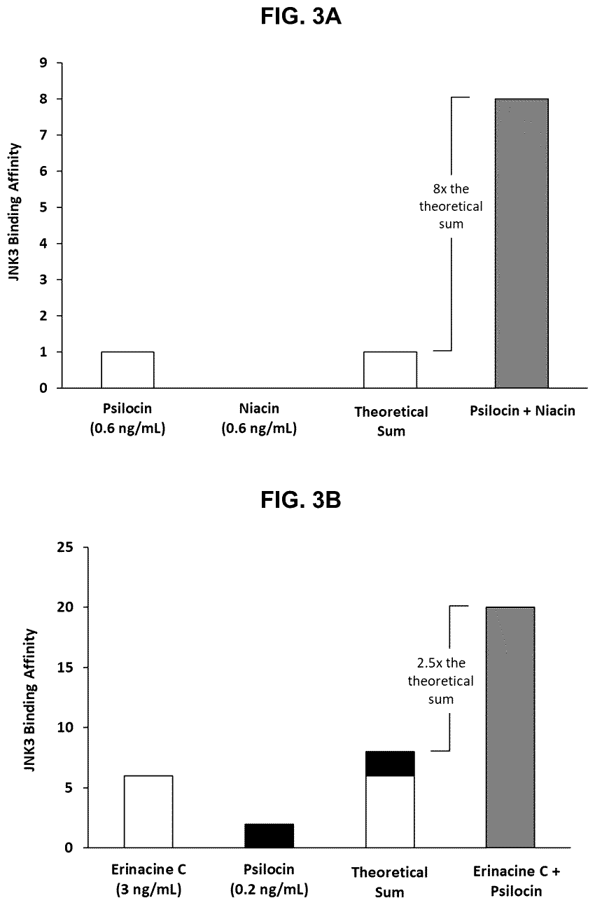

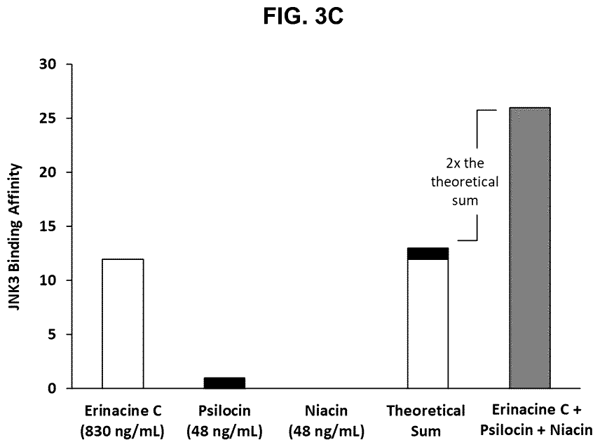

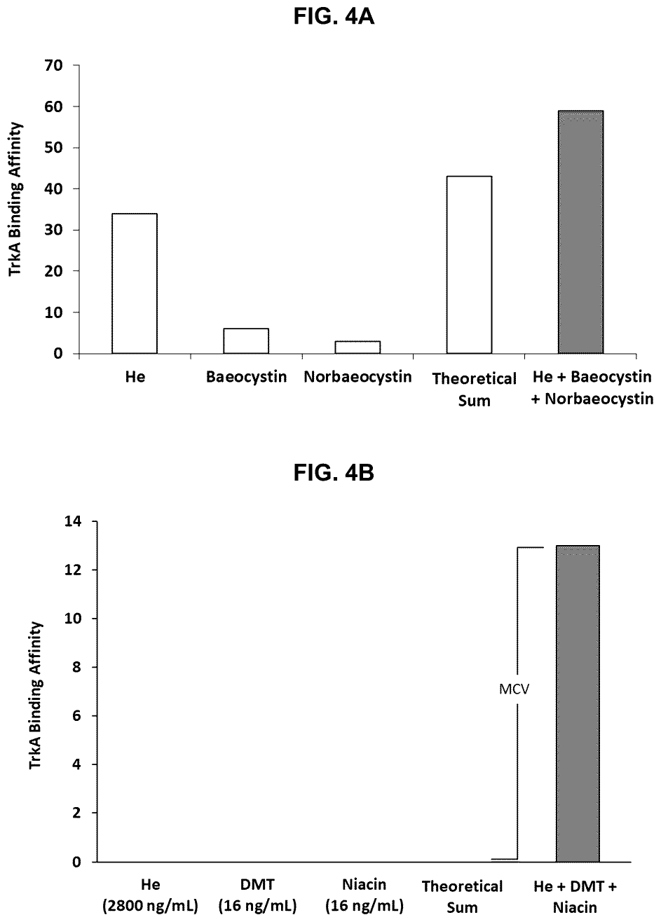

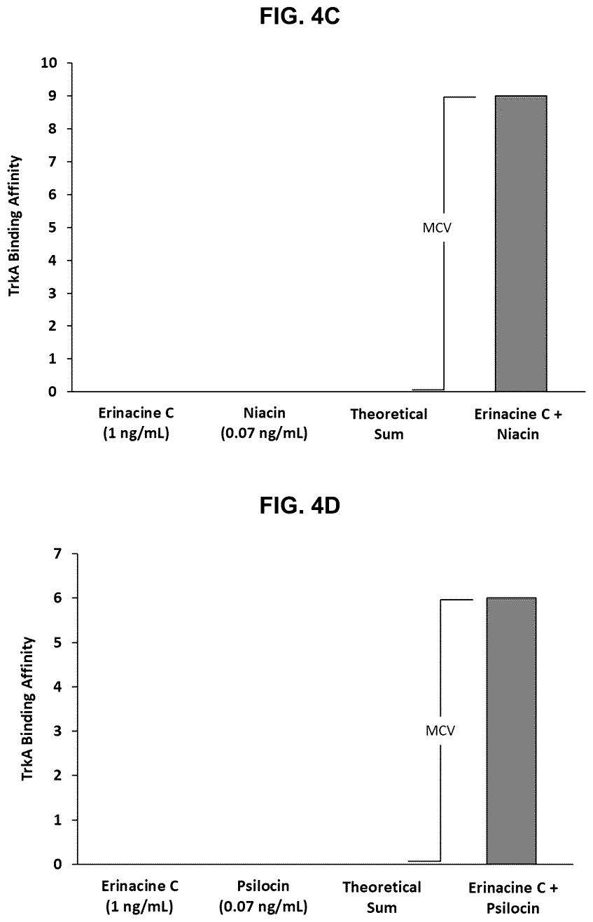

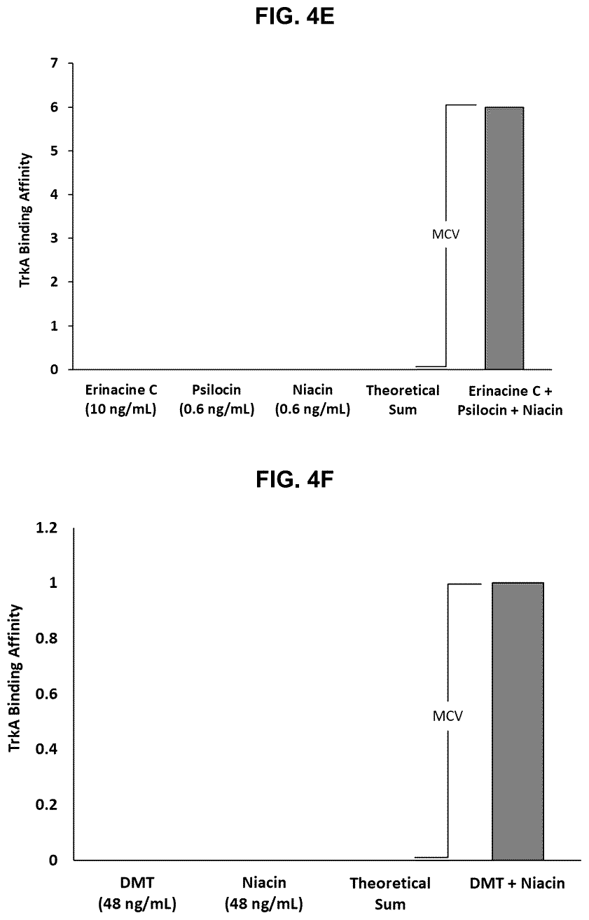

It was identified that many combination formulations of tryptamines, He/PEP/Erinacine C, and niacin can interact with several mitogen-activated protein kinases (MAPKs) involved with brain health and can even provide synergistic benefits (Tables 7-8). Surprisingly, niacin was found to provide a synergistic effect on a neuroinflammation MAPK regulatory protein, Janus kinase 1 (JAK1) that is required for production of the anti-inflammatory cytokine IL-10, when combined with He and norpsilocin (FIG. 2A). Various other combination formulations also resulted in synergistic effects on JAK1 binding affinity (FIG. 2B—D), as well as the binding affinity for other MAPKs involved in anti-inflammatory pathways, including c-Jun N-terminal Kinase 3 (JNK3) (FIG. 3A-C) and Tropomyosin Receptor Kinase A (TRKA) (FIG. 4A-F), which is a MAPK associated with the NGF receptor, stimulating neurogenesis throughout the CNS.

It was surprising and unexpected that such dilute psilocin at low ng concentrations produced a synergistic effect on MAPK binding when combined with other components because these low concentrations had no effect on MAPK binding when used alone. Therefore, one would have expected these low concentrations to be ineffective. The low concentrations also overcome the pharmacodynamic issues faced when using high concentrations of psilocin.

One of the top hits revealed in the MAPK screen was JNK3 with the He-Agarikon (Fomitopsis officinalis, Fo) formulation (Table 7). Although the literature has suggested a role of He in influencing JNK, it appears that there may be disparate mechanisms by which He promotes the activation or inhibition of this MAPK, as it has been previously identified to be activated by He in 1321N1 human microglial cells, while it has been inhibited in murine colorectal carcinoma CT-26 cells (Mori et al., Biolog. Pharmaceut. Bul. 31(9): 1727-1732 (2008); Kim et al., J. Agri. Food Chem. 61(20): 4898-4904 (2013)). Future research will aim to utilize MAPK assays to reveal potential bioactivity of psilocin and psilocybin individually and when combined with He and/or niacin, and aim to utilize an alternative, more immunologically active cell model to potentially provide more robust neuroinflammatory responses. Cytokine and MAPK ELISAs in murine BV2 microglial cells will be explored to better elucidate the potential synergistic effects of tryptamines with He and/or niacin.

TABLE 7
Synergistic Effects of Tryptamine and He Combination Formulations Through Interaction
with MAPKs Involved with Neuronal Health (MARK Binding Affinity Values).

| | | | | | | | | | | | Theoretical | |
|---|---|---|---|---|---|---|---|---|---|---|---|---|
| Tested | | | Entrez | | | | | | | | Sum of | |
| Combination | Concentration | DiscoveRx | Gene | Percent | | | | | | | Combination | Fold |
| Formulation | (μg/mL) | Gene Symbol | Symbol | Control | HE | Baeo | Norbaeo | Norpsilo | FO | Niacin | Formulation | Change |
| HE-Norpsilo | 250 | p38-alpha | MAPK14 | 68 | 5 | — | — | 21 | — | — | 26 | 6 |
| HE-Baeo- | 125 | JNK3 | MAPK10 | 44 | 38 | 8 | 4 | — | — | — | 50 | 6 |
| Norbaeo | | | | | | | | | | | | |
| HE-Baeo | 125 | TRKA | NTRK1 | 34 | 56 | 3 | — | — | — | — | 59 | 7 |
| Baeo-Norbaeo | 10 | p38-alpha | MAPK14 | 75 | — | 1 | 17 | — | — | — | 18 | 7 |
| Baeo-Norbaeo- | 5 | TRKB | NTRK2 | 81 | — | 12 | 0 | 0 | — | — | 12 | 7 |
| Norpsilo | | | | | | | | | | | | |
| HE-Norbaeo | 62.5 | JAK1 | JAK1 | 89 | 0 | — | 3 | — | — | — | 3 | 8 |
| | | (JH1domain- | | | | | | | | | | |
| | | catalytic) | | | | | | | | | | |
| HE-Baeo- | 62.5 | ROCK2 | ROCK2 | 81 | 8 | 0 | — | 3 | — | — | 11 | 8 |
| Norpsilo | | | | | | | | | | | | |
| HE-Niacin- | 250 | JAK1 | JAK1 | 84 | 0 | — | — | 0 | — | 6 | 6 | 10 |
| | | (JH1domain- | | | | | | | | | | |
| Norpsilo | | catalytic) | | | | | | | | | | |
| HE-Baeo | 250 | p38-alpha | MAPK14 | 82 | 5 | 1 | — | — | — | — | 6 | 12 |
| HE-Baeo | 250 | TRKA | NTRK1 | 48 | 34 | 6 | — | — | — | — | 40 | 12 |
| Baeo-Norbaeo- | 2.5 | p38-alpha | MAPK14 | 49 | — | 2 | 22 | 15 | — | — | 39 | 12 |
| Norpsilo | | | | | | | | | | | | |
| HE/FO EtOAc | 62.5 | JAK1 | JAK1 | 86 | 0 | — | — | — | 0 | — | 0 | 14 |
| | | (JH1domain- | | | | | | | | | | |
| | | catalytic) | | | | | | | | | | |
| Baeo-Norbaeo | 2.5 | JAK1 | JAK1 | 82 | — | 1 | 3 | — | — | — | 4 | 14 |
| | | (JH1domain- | | | | | | | | | | |
| | | catalytic) | | | | | | | | | | |
| HE-Baeo- | 250 | p38-alpha | MAPK14 | 58 | 5 | 1 | — | 21 | — | — | 27 | 15 |
| Norpsilo | | | | | | | | | | | | |
| HE-Baeo- | 250 | TRKA | NTRK1 | 41 | 34 | 6 | 3 | — | — | — | 43 | 16 |
| Norbaeo | | | | | | | | | | | | |
| Baeo-Norpsilo | 5 | p38-alpha | MAPK14 | 73 | — | 9 | — | 1 | — | — | 10 | 17 |
| Baeo-Norbaeo- | 10 | p38-alpha | MAPK14 | 44 | — | 1 | 17 | 21 | — | — | 39 | 17 |
| Norpsilo | | | | | | | | | | | | |
| Norbaeo- | 5 | JNK3 | MAPK10 | 58 | — | — | 4 | 19 | — | — | 23 | 19 |
| Norpsilo | | | | | | | | | | | | |
| HE-Norbaeo | 250 | TRKA | NTRK1 | 41 | 34 | — | 3 | — | — | — | 37 | 22 |
| Baeo-Norpsilo | 10 | p38-alpha | MAPK14 | 51 | — | 1 | — | 21 | — | — | 22 | 27 |
| Baeo-Norbaeo- | 5 | p38-alpha | MAPK14 | 50 | — | 9 | 12 | 1 | — | — | 22 | 28 |
| Norpsilo | | | | | | | | | | | | |
| Norbaeo- | 10 | p38-alpha | MAPK14 | 29 | — | — | 17 | 21 | — | — | 38 | 33 |
| Norpsilo | | | | | | | | | | | | |
| Norbaeo- | 2.5 | p38-alpha | MAPK14 | 25 | — | — | 22 | 15 | — | — | 37 | 38 |
| Norpsilo | | | | | | | | | | | | |
| Norbaeo- | 5 | p38-alpha | MAPK14 | 12 | — | — | 12 | 1 | — | — | 13 | 75 |
| Norpsilo | | | | | | | | | | | | |

TABLE 8
Synergistic Effects of Tryptamine, Erinacine C, PEP, and Niacin Combination Formulations Through
Interaction with MAPKs Involved with Neuronal Health (MARK Binding Affinity Values).

| | | | | | | | | | | Theoretical | Measured | |
|---|---|---|---|---|---|---|---|---|---|---|---|---|
| Tested | | Entrez | | | | | | | | Sum of | Combination | |
| Combination | DiscoveRx | Gene | Concentration | Percent | | Erinacine | | | | Combination | Formulation | Fold |
| Formulation | Gene Symbol | Symbol | (ng/mL) | Control | PEP | C | Psilocin | DMT | Niacin | Formulation | Value | Change |
| Psilocin- | JNK3 | MAPK10 | 0.6, 0.6 | 92 | — | — | 1 | — | 0 | 1 | 8 | 8 |
| Niacin | | | | | | | | | | | | |
| Erinacine C- | TRKA | NTRK1 | 1, 0.07 | 91 | — | 0 | — | — | 0 | 0 | 9 | MCV* |
| Niacin | | | | | | | | | | | | |
| Erinacine C- | TRKA | NTRK1 | 1, 0.07 | 94 | — | 0 | 0 | — | — | 0 | 6 | MCV* |
| Psilocin | | | | | | | | | | | | |
| PEP-Psilocin | TRKA | NTRK1 | 1, 0.07 | 97 | 0 | — | 0 | — | — | 0 | 3 | MCV* |
| PEP-Psilocin- | TRKA | NTRK1 | 1, 0.07, | 97 | 0 | — | 0 | — | 0 | 0 | 3 | MCV* |
| Niacin | | | 0.07 | | | | | | | | | |
| Psilocin- | TRKA | NTRK1 | 1.8, 1.8 | 97 | — | — | 1 | — | 0 | 1 | 3 | 3 |
| Niacin | | | | | | | | | | | | |
| PEP-Psilocin | TRKA | NTRK1 | 3, 0 2 | 99 | 0 | — | 0 | — | — | 0 | 1 | MCV* |
| Erinacine C- | JNK3 | MAPK10 | 3, 0.2 | 80 | — | 6 | 2 | — | — | 8 | 20 | 2.5 |
| Psilocin | | | | | | | | | | | | |
| PEP-Psilocin- | TRKA | NTRK1 | 3, 0.2, | 99 | 0 | — | 0 | — | 0 | 0 | 1 | MCV* |
| Niacin | | | 0.2 | | | | | | | | | |
| Erinacine C- | TRKA | NTRK1 | 10, 0.6 | 97 | — | 0 | 0 | — | — | 0 | 3 | MCV* |
| Psilocin | | | | | | | | | | | | |
| PEP-Psilocin | TRKA | NTRK1 | 10, 0.6 | 99 | 0 | — | 0 | — | — | 0 | 1 | MCV* |
| Erinacine C- | TRKA | NTRK1 | 10, 0.6, | 94 | — | 0 | 0 | — | 0 | 0 | 6 | MCV* |
| Psilocin- | | | 0.6 | | | | | | | | | |
| Niacin | | | | | | | | | | | | |
| PEP-Psilocin- | TRKA | NTRK1 | 10, 0.6, | 96 | 0 | — | 0 | — | 0 | 0 | 4 | MCV* |
| Niacin | | | 0.6 | | | | | | | | | |
| DMT-Niacin | JAK1 | JAK1 | 16, 16 | 80 | — | — | — | 5 | 5 | 10 | 20 | 2 |
| | (JH1domain- | | | | | | | | | | | |
| | catalytic) | | | | | | | | | | | |
| PEP-Psilocin | JAK1 | JAK1 | 30, 1.8 | 81 | 7 | — | 1 | — | — | 8 | 19 | 2.375 |
| | (JH1domain- | | | | | | | | | | | |
| | catalytic) | | | | | | | | | | | |
| Erinacine C- | TRKA | NTRK1 | 30, 1.8, | 88 | — | 0 | 1 | — | 0 | 1 | 12 | 12 |
| Psilocin- | | | 1.8 | | | | | | | | | |
| Niacin | | | | | | | | | | | | |
| DMT-Niacin | TRKA | NTRK1 | 48, 48 | 99 | — | — | — | 0 | 0 | 0 | 1 | MCV* |
| Erinacine C- | TRKA | NTRK1 | 90, 5.3 | 95 | — | 0 | 0 | — | — | 0 | 5 | MCV* |
| Psilocin | | | | | | | | | | | | |
| PEP-Psilocin | TRKA | NTRK1 | 90, 5.3 | 96 | 0 | — | 0 | — | — | 0 | 4 | MCV* |

MCV*-Maximum Calculable Value: refers to an instance in which a combination formulation provides binding affinity, while the individual components do not

### Example 5

### The Impact of He, PEP, Psilocin, and Niacin Formulations on Neurite Outgrowth in Rat PC12 Cells

Rat PC12 cells are a cell line derived from rat pheochromocytoma and are an immortalised cell line similar to the primary culture of fetal neurons. These cells are relatively easy to passage and culture and have some features of neurons, which makes them useful in the study of nerve physiology and pharmacology. PC12 cells were treated with He Myc EtOAc (3.5-28 μg/mL), Putative Erinacine Peak (PEP) (3.125-25 μg/mL), psilocin (14-430 ng/mL), niacin (0.175-1.4 μg/mL), a He-Psilocin-Niacin formulation, a PEP-Psilocin-Niacin formulation, vehicle (DMSO) negative control, or nerve growth factor (NGF) positive control and incubated for 6 days before neurite outgrowth was evaluated using microscopic imaging analysis.

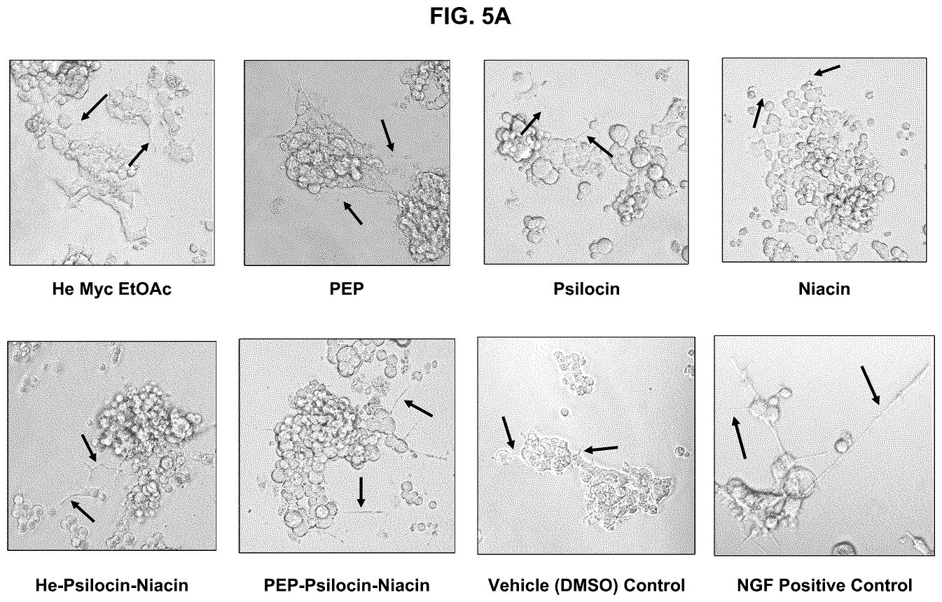

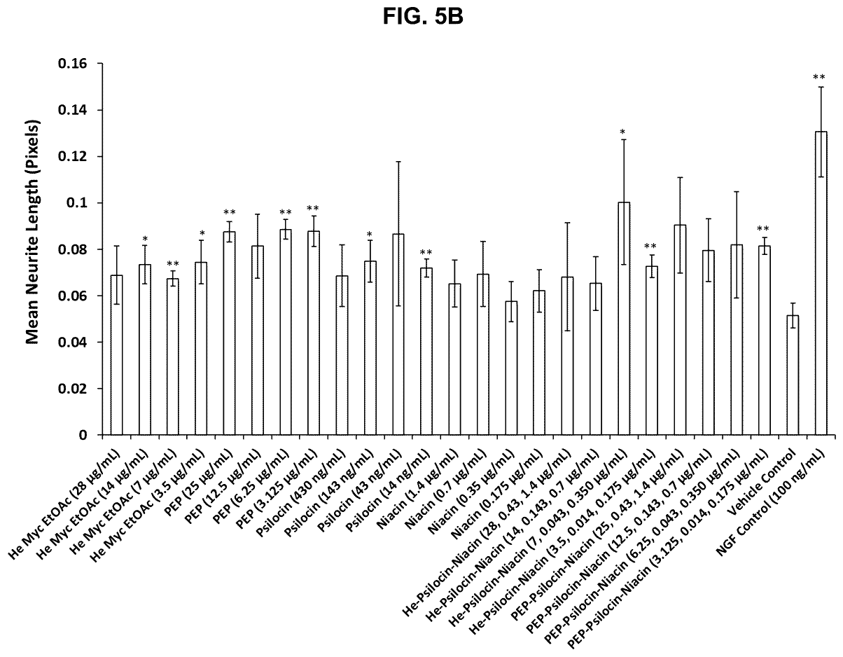

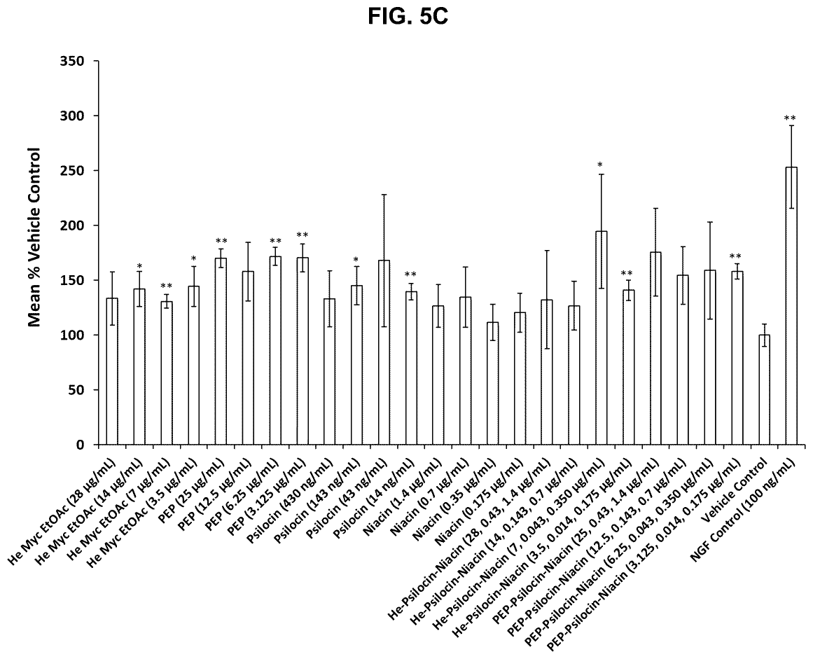

FIG. 5A shows representative images of the PC12 cells treated with He Myc EtOAc (7 μg/mL), PEP (6.25 μg/mL), psilocin (0.043 μg/mL), niacin (0.35 μg/mL), a He-Psilocin-Niacin formulation (7, 0.043, 0.35 μg/mL), a PEP-Psilocin-Niacin formulation (7, 0.043, 0.35 μg/mL), vehicle (DMSO) negative control, and NGF positive control. The arrows in FIG. 5A indicate regions of neurite outgrowth. FIG. 5B shows a graph of the mean neurite length (pixels) for each of the treatments. FIG. 5C shows the mean neurite growth for each treatment as a relative % of vehicle control. The highest mean outgrowth was observed with He-psilocin-niacin treatment (7, 0.043, 0.35 μg/mL), where a 194% increase was observed relative to the vehicle (DMSO) control. In comparison, the highest mean outgrowth for He treatment was 131% of vehicle control (7 μg/mL), the highest mean outgrowth for psilocin treatment was 168% of vehicle control (0.043 μg/mL), and the highest mean outgrowth for niacin treatment was 112% of vehicle control (0.35 μg/m L).

These results are very significant because cultured cell lines grow radially. Therefore, a 6-day increase from 168% with psilocin to 194% with the He-psilocin-niacin formulation has a dramatic long-term effect. The increase in neurite outgrowth becomes amplified as neurites continue to grow, bifurcate in different directions, etc., making this effect more exponential than linear. This impact on neurite outgrowth and lengthening may be due in part to the effects that the combination formulations have on IL-10 expression levels and MAPK binding that is involved in neuronal health, as described in Examples 1 and 4, respectively. Interestingly, previous studies have found that IL-10 not only attenuates neuronal apoptosis, but also promotes neurite outgrowth and synapse formation via the JAK1/STAT3 signaling pathway in cultured primary cortical neurons after OGD injury (Chen et al., Scientific Reports 6(1): 30459 (2016)). Although IL-10 has been shown to promote neurite outgrowth, it was surprising to observe neurite outgrowth as shown in FIG. 5. This is because it is known that inflammation is also required for induction of neurite outgrowth (e.g., Pitake et al., Front. Neurosci., 13: (2019)), but the compositions as described herein induce an anti-inflammatory response and reduce the inflammatory response. Therefore, the compositions as described herein provide both neuroregenerative and anti-inflammatory effects and may prevent neurotoxic effects associated with inflammation during neuroregeneration and cell division.

### Example 6

### The Impact of He, Psilocin, and Niacin Formulations on the Expression of IL-10, IL-1RA, and Phospho-JNK in Murine BV2 Microglial Cells

Future studies will further investigate whether formulations of psilocin, He, and niacin promote the expression of IL-10 and IL-1RA, influence the activation of phospho-JNK, and provide broad impacts on MAPK signaling.

### Materials and Reagents

BV2 cells; culture media; trypsin; PBS; flasks; 96 well plates; ELISA kits including: IL-10, IL-1RA, phospho-JNK; JNK inhibitor; qRT-PCR primers.

### Methods

Murine BV2 microglial cells will be cultured and grown to confluency in T75 flasks. Subsequently, cells will be trypsinized and seeded into several 96 well plates, grown to confluency, starved for 24 hours, and treated with individual compounds and formulations. Treatments include an He ethyl acetate (EtOAc) fraction, a column-derived putative erinacine peak (PEP) fraction that putatively contains a concentrated erinacine pool, psilocin, and nicotinic acid (B3). An IL-10 ELISA will be conducted to compare the anti-inflammatory effects of psilocin versus the psilocybin analogs of baeocystin, norbaeocystin, and norpsilocin. The concentration ranges to be tested for the ELISA assays are illustrated below in Table 9. 200 pL of conditioned media from each well will be harvested for ELISA assays. Assays will focus on the impact of psilocin, He, niacin, and permutations on the expression of IL-1RA, IL-10, and phospho-JNK protein. Subsequent ELISAs may be conducted to corroborate findings and/or explore other proteins of interest, such as IL-4 and IL-6. The ELISAs assays will be run mostly in parallel. The data from the ELISAs may reveal optimal conditions for synergy, which could be applied to subsequent qRT-PCR work to profile the expression of a broader range of related gene targets, including growth factors, MAPKs, and cytokines.

TABLE 9
Concentrations and Equivalent Human Doses for ELISA Assays

| | | | |
|---|---|---|---|
| Concentration 1 | Concentration 2 | Concentration 3 | Concentration 4 |

Assay Concentration

| | | | | | | | | |
|---|---|---|---|---|---|---|---|---|
| Psilocin | 430 | ng/mL | 143 | ng/mL | 43 | ng/mL | 14 | ng/mL |
| Niacin | 1.4 | μg/mL | 0.7 | μg/mL | 0.35 | μg/mL | 0.175 | μg/mL |
| He | 28 | μg/mL | 14 | μg/mL | 7 | μg/mL | 3.5 | μg/mL |
| PEP | 25 | μg/mL | 12.5 | μg/mL | 6.25 | μg/mL | 3.125 | μg/mL |

Human Equivalent Dose (mg/70 kg)

| | | | | |
|---|---|---|---|---|
| Psilocin | 30 | 10 | 3 | 1 |
| Niacin | 100 | 50 | 25 | 12.5 |
| He | 1960 | 980 | 490 | 245 |
| PEP | 1750 | 875 | 437.5 | 218.75 |

Future studies will also aim to investigate if niacin, psilocin, and He possess complementary mechanisms by which they influence anti-inflammatory activity and MAPK signaling, which may provide a synergistic effect. While it is possible that synergistic effects may not be observed, these data could still serve to substantiate efficacy of the combination of niacin, psilocin, and He, and also demonstrate a mechanism by which He promotes anti-neuroinflammatory activity.

### Example 7

### MAPK Kinome Profiling

Future studies will further investigate the MAPK kinome profile of sample sets including He and PEP combined with psilocin and niacin, as shown below in Table 10. Turkey tail (Trametes versicolor, Tv) will also be included to provide preliminary data in support of interest in its potential inclusion in formulations. The specific MAPK kinome profile targets are listed below in Table 11.

TABLE 10
MARK Profiling Screen: Human Dose Equivalencies

| | Concentrations | | | Human Equivalent Dose | | | | | |
|---|---|---|---|---|---|---|---|---|---|
| Compound | (μg/mL) | | | (Assuming 70 kg human) | | | | | |
| He | 250 | 125 | 62.5 | 17.5 | g | 8.75 | g | 4.375 | g |
| PEP | 50 | 25 | 12.5 | 3.5 | g | 1.75 | g | 0.875 | g |
| Tv | 250 | 125 | 62.5 | 17.5 | g | 8.75 | g | 4.375 | g |
| Psilocybin | 10 | 5 | 2.5 | 700 | mg | 350 | mg | 175 | mg |
| Psilocin | 10 | 5 | 2.5 | 700 | mg | 350 | mg | 175 | mg |
| Niacin | 5 | 2.5 | 1.25 | 350 | mg | 175 | mg | 87.5 | mg |

TABLE 11
MARK Kinome Profile Targets

| | DiscoveRx | Entrez Gene | |
|---|---|---|---|
| MARK | Gene Symbol | Symbol | Description |
| Janus Kinase 1 | JAK1 | JAK1 | Cytokine signaling, including IL- |
| | (JH1domain- | | 2, IL-4, IFN-α/β, IFN-γ, IL-10 |
| | catalytic) | | |
| c-Jun N-Terminal Kinase 3 | JNK3 | MAPK10 | Neuronal specific protein |
| | | | isoform; regulation of apoptosis |
| p38-α | p38-alpha | MAPK14 | Immune cell macrophage |
| | | | activation marker; TNF-α |
| | | | induction |
| Rho Associated Coiled-Coil | ROCK2 | ROCK2 | Neurite retraction, inflammation |
| Containing Protein Kinase 2 | | | |
| Tropomyosin Receptor | TRKA | NTRK1 | NGF receptor, stimulating |
| Kinase A | | | neurogenesis throughout the |
| | | | CNS |
| Tropomyosin Receptor | TRKB | NTRK2 | BDNF receptor |
| Kinase B | | | |
| Tropomyosin Receptor | TRKC | NTRK3 | NT-3 receptor |
| Kinase C | | | |
| Extracellular Signal- | ERK2 | MAPK1 | Inflammation, myelination, |
| Regulated Kinase 2 | | | neuroinflammatory disease |
| | | | response |
| Interleukin-1 Receptor- | IRAK1 | IRAK1 | Inflammation, ROS response, |
| Associated Kinase 1 | | | Antioxidant Activity |

### Example 8

### The Impact of Different Individual Components and Combination Formulations on IL-6 Expression in Human PBMCs

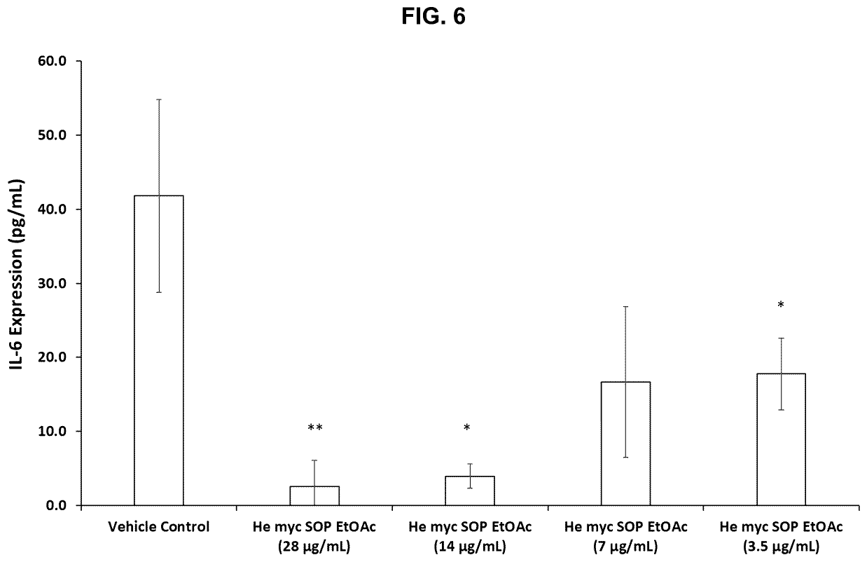

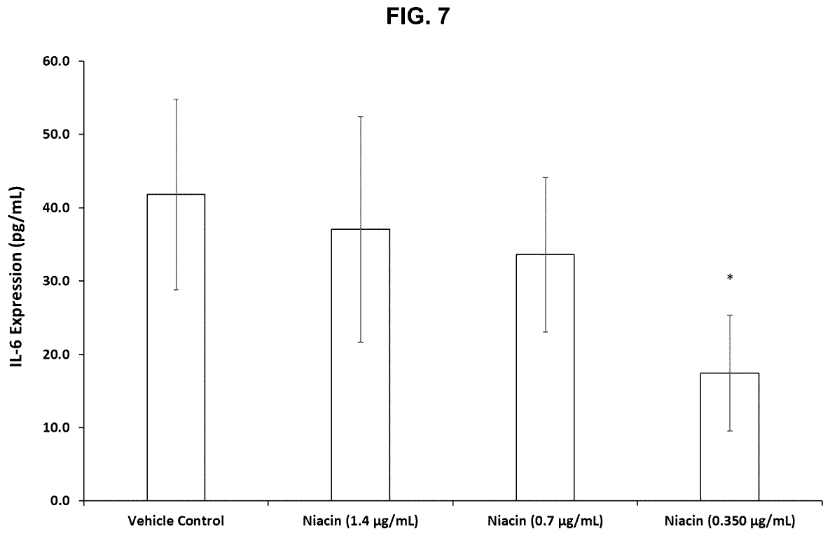

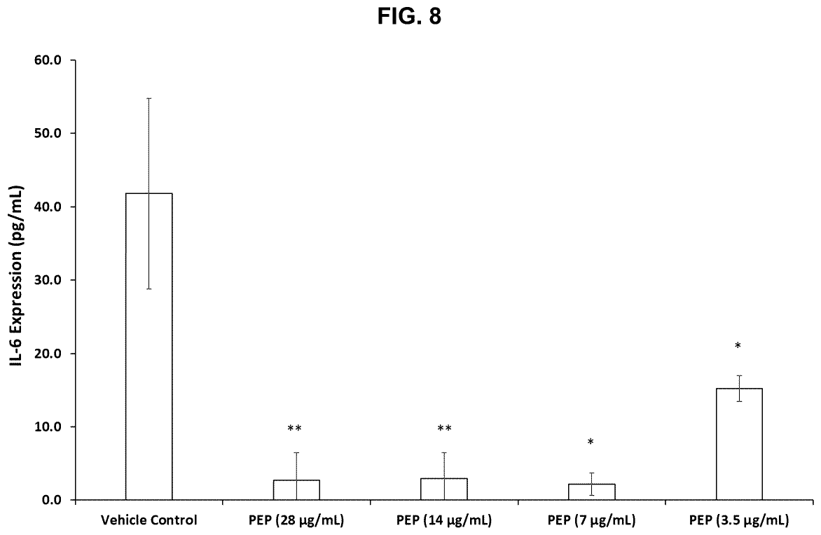

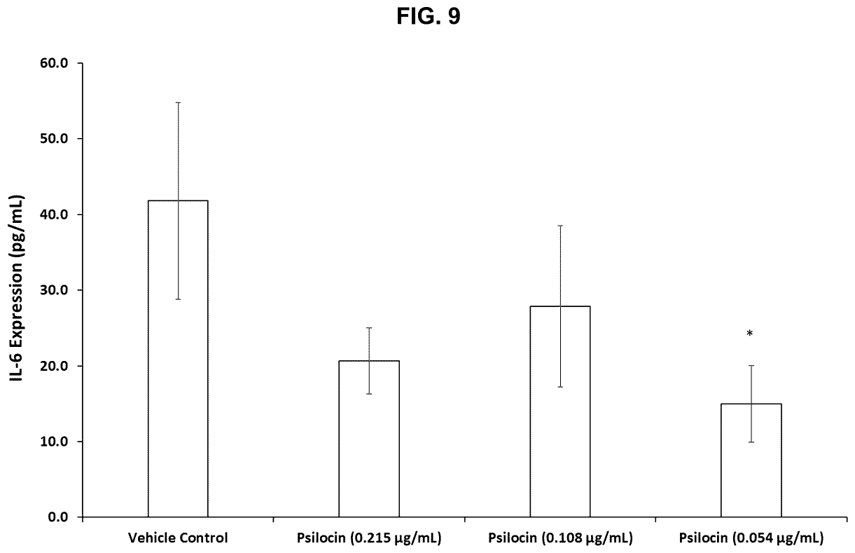

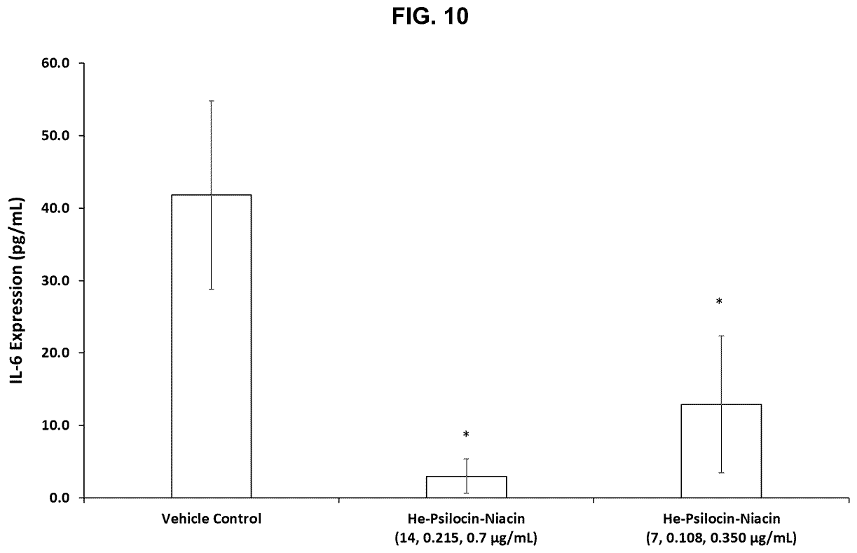

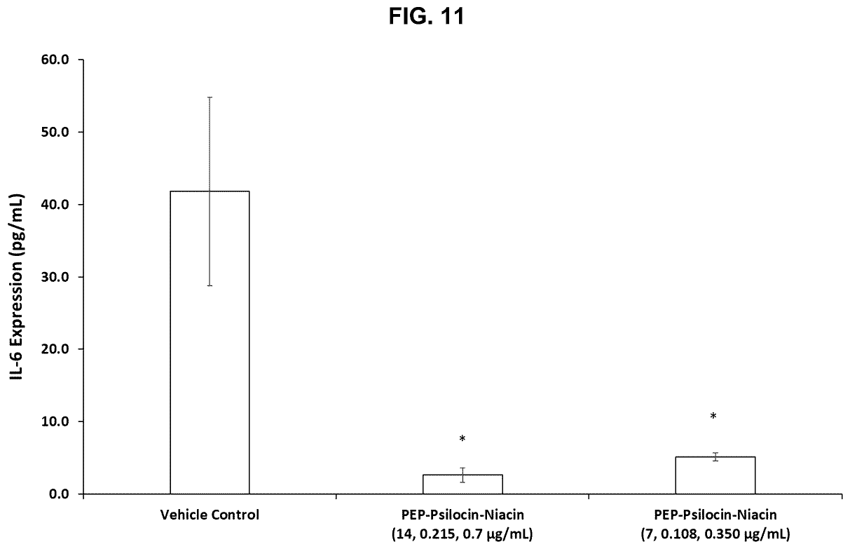

Human PBMCs were treated with 100 ng/mL LPS to induce inflammatory conditions. The PBMCs were then treated with different concentrations of He myc SOP EtOAc (He), niacin, PEP, psilocin, a He-psilocin-niacin formulation, a PEP-psilocin-niacin formulation, or DMSO vehicle control, and IL-6 expression was measured (pg/mL). The IL-6 expression results for each treatment are shown in FIG. 6-11. FIG. 10-11 specifically demonstrate that multiple concentrations of He-psilocin-niacin and PEP-psilocin-niacin formulations can significantly reduce the expression of IL-6, indicating an anti-inflammatory effect for the combination formulations.

### Example 9

### The impact of Norbaeocystin on IL-6 Expression in Human PBMCs

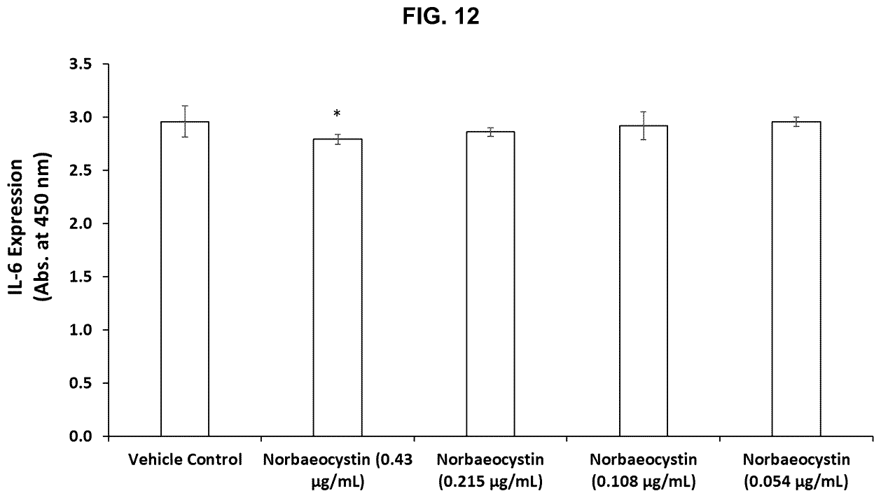

Human PBMCs were treated with 100 ng/mL LPS to induce inflammatory conditions. The PBMCs were then treated with different concentrations of Norbaeocystin or DMSO vehicle control, and IL-6 expression was measured (average adjusted absorbance at 450 nm). The IL-6 expression results for each treatment are shown in FIG. 12, where the highest concentration of Norbaeocystin (0.43 μg/mL) significantly reduced IL-6 expression, indicating an anti-inflammatory effect.

### Example 10

### The impact of Different Individual Components and Combination Formulations on TNF-α Expression in Human PBMCs

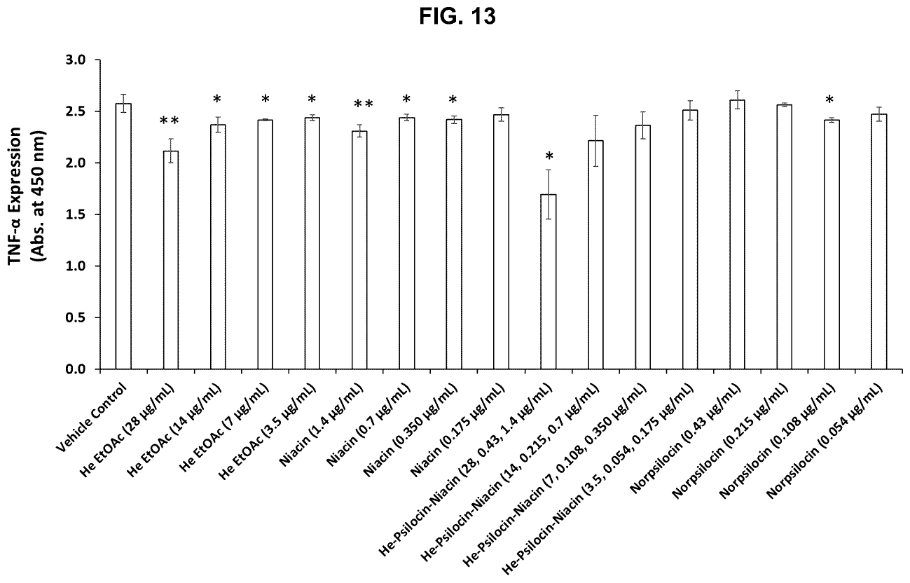

Human PBMCs were treated with 100 ng/mL LPS to induce inflammatory conditions. The PBMCs were then treated with different concentrations of He EtOAc (He), niacin, norpsilocin, a He-psilocin-niacin combination formulation, or DMSO vehicle control, and TNF-α expression was measured (average adjusted absorbance at 450 nm). The TNF-α expression results for each treatment are shown in FIG. 13, where the He-psilocin-niacin formulation (28, 0.43, 1.4 μg/mL) was found to significantly reduce TNF-α expression to levels lower than any of the tested individual components. These data indicate a potential additive or synergistic anti-inflammatory effect for the combination formulation, and also indicate that the individual components of He EtOAc (He), niacin, and norpsilocin have anti-inflammatory effects at certain concentrations.

What is claimed is:

1. A composition comprising:
one or more tryptamines, salts thereof, or combinations thereof; and
extracts or isolates from Hericium erinaceus mushroom species, erinacines, hericenones, or combinations thereof.

2. The composition of claim 1, wherein the one or more tryptamines are psilocybin, psilocin, norpsilocin, baeocystin, norbaeocystin, N,N-dimethyltryptamine (DMT), or combinations thereof.

3. The composition of claim 1, wherein the composition comprises about 1 ng to about 10 mg, about 10 mg to about 100 mg, about 10 mg to about 20 mg, about 20 mg to about 50 mg, about 20 mg to about 100 mg, about 1 ng to about 20 mg, about 1 ng to about 50 mg, or about 1 ng to about 100 mg of the one or more tryptamines, salts thereof, or combinations thereof.

4. The composition of claim 2, wherein the composition comprises about 1 ng to about 2000 mg of the extracts or isolates from Hericium erinaceus mushroom species, erinacines, hericenones, or combinations thereof.

5. The composition of claim 1, further comprising a monoamine oxidase inhibitor.

6. The composition of claim 5, wherein the composition comprises about 70 mg to about 200 mg of the monoamine oxidase inhibitor. 7 The composition of claim 5, wherein the monoamine oxidase inhibitor is Norharman, Harmine, 1,2,3,4-tetrahydro-β-carboline-3-carboxylic acid, 1-methyl-1,2,3,4-tetrahydro-β-carboline-3-carboxylic acid, 1-methyl-2,3,4,9-tetrahydro-1H-β-carboline-1,3-dicarboxylic acid, Harmaline, N-methoxy-1-vinyl-β-carboline, ethyl 9H-β-arboline-3-carboxylate, 1-furyl-β-carboline-3-carboxylic acid, 1-[5-(methoxymethyl)-2-furyl]-9H-β-carboline-3-carboxylic acid, 6-hydroxy-3-(6-hydroxy-1H-indol-3-yl)-9H-β-carboline-4-carboxylic acid, Strictosidine, (1S)-1-{[(2S,3R,4S)-2-(β-L-glucopyranosyloxy)-5-(methoxycarbonyl)-3-vinyl-3,4-dihydro-2H-pyran-4-yl]methyl}-2,3,4,9-tetrahydro-1H-β-carboline-1,3-dicarboxylic acid, or combinations thereof.

8. A composition comprising:
psilocybin, psilocin, norpsilocin, baeocystin, norbaeocystin, N,N-dimethyltryptamine (DMT), salts thereof, or combinations thereof; and
an erinacine or hericenone in pure form, extracts or isolates from Hericium erinaceus mushroom species, or combinations thereof.

9. The composition of claim 8, wherein the composition comprises about 1 ng to about 10 mg, about 10 mg to about 100 mg, about 10 mg to about 20 mg, about 20 mg to about 50 mg, about 20 mg to about 100 mg, about 1 ng to about 20 mg, about 1 ng to about 50 mg, or about 1 ng to about 100 mg of the psilocybin, psilocin, norpsilocin, baeocystin, norbaeocystin, N,N-dimethyltryptamine (DMT), salts thereof, or combinations thereof.

10. The composition of claim 8, wherein the composition comprises about 1 ng to about 2000 mg of the erinacine or hericenone in pure form, extracts or isolates from Hericium erinaceus mushroom species, or combinations thereof.

11. The composition of claim 8, further comprising a monoamine oxidase inhibitor.

12. The composition of claim 11, wherein the composition comprises about 70 mg to about 200 mg of the monoamine oxidase inhibitor.

13. The composition of claim 11, wherein the monoamine oxidase inhibitor is Norharman, Harmine, 1,2,3,4-tetrahydro-β-carboline-3-carboxylic acid, 1-methyl-1,2,3,4-tetrahydro-β-carboline-3-carboxylic acid, 1-methyl-2,3,4,9-tetrahydro-1H-β-carboline-1,3-dicarboxylic acid, Harmaline, N-methoxy-1-vinyl-β-carboline, ethyl 9H-β-arboline-3-carboxylate, 1-furyl-β-carboline-3-carboxylic acid, 1-[5-(methoxymethyl)-2-furyl]-9H-β-carboline-3-carboxylic acid, 6-hydroxy-3-(6-hydroxy-1H-indol-3-yl)-9H-β-carboline-4-carboxylic acid, Strictosidine, (1S)-1-{[(2S,3R,4S)-2-(β-L-glucopyranosyloxy)-5-(methoxycarbonyl)-3-vinyl-3,4-dihydro-2H-pyran-4-yl]methyl}-2,3,4,9-tetrahydro-1H-β-carboline-1,3-dicarboxylic acid, or combinations thereof.

14. A method for treating or modulating an inflammatory response triggered by an infectious disease or condition, the method comprising:
administering a composition to a subject in need thereof, the composition comprising:
one or more tryptamines, salts thereof, or combinations thereof.

15. A method for treating or modulating an inflammatory response triggered by an infectious disease or condition, the method comprising:
administering a composition to a subject in need thereof, the composition comprising:
one or more tryptamines, salts thereof, or combinations thereof; and
extracts or isolates from Hericium erinaceus mushroom species, erinacines, hericenones, or combinations thereof.

16. The method of claim 14, wherein the composition comprises about 1 ng to about 10 mg, about 10 mg to about 100 mg, about 10 mg to about 20 mg, about 20 mg to about 50 mg, about 20 mg to about 100 mg, about 1 ng to about 20 mg, about 1 ng to about 50 mg, or about 1 ng to about 100 mg of the one or more tryptamines, salts thereof, or combinations thereof.

17. The method of claim 14, wherein the one or more tryptamines are psilocybin, psilocin, norpsilocin, baeocystin, norbaeocystin, N,N-dimethyltryptamine (DMT), or combinations thereof.

18. The method of claim 15, wherein the composition comprises about 1 ng to about 2000 mg of the extracts or isolates from Hericium erinaceus mushroom species, erinacines, hericenones, or combinations thereof.

19. The method of claim 14, wherein the composition further comprises a monoamine oxidase inhibitor.

20. The method of claim 19, wherein the composition comprises about 70 mg to about 200 mg of the monoamine oxidase inhibitor.

21. The method of claim 19, wherein the monoamine oxidase inhibitor is Norharman, Harmine, 1,2,3,4-tetrahydro-β-carboline-3-carboxylic acid, 1-methyl-1,2,3,4-tetrahydro-β-carboline-3-carboxylic acid, 1-methyl-2,3,4,9-tetrahydro-1H-β-carboline-1,3-dicarboxylic acid, Harmaline, N-methoxy-1-vinyl-β-carboline, ethyl 9H-β-arboline-3-carboxylate, 1-furyl-β-carboline-3-carboxylic acid, 1-[5-(methoxymethyl)-2-furyl]-9H-β-carboline-3-carboxylic acid, 6-hydroxy-3-(6-hydroxy-1H-indol-3-yl)-9H-β-carboline-4-carboxylic acid, Strictosidine, (1S)-1-{[(2S,3R,4S)-2-(β-L-glucopyranosyloxy)-5-(methoxycarbonyI)-3-vinyl-3,4-dihydro-2H-pyran-4-yl]methyl}-2,3,4,9-tetrahydro-1H-β-carboline-1,3-dicarboxylic acid, or combinations thereof.

22. The method of claim 14, wherein the inflammatory response is cytokine storm.

23. The method of claim 14, wherein the infectious disease or condition is a viral infection, a bacterial infection, or a parasitic infection.

24. The method of claim 23, wherein the viral infection is Paramyxoviridae (respiratory syncytial virus (RSV), parainfluenza virus (PIV), metapneumovirus (MPV), enteroviruses), Picornaviridae (Rhinovirus, RV), Coronaviridae (CoV), Adenoviridae (Adenovirus), Parvoviridae (HBoV), Orthomyxoviridae (influenza A, B, C, D, Isavirus, Thogotovirus, Quaranjavirus), Herpesviridae (human herpes viruses, Varicella zoster virus, Epstein-Barr virus, cytomegalovirus), avian influenza, smallpox, pandemic influenza, or adult respiratory distress syndrome (ARDS).

25. The method of claim 23, wherein the bacterial infection is Streptococcus pneumoniae, Mycobacterium tuberculosis, Bordetella pertussis, Haemophilus influenzae, Moraxella catarrhalis, Pseudomonas aeruginosa, Stenotrophomonas maltophila, Staphylococcus aureus, Streptococcus pyogenes, Neisseria meningitidis, Klebsiella pneumoniae, or Non-tuberculosis Mycobacterium.

26. The method of claim 23, wherein the parasitic infection is malaria.

27. The method of claim 14, wherein inflammation is reduced and neuroregeneration is induced in the subject.

28. The method of claim 27, wherein neuroregeneration comprises neurite outgrowth.

29. A method for inducing expression of an anti-inflammatory cytokine, the method comprising administering a composition to a subject in need thereof, the composition comprising: one or more tryptamines, salts thereof, or combinations thereof.

30. A method for inducing expression of an anti-inflammatory cytokine, the method comprising administering a composition to a subject in need thereof, the composition comprising:
one or more tryptamines, salts thereof, or combinations thereof; and
extracts or isolates from Hericium erinaceus mushroom species, erinacines, hericenones, or combinations thereof.

31. The method of claim 29, wherein the composition comprises about 1 ng to about 10 mg, about 10 mg to about 100 mg, about 10 mg to about 20 mg, about 20 mg to about 50 mg, about 20 mg to about 100 mg, about 1 ng to about 20 mg, about 1 ng to about 50 mg, or about 1 ng to about 100 mg of the one or more tryptamines, salts thereof, or combinations thereof.

32. The method of claim 29, wherein the one or more tryptamines are psilocybin, psilocin, norpsilocin, baeocystin, norbaeocystin, N,N-dimethyltryptamine (DMT), or combinations thereof.

33. The method of claim 30, wherein the composition comprises about 1 ng to about 2000 mg of the extracts or isolates from Hericium erinaceus mushroom species, erinacines, hericenones, or combinations thereof.

34. The method of claim 29, wherein the composition further comprises a monoamine oxidase inhibitor.

35. The method of claim 34, wherein the composition comprises about 70 mg to about 200 mg of the monoamine oxidase inhibitor.

36. The method of claim 34, wherein the monoamine oxidase inhibitor is Norharman, Harmine, 1,2,3,4-tetrahydro-β-carboline-3-carboxylic acid, 1-methyl-1,2,3,4-tetrahydro-β-carboline-3-carboxylic acid, 1-methyl-2,3,4,9-tetrahydro-1H-β-carboline-1,3-dicarboxylic acid, Harmaline, N-methoxy-1-vinyl-β-carboline, ethyl 9H-β-arboline-3-carboxylate, 1-fury)-β-carboline-3-carboxylic acid, 1-[5-(methoxymethyl)-2-furyl]-9H-β-carboline-3-carboxylic acid, 6-hydroxy-3-(6-hydroxy-1H-indol-3-yl)-9H-β-carboline-4-carboxylic acid, Strictosidine, (1S)-1-{[(2S,3R,4S)-2-(β-L-glucopyranosyloxy)-5-(methoxycarbonyI)-3-vinyl-3,4-dihydro-2H-pyran-4-yl]methyl}-2,3,4,9-tetrahydro-1H-β-carboline-1,3-dicarboxylic acid, or combinations thereof.

37. The method of claim 29, wherein the anti-inflammatory cytokine is IL-4, IL-10, IL-1RA, or a combination thereof.

38. The method of claim 29, wherein inflammation is reduced and neuroregeneration is induced in the subject.

39. The method of claim 38, wherein neuroregeneration comprises neurite outgrowth.

40. A method for treating or modulating an inflammatory response triggered by an infectious disease or condition by inducing expression of one or more anti-inflammatory cytokines selected from the group of IL-4, IL-10, and IL-1RA, the method comprising:
administering a composition to a subject in need thereof, the composition comprising:
about 1 ng to about 10 mg, about 10 mg to about 100 mg, about 10 mg to about 20 mg, about 20 mg to about 50 mg, about 20 mg to about 100 mg, about 1 ng to about 20 mg, about 1 ng to about 50 mg, or about 1 ng to about 100 mg of one or more tryptamines, salts thereof, or combinations thereof; and
about 10 ng to about 2000 mg of extracts or isolates from Hericium erinaceus mushroom species, erinacines, hericenones, or combinations thereof.

41. A method for treating or modulating an inflammatory response triggered by an infectious disease or condition by inducing expression of one or more anti-inflammatory cytokines selected from the group of IL-4, IL-10, and IL-1RA, the method comprising:
administering a composition to a subject in need thereof, the composition comprising:
about 1 ng to about 10 mg, about 10 mg to about 100 mg, about 10 mg to about 20 mg, about 20 mg to about 50 mg, about 20 mg to about 100 mg, about 1 ng to about 20 mg, about 1 ng to about 50 mg, or about 1 ng to about 100 mg of one or more tryptamines, salts thereof, or combinations thereof;
about 1 ng to about 2000 mg of extracts or isolates from Hericium erinaceus mushroom species, erinacines, hericenones, or combinations thereof; and
about 70 mg to about 200 mg of a monoamine oxidase inhibitor.

42. The method of claim 40, wherein the inflammatory response is cytokine storm.

43. The method of claim 40, wherein the infectious disease or condition is a viral infection, a bacterial infection, or a parasitic infection.

44. The method of claim 43, wherein the viral infection is Paramyxoviridae (respiratory syncytial virus (RSV), parainfluenza virus (PIV), metapneumovirus (MPV), enteroviruses), Picornaviridae (Rhinovirus, RV), Coronaviridae (CoV), Adenoviridae (Adenovirus), Parvoviridae (HBoV), Orthomyxoviridae (influenza A, B, C, D, Isavirus, Thogotovirus, Quaranjavirus), Herpesviridae (human herpes viruses, Varicella zoster virus, Epstein-Barr virus, cytomegalovirus), avian influenza, smallpox, pandemic influenza, or adult respiratory distress syndrome (ARDS).

45. The method of claim 43, wherein the bacterial infection is Streptococcus pneumoniae, Mycobacterium tuberculosis, Bordetella pertussis, Haemophilus influenzae, Moraxella catarrhalis, Pseudomonas aeruginosa, Stenotrophomonas maltophila, Staphylococcus aureus, Streptococcus pyogenes, Neisseria meningitidis, Klebsiella pneumoniae, or Non-tuberculosis Mycobacterium.

46. The method of claim 43, wherein the parasitic infection is malaria.

47. The method of claim 40, wherein the one or more tryptamines are psilocybin, psilocin, norpsilocin, baeocystin, norbaeocystin, N,N-dimethyltryptamine (DMT), or combinations thereof.

48. The method of claim 40, wherein the monoamine oxidase inhibitor is Norharman, Harmine, 1,2,3,4-tetrahydro-β-carboline-3-carboxylic acid, 1-methyl-1,2,3,4-tetrahydro-β-carboline-3-carboxylic acid, 1-methyl-2,3,4,9-tetrahydro-1H-β-carboline-1,3-dicarboxylic acid, Harmaline, N-methoxy-1-vinyl-β-carboline, ethyl 9H-β-arboline-3-carboxylate, 1-furyl-β-carboline-3-carboxylic acid, 1-[5-(methoxymethyl)-2-furyl]-9H-β-carboline-3-carboxylic acid, 6-hydroxy-3-(6-hydroxy-1H-indol-3-yl)-9H-β-carboline-4-carboxylic acid, Strictosidine, (1S)-1-{[(2S,3R,4S)-2-(β-L-glucopyranosyloxy)-5-(methoxycarbonyl)-3-vinyl-3,4-dihydro-2H-pyran-4-yl]methyl}-2,3,4,9-tetrahydro-1H-β-carboline-1,3-dicarboxylic acid, or combinations thereof.

49. The method of claim 40, wherein inflammation is reduced and neuroregeneration is induced in the subject.

50. The method of claim 49, wherein neuroregeneration comprises neurite outgrowth.

51. The method of claim 14, wherein the infectious disease or condition causes neurological damage in the subject and the method results in treatment of the neurological damage.

52. The method of claim 40, wherein the infectious disease or condition causes neurological damage in the subject and the method results in treatment of the neurological damage.
